(19) United States
(12) Patent Application Publication
(10) Pub. No.: US 2018/0238886 A1
(43) Pub. Date: Aug. 23, 2018

(54) METHODS FOR TREATING HEMATOLOGICAL CANCER AND THE USE OF BIOMARKERS AS A PREDICTOR FOR RESPONSIVENESS TO TREATMENT COMPOUNDS

(71) Applicant: CELGENE CORPORATION, Summit, NJ (US)

(72) Inventors: Anita GANDHI, Bernardsville, NJ (US); Matthew William Burnell TROTTER, Sevilla (ES); Alberto RISUEÑO-PEREZ, Sevilla (ES); Fadi George TOWFIC, Somerville, MA (US)

(21) Appl. No.: 15/884,221

(22) Filed: Jan. 30, 2018

Related U.S. Application Data
(60) Provisional application No. 62/452,863, filed on Jan. 31, 2017.

(51) Int. Cl. G01N 33/574; C12Q 1/6886; G01N 33/50

(52) U.S. Cl. CPC G01N 33/57407; G01N 33/57426; C12Q 1/6886; G01N 2800/52; C12Q 2600/156; C12Q 2600/158; G01N 33/5094

(57) ABSTRACT

A method for predicting the responsiveness of a patient having a hematological cancer to a treatment compound, comprising obtaining a biological sample from the patient having the hematological cancer; determining the expression level of one, two, three, four, five, or more genes; and comparing the expression level of the one, two, three, four, five, or more genes in the biological sample with a reference expression level of the same genes, wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

### CROSS REFERENCE

This application claims the benefit of priority of U.S. Provisional Patent Application No. 62/452,863, filed Jan. 31, 2017, the content of which is incorporated herein by reference in its entirety.

### 1. FIELD

The present application relates generally to methods for predicting whether a patient having a hematological cancer will be responsive to a treatment compound such as lenalidomide or 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or polymorph thereof. More specifically, the present application relates to utilizing biomarkers or classifiers that correlate with responsiveness to lenalidomide or Compound A.

Also provided herein are said treatment compounds for use in methods of treating a patient having a hematological cancer wherein the treatment comprises predicting whether the patient will be responsive to said treatment compound by utilizing biomarkers or classifiers that correlate with responsiveness to lenalidomide or Compound A.

### 2. BACKGROUND

2.1 Hematological Cancer

Hematological cancer generally includes three main types: lymphoma, leukemia, and myeloma. Lymphoma refers to cancers that originate in the lymphatic system. Lymphoma includes, but is not limited to, Hodgkin's lymphoma, non-Hodgkin's lymphoma (NHL), diffuse large B-cell lymphoma (DLBCL), and peripheral T-cell lymphomas (PTCL), etc. Leukemia refers to malignant neoplasms of the blood-forming tissues. Acute leukemia involves predominantly undifferentiated cell populations, whereas chronic leukemia involves more mature cell forms. Acute leukemia is divided into acute lymphoblastic leukemia (ALL) and acute myeloblastic leukemia (AML) types. The Merck Manual, 946-949 (17th ed. 1999). Chronic leukemia is divided into chronic lymphocytic leukemia (CLL) or chronic myelocytic leukemia (CML). The Merck Manual, 949-952 (17th ed. 1999). Myeloma is a cancer of plasma cells in the bone marrow. Because myeloma frequently occurs at many sites in the bone marrow, it is often referred to as multiple myeloma (MM).

2.2 Pathobiology of DLBCL

The non-Hodgkin lymphomas (NHLs) are a diverse group of blood cancers that include any kind of lymphoma except Hodgkin's lymphomas. Types of NHL vary significantly in their severity, from indolent to very aggressive. Less aggressive non-Hodgkin lymphomas are compatible with a long survival while more aggressive non-Hodgkin lymphomas can be rapidly fatal without treatment. They can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Prognosis and treatment depend on the stage and type of disease.

DLBCL accounts for approximately one-third of non-Hodgkin's lymphomas. While some DLBCL patients are cured with traditional chemotherapy, the remainder dies from the disease. Anticancer drugs cause rapid and persistent depletion of lymphocytes, possibly by direct apoptosis induction in mature T and B cells. See K. Stahnke. et al., Blood 2001, 98:3066-3073. Absolute lymphocyte count (ALC) has been shown to be a prognostic factor in follicular non-Hodgkin's lymphoma and recent results have suggested that ALC at diagnosis is an important prognostic factor in diffuse large B-cell lymphoma.

DLBCL can be divided into distinct molecular subtypes according to their gene profiling patterns: germinal-center B-cell-like DLBCL (GCB-DLBCL), activated B-cell-like DLBCL (ABC-DLBCL), primary mediastinal B-cell lymphoma (PMBL), or not otherwise specified DLBCL (NOS-DLBCL). These subtypes are characterized by distinct differences in survival, chemo-responsiveness, and signaling pathway dependence, particularly the NF-□B pathway. See D. Kim et al., Journal of Clinical Oncology, 2007 ASCO Annual Meeting Proceedings Part I. Vol 25, No. 18S (June 20 Supplement), 2007: 8082. See Bea S, et al., Blood 2005; 106: 3183-90; Ngo V. N. et al., Nature 2011; 470: 115-9. Such differences have prompted the search for more effective and subtype-specific treatment strategies in DLBCL.

The incidence of cancer continues to climb as the general population ages, as new cancers develop, and as susceptible populations grow. A tremendous demand therefore exists for new methods and compositions that can be used to treat patients with NHL including DLBCL.

2.3 Methods of Treatment

Current cancer therapy, in general, can involve surgery, chemotherapy, hormonal therapy and/or radiation treatment to eradicate neoplastic cells in a patient (see, for example, Stockdale, 1998, Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Biological therapies and immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A majority of cancer chemotherapeutics act by inhibiting DNA synthesis, either directly or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove refractory to standard chemotherapeutic treatment protocols.

In the context of DLBCL, treatment usually includes administration of a combination of chemotherapy and antibody therapy. The most widely used treatment of DLBCL is a mixture of the antibody rituximab and several chemotherapy drugs (cyclophosphamide, doxorubicin, vincristine, and prednisone (R-CHOP), and in some cases etoposide is added (R-EPOCH)). DLBCL also typically requires immediate treatment upon diagnosis due to how quickly the disease can advance. For some patients, DLBCL returns or becomes refractory following treatment. Several alternative treatments, some of which can include use of lenalidomide, are currently being tested in clinical trials for patients with newly diagnosed, relapsed or refractory DLBCL.

Thus, there is a significant need for safe and effective methods of treating, preventing and managing cancers including DLBCL, particularly for cancers that are refractory to standard treatments, such as surgery, radiation therapy, chemotherapy and hormonal therapy, while reducing or avoiding the toxicities and/or side effects associated with the conventional therapies. Moreover, there remains a need for the ability to predict and monitor response to therapy in order to increase the quality of care for patients, avoid unnecessary treatment and to increase the success rate in treating cancer, including DLBCL, in clinical practice.

### 3. SUMMARY

The present disclosure is based, in part, on the discovery that pre-treatment transcriptional profiles derived from tumor biopsies obtained from a patient having a hematological cancer (e.g., DLBCL) can be used to predict responsiveness of the patient having the hematological cancer (e.g., DLBCL) to treatment with lenalidomide and/or Compound A.

In one aspect, provided herein are methods for predicting the responsiveness of a patient having a hematological cancer to a treatment compound.

In some embodiments, provided herein is a method for predicting the responsiveness of a patient having a hematological cancer to a treatment compound, comprising:

(a) obtaining a biological sample from the patient having the hematological cancer;

(b) determining the expression level of one, two, three, four, five, or more of the genes identified in Tables 1, 2, 3, and/or 4; and

(c) comparing the expression level of the one, two, three, four, five, or more of the genes identified in Tables 1, 2, 3, and/or 4 in the biological sample with a reference expression level of the same genes;

wherein the differential expression level of the one, two, three, four, five, or more of the genes in the biological sample relative to the reference expression level of the one, two, three, four, five, or more of the genes indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In certain embodiments, the reference expression level of the one, two, three, four, five, or more of the genes identified in Tables 1, 2, 3, and/or 4 is the expression level of the same genes in a reference sample. In one embodiment, the reference sample is a second biological sample. In another embodiment, the reference sample is synthetically derived.

In certain embodiments, provided herein is a method for predicting the responsiveness of a patient having a hematological cancer to a treatment compound, comprising:

(a) obtaining a first biological sample from a first patient having the hematological cancer;

(b) determining the expression level of one, two, three, four, five, or more of the genes identified in Tables 1, 2, 3, and/or 4; and

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is not responsive to the treatment compound,

wherein the differential expression level of the one, two, three, four, five, or more of the genes in the first biological sample relative to the expression level of the one, two, three, four, five, or more of the genes in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, provided herein is a method for predicting the responsiveness of a patient having a hematological cancer to a treatment compound, comprising:

(a) obtaining a first biological sample from a first patient having the hematological cancer;

(b) determining the expression level of one, two, three, four, five, or more of the genes identified in Tables 1, 2, 3, and/or 4; and

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is responsive to the drug,

wherein the similar expression level of the one, two, three, four, five, or more of the genes in the first biological sample relative to the expression level of the one, two, three, four, five, or more of the genes in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In other embodiments, provided herein is a method for predicting the responsiveness of a patient having a hematological cancer to a treatment compound, comprising:

(a) obtaining a first biological sample from a first patient having the hematological cancer;

(b) determining the expression level of one, two, three, four, five, or more of the genes identified in Tables 1, 2, 3, and/or 4; and

(c) comparing the gene expression profile of the genes or subset of genes in the first biological sample to
(i) the gene expression profile of the one, two, three, four, five, or more of the genes in a second biological sample from a second patient having the hematological cancer, wherein the second patient is responsive to the treatment compound, and
(ii) the gene expression profile of the one, two, three, four, five, or more of the genes in a third biological sample from a third patient having the hematological cancer, wherein the third patient is not responsive to the treatment compound,

wherein the similar expression level of the one, two, three, four, five, or more of the genes in the first biological sample relative to the expression level of the one, two, three, four, five, or more of the genes in the second biological sample indicates that the first patient will be responsive to the treatment compound, and the similar expression level of the one, two, three, four, five, or more of the genes in the first biological sample relative to the expression level of the one, two, three, four, five, or more of the genes in the third biological sample indicates that the first patient will not be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, the one, two, three, four, five, or more of genes are a subset of the genes in Table 1, a subset of the genes in Table 2, a subset of the genes in Table 3, and/or a subset of the genes in Table 4. In other embodiments, the one, two, three, four, five, or more of genes are a combination of a subset of the genes in two tables selected from Tables 1-4. In yet other embodiments, the one, two, three, four, five, or more of genes are a combination of a subset of the genes in three tables selected from Tables 1-4. In still other embodiments, the one, two, three, four, five, or more of genes are a combination of a subset of the genes in Table 1, a subset of the genes in Table 2, a subset of the genes in Table 3, and a subset of the genes in Table 4.

In another aspect, provided herein are methods for predicting the responsiveness of a patient having a hematological cancer to a treatment compound, comprising: (a) obtaining a biological sample from a patient having the hematological cancer; (b) determining the expression of the genes or a certain subset of genes set forth in Tables 1, 2, 3 and/or 4, or any combination thereof, in the biological sample and generating a score based on the expression of the genes or subset of genes, (c) comparing the score to a reference score, wherein a score similar to a reference score indicates that the patient will be responsive to the treatment compound. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In another aspect, provided herein are methods for predicting the responsiveness of a patient having a hematological cancer to a treatment compound, comprising: (a) obtaining a biological sample from a patient having the hematological cancer; (b) determining the expression of the genes or a certain subset of genes set forth in Tables 1, 2, 3, and/or 4, or any combination thereof, in the biological sample and generating a score based on the expression of the genes or subset of genes, (c) comparing the score to a first reference score and a second reference score, wherein (i) the score similar to the first reference score indicates that the patient will be responsive to the treatment compound, and/or (ii) the score similar to the second reference score indicates that the patient will be responsive to the treatment compound. In specific embodiments, the first reference score is based on the expression of the same genes or subset of genes in a population of patients having the hematological cancer who are responsive to the treatment compound, and the second reference score is based on the expression of the same genes or subset of genes in a population of patients having the hematological cancer who are not responsive to the treatment compound. In a specific embodiment, the treatment compound is a compound disclosed herein. In one embodiment, the treatment compound is lenalidomide. In another embodiment, the treatment compound is Compound A.

In another aspect, provided herein are methods for treating a patient having a hematological cancer with a treatment compound. Accordingly, provided herein is a treatment compound for use in said methods for treating a patient having a hematological cancer.

In some embodiments, provided herein is a method for treating a patient having a hematological cancer with a treatment compound, comprising:

(a) obtaining a biological sample from the patient having the hematological cancer;

(b) determining the expression level of one, two, three, four, five, or more of the genes identified in Tables 1, 2, 3, and/or 4;

(c) comparing the expression level of the one, two, three, four, five, or more of the genes identified in Tables 1, 2, 3, and/or 4 in the biological sample with a reference expression level of the same genes; and

(d) administering the treatment compound to the patient if the one, two, three, four, five, or more of the genes in the biological sample are differentially expressed relative to the reference expression level of the same genes;

wherein the differential expression level of the one, two, three, four, five, or more of the genes in the biological sample relative to the reference expression level of the one, two, three, four, five, or more of the genes indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. Accordingly, provided herein is said treatment compound for use in said method for treating a patient having a hematological cancer.

In certain embodiments, the reference expression level of the one, two, three, four, five, or more of the genes identified in Tables 1, 2, 3, and/or 4 is the expression level of the same genes in a reference sample. In one embodiment, the reference sample is a second biological sample. In another embodiment, the reference sample is synthetically derived.

In certain embodiments, provided herein is a method for treating a patient having a hematological cancer with a treatment compound, comprising:

(a) obtaining a first biological sample from a first patient having the hematological cancer;

(b) determining the expression level of one, two, three, four, five, or more of the genes identified in Tables 1, 2, 3, and/or 4;

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is not responsive to the treatment compound; and

(d) administering the treatment compound to the first patient if the one, two, three, four, five, or more of the genes in the first biological sample are differentially expressed relative to the expression level of the one, two, three, four, five, or more of the genes in the second biological sample,

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. Accordingly, provided herein is said treatment compound for use in said method for treating a patient having a hematological cancer.

In some embodiments, provided herein is a method for treating a patient having a hematological cancer with a treatment compound, comprising:

(a) obtaining a first biological sample from a first patient having the hematological cancer;

(b) determining the expression level of one, two, three, four, five, or more of the genes identified in Tables 1, 2, 3, and/or 4;

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is responsive to the treatment compound; and

(d) administering the treatment compound to the first patient if the one, two, three, four, five, or more of the genes in the first biological sample are similarly expressed relative to the expression level of the one, two, three, four, five, or more of the genes in the second biological sample,

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. Accordingly, provided herein is said treatment compound for use in said method for treating a patient having a hematological cancer.

In other embodiments, provided herein is a method for treating a patient having a hematological cancer with a treatment compound, comprising:

(a) obtaining a first biological sample from a first patient having the hematological cancer;

(b) determining the expression level of one, two, three, four, five, or more of the genes identified in Tables 1, 2, 3, and/or 4;

(c) comparing the gene expression profile of the genes or subset of genes in the first biological sample to
(i) the gene expression profile of the one, two, three, four, five, or more of the genes in a second biological sample from a second patient having the hematological cancer, wherein the second patient is responsive to the treatment compound, and
(ii) the gene expression profile of the one, two, three, four, five, or more of the genes in a third biological sample from a third patient having the hematological cancer, wherein the third patient is not responsive to the treatment compound; and

(d) administering the treatment compound to the first patient if: (i) the one, two, three, four, five, or more of the genes in the first biological sample are similarly expressed relative to the expression level of the one, two, three, four, five, or more of the genes in the second biological sample, and/or (ii) the one, two, three, four, five, or more of the genes in the first biological sample are differentially expressed relative to the expression level of the one, two, three, four, five, or more of the genes in the third biological sample,

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. Accordingly, provided herein is said treatment compound for use in said method for treating a patient having a hematological cancer.

In some embodiments, the one, two, three, four, five, or more of genes are a subset of the genes in Table 1, a subset of the genes in Table 2, a subset of the genes in Table 3, and/or a subset of the genes in Table 4. In other embodiments, the one, two, three, four, five, or more of genes are a combination of a subset of the genes in two tables selected from Tables 1-4. In yet other embodiments, the one, two, three, four, five, or more of genes are a combination of a subset of the genes in three tables selected from Tables 1-4. In still other embodiments, the one, two, three, four, five, or more of genes are a combination of a subset of the genes in Table 1, a subset of the genes in Table 2, a subset of the genes in Table 3, and a subset of the genes in Table 4.

In some embodiments of the various methods provided herein, expression levels of multiple genes (biomarkers) provided herein are used to predict a patient's response to the compound provided herein. When expression levels of multiple genes (biomarkers) are used to predict a patient's response to a treatment with the compound provided herein, e.g., lenalidomide or Compound A, any classifier that classifies based on two or more features can be used in the present disclosure.

In some embodiments, the methods provided herein further comprise (a) generating a score of the sample based on the expression levels of the genes or a subset thereof provided herein in the sample; and (b) determining the probability of the subject being responsive to the compound provided herein by comparing the score of the sample to a reference score.

In some more specific embodiments, the methods provided herein further comprise: (a) determining the expression levels of the genes or a subset thereof provided herein in a biological sample from a population of subjects that previously have been administered with the compound provided herein, (b) generating a score for the expression levels of the genes or a subset thereof provided herein for each subject of the population; (c) differentiating the subjects that are responsive to the compound provided herein from those subjects that are not responsive to the compound provided herein; and (d) determining responsiveness to the compound provided herein based on the scores for the subjects that are responsive to the compound and those subjects that are not responsive to the compound.

In other more specific embodiments, the methods provided herein further comprise: (a) determining the expression levels of the genes or a subset thereof provided herein in a biological sample from a population of subjects that previously have been administered with the compound, (b) generating a score for the expression levels of the genes or a subset thereof provided herein for each subject of the population; (c) differentiating the subjects that are responsive to the compound provided herein from those subjects that are not responsive to compound provided herein; and (d) generating a reference score that is predictive of the responsiveness of a subject to the compound provided herein using a model based on the scores for the subjects that are responsive to the compound and those subjects that are not responsive to the compound.

In another aspect, provided herein are methods for treating a patient having a hematological cancer with a treatment compound, comprising: (a) obtaining a biological sample from a patient having the hematological cancer; (b) determining the expression of the genes or a certain subset of genes set forth in Tables 1, 2, 3, and/or 4, or any combination thereof, in the biological sample and generating a score based on the expression of the genes or subset of genes, (c) comparing the score to a reference score; and (d) administering the treatment compound to the patient if the score is similar to the reference score. Accordingly, provided herein is said treatment compound for use in said method for treating a patient having a hematological cancer. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In another aspect, provided herein are methods for treating a patient having a hematological cancer with a treatment compound, comprising: (a) obtaining a biological sample from a patient having the hematological cancer; (b) determining the expression of the genes or a certain subset of genes set forth in Tables 1, 2, 3, and/or 4, or any combination thereof, in the biological sample and generating a score based on the expression of the genes or subset of genes, (c) comparing the score to a first reference score and a second reference score; and (d) administering the treatment compound to the patient if (i) the score is similar to the first reference score, and/or (ii) the score is different from the second reference score. Accordingly, provided herein is said treatment compound for use in said method for treating a patient having a hematological cancer.

In specific embodiments, the first reference score is based on the expression of the same genes or subset of genes in a population of patients having the hematological cancer who are responsive to the treatment compound, and the second reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In certain embodiments, the hematological cancer is selected from the group consisting of leukemia, lymphoma, and multiple myeloma. In one embodiment, the hematological cancer is leukemia. In another embodiment, the hematological cancer is lymphoma. In still another embodiment, the hematological cancer is multiple myeloma.

In some embodiments, the lymphoma is DLBCL. In one embodiment, the DLBCL is refractory in the first patient. In another embodiment, the DLBCL is relapsed in the first patient. In yet another embodiment, the DLBCL is a germinal center B-cell-like subtype (GCB-DLBCL) in the first patient. In still another embodiment, the DLBCL is activated B-cell like subtype (ABC-DLBCL) in the first patient. In yet another embodiment, the DLBCL is not otherwise specified (NOS-DLBCL) in the first patient.

In some embodiments of the various methods provided herein, the expression of all of the genes in Table 1 is determined and compared. In some embodiments of the various methods provided herein, the expression of all of the genes in Table 2 is determined and compared. In some embodiments of the various methods provided herein, the expression of all of the genes in Table 3 is determined and compared. In some embodiments of the various methods provided herein, the expression of all of the genes in Table 4 is determined and compared.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of B cells in the first tumor sample, and (c) comparing the proportion of B cells in the first tumor sample with the proportion of B cells in a second tumor sample from a second patient having the same type of hematological cancer, wherein the second patient's hematological cancer is clinically insensitive to treatment with an immunomodulatory therapy, and wherein a lower proportion of B cells in the first tumor sample is measured relative the proportion of B cells in the second tumor sample indicates that the hematological cancer in the first patient will be clinically sensitive to treatment with the immunomodulatory therapy. In certain embodiments, the B cells are CD20+ B cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of dendritic cells in the first tumor sample, and (c) comparing the proportion of dendritic cells in the first tumor sample with the proportion of dendritic cells in a second tumor sample from a second patient having the same type of hematological cancer, wherein the second patient's hematological cancer is clinically insensitive to treatment with an immunomodulatory therapy, and wherein a higher proportion of dendritic cells in the first tumor sample is measured relative the proportion of dendritic cells in the second tumor sample indicates that the hematological cancer in the first patient will be clinically sensitive to treatment with the immunomodulatory therapy. In certain embodiments, the dendritic cells are CD11c+ dendritic cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of T cells in the first tumor sample, and (c) comparing the proportion of T cells in the first tumor sample with the proportion of T cells in a second tumor sample from a second patient having the same type of hematological cancer, wherein the second patient's hematological cancer is clinically insensitive to treatment with an immunomodulatory therapy, and wherein a higher proportion of T cells in the first tumor sample is measured relative the proportion of T cells in the second tumor sample indicates that the hematological cancer in the first patient will be clinically sensitive to treatment with the immunomodulatory therapy. In certain embodiments, the T cells are CD3+ T cells. In certain embodiments, the T cells are CD8+ T cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of macrophage cells in the first tumor sample, and (c) comparing the proportion of macrophage cells in the first tumor sample with the proportion of macrophage cells in a second tumor sample from a second patient having the same type of hematological cancer, wherein the second patient's hematological cancer is clinically insensitive to treatment with an immunomodulatory therapy, and wherein a higher proportion of macrophage cells in the first tumor sample is measured relative the proportion of macrophage cells in the second tumor sample indicates that the hematological cancer in the first patient will be clinically sensitive to treatment with the immunomodulatory therapy. In certain embodiments, the macrophage cells are CD163+ macrophage cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of PD-L1+ cells in the first tumor sample, and (c) comparing the proportion of PD-L1+ cells in the first tumor sample with the proportion of PD-L1+ cells in a second tumor sample from a second patient having the same type of hematological cancer, wherein the second patient's hematological cancer is clinically insensitive to treatment with an immunomodulatory therapy, and wherein a lower proportion of PD-L1+ cells in the first tumor sample is measured relative the proportion of PD-L1+ cells in the second tumor sample indicates that the hematological cancer in the first patient will be clinically sensitive to treatment with the immunomodulatory therapy. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are for predicting the clinical sensitivity of a hematological cancer to treatment with an immunomodulatory therapy comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and wherein that the proportion of the immune cells in the first tumor sample is (i) similar to the proportion of the same immune cells in tumor samples from patients having the same type of hematological cancer which are clinically sensitive to the immunomodulatory therapy, and (ii) not similar to the proportion of the same immune cells in tumor samples from patients having the same type of hematological cancer which are clinically insensitive to the immunomodulatory therapy indicates that the hematological cancer in the first page will be clinically sensitive to treatment with the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for managing or treating a hematological cancer comprising: (a) obtaining a first tumor sample from a first patient having the hematological cancer, (b) measuring the proportion of B cells in the first tumor sample, (c) comparing the proportion of B cells in the first tumor sample with the proportion of B cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a lower proportion of B cells in the first tumor sample is measured relative the proportion of B cells in the second tumor sample. In certain embodiments, the B cells are CD20+ B cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for managing or treating a hematological cancer comprising: (a) obtaining a first tumor sample from a first patient having the hematological cancer, (b) measuring the proportion of dendritic cells in the first tumor sample, (c) comparing the proportion of dendritic cells in the first tumor sample with the proportion of dendritic cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of dendritic cells in the first tumor sample is measured relative the proportion of dendritic cells in the second tumor sample. In certain embodiments, the dendritic cells are CD11c+ dendritic cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for managing or treating a hematological cancer comprising: (a) obtaining a first tumor sample from a first patient having the hematological cancer, (b) measuring the proportion of T cells in the first tumor sample, (c) comparing the proportion of T cells in the first tumor sample with the proportion of T cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of T cells in the first tumor sample is measured relative the proportion of T cells in the second tumor sample. In certain embodiments, the T cells are CD3+ T cells. In certain embodiments, the T cells are CD8+ T cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for managing or treating a hematological cancer comprising: (a) obtaining a first tumor sample from a first patient having the hematological cancer, (b) measuring the proportion of macrophage cells in the first tumor sample, (c) comparing the proportion of macrophage cells in the first tumor sample with the proportion of macrophage cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of macrophage cells in the first tumor sample is measured relative the proportion of macrophage cells in the second tumor sample. In certain embodiments, the macrophage cells are CD163+ macrophage cells. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are methods for managing or treating a hematological cancer comprising: (a) obtaining a first tumor sample from a first patient having the hematological cancer, (b) measuring the proportion of PD-L1+ cells in the first tumor sample, (c) comparing the proportion of PD-L1+ cells in the first tumor sample with the proportion of PD-L1+ cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of PD-L1+ cells in the first tumor sample is measured relative the proportion of PD-L1+ cells in the second tumor sample. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein are for managing or treating a hematological cancer comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and (d) administering the immunomodulatory therapy to the first patient if the proportion of the immune cells in the first tumor sample is (i) similar to the proportion of the same immune cells in tumor samples from patients having the same type of hematological cancer which are clinically sensitive to the immunomodulatory therapy, and (ii) not similar to the proportion of the same immune cells in tumor samples from patients having the same type of hematological cancer which are clinically insensitive to the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy. In a specific embodiment, the hematological cancer is diffuse large B-cell lymphoma (DLBCL). In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide. In another specific embodiment, the immunomodulatory therapy is Compound A.

In another aspect, provided herein is a kit for predicting the responsiveness of a patient having a hematological cancer to a treatment compound, comprising an agent for determining the expression levels of the genes identified in Tables 1, 2, 3, and/or 4, or a subset thereof in a biological sample obtained from the patient prior to the treatment with the treatment compound, wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, the agent is for determining the expression levels of the genes identified in Table 1. In some embodiments, the agent is for determining the expression levels of the genes identified in Table 2. In other embodiments, the agent is for determining the expression levels of the genes identified in Table 3. In some embodiments, the agent is for determining the expression levels of the genes identified in Table 4. In other embodiments, the agent is for determining the expression levels of the genes identified in Tables 1, 2, 3, and 4.

In some embodiments, the agent is for determining nucleic acid levels. In other embodiments, the agent is for determining protein levels.

### 4. BRIEF DESCRIPTION OF THE FIGURES

FIG. 1 shows Monte-Carlo cross-validation results of possible models, which demonstrate a plateau correlation of 0.95 with the outcomes using parameters that assigned non-zero weights to approximately 50 genes.

FIG. 2 shows a correlation of 0.972 using the current linear model to evaluate the validation set.

FIG. 3 shows the rescale of the current linear model.

FIGS. 4A-4D show Kaplan-Meier plots of clinical evaluation of the current linear model. FIGS. 4A and 4B demonstrate the performance in the Lenz cohort using overall survival as the outcome. FIGS. 4C and 4D demonstrate the performance in the Compound A cohort using progression free survival as the outcome. FIGS. 4A and 4C show the original 26 gene classifier. FIGS. 4B and 4D show the approximately 50 gene classifier generated from the current linear model.

FIG. 5 shows Kaplan-Meier plot for the prior 26 gene classifier in NOS-DLBCL population treated with Compound A.

FIG. 6 shows Kaplan-Meier plot for the M1 classifier (Table 1) in NOS-DLBCL population treated with Compound A.

FIG. 7 shows Kaplan-Meier plot for the M2 classifier (Table 2) in NOS-DLBCL population treated with Compound A.

FIG. 8 shows Kaplan-Meier plot for the M3 classifier (Table 3) in NOS-DLBCL population treated with Compound A.

FIGS. 9A-9D show estimated cellular proportions on Lenz cohort dataset (FIG. 9A: GSVA method; FIG. 9B: CIBERSORT method; FIG. 9C: xCell method; FIG. 9D: unmix method).

FIGS. 10A-10H show cellular proportions in tumor and immune content analysis in 26g+ and 26− DLBCL subgroups on Compound A cohort dataset (FIG. 10A: CD20+ B cells; FIG. 10B: CD3+ T cells; FIG. 10C: CD11c+ Dendritic cells; FIG. 10D: PD-L1+ cells; FIG. 10E: CD8+ T cells; FIG. 10F: CD163+ Macrophages; FIG. 10G: PD-1 total cells; FIG. 10H: CD56+NK cells).

### 5. DETAILED DESCRIPTION OF THE INVENTION

### 5.1 Terminology

The use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, or 0.05% of a given value or range.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, “hematological cancer” includes myeloma, lymphoma, and leukemia. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders, chronic myelogenous leukemia (CML), myelodysplastic syndrome (MDS), human lymphotropic virus-type 1 (HTLV-1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma.

As used herein, unless otherwise specified or indicated from context, the term “pre-treatment” as used in accordance with the methods described herein refers to prior to administration of a drug.

As used herein, unless otherwise specified or indicated from context, the terms “treatment compound,” “compound,” or “drug” refer to a compound described in Section 5.4, infra.

As used herein, the terms “patient” and “subject” refer to an animal, such as a mammal. In a specific embodiment, the patient is a human. In other embodiments, the patient is a non-human animal, such as a dog, cat, farm animal (e.g., horse, pig, or donkey), chimpanzee, or monkey. In specific embodiments, the patient is a human with a hematological cancer (e.g., DLBCL) in need of treatment.

As used herein, and unless otherwise specified, the terms “treat,” “treating” and “treatment” refer to an action that occurs while a patient is suffering from a hematological cancer (e.g., DLBCL). Treatment with a drug may results in, e.g., one, two or more of the following: reduction in the severity of the cancer, reduction in tumor size, or retardation or slowing the progression of the hematological cancer (e.g., DLBCL), increase in progression free survival, increase in time to tumor progression, and/or improvement in one or more symptoms or treatment outcomes such as complete response or partial response.

The term “complex,” when used in reference to detection of gene expression, refers to a molecule in which one or more groups are linked by bonds, which includes a non-naturally occurring molecule that is dependent upon the presence of the gene product (RNA and/or protein) in a sample. For example, a complex can include, but is not limited to, a hybridization complex, an enzyme-substrate complex, or an antibody-antigen complex. A “hybridization complex” refers to the interaction of two or more nucleic acid molecules by intermolecular forces including, but not limited to, covalent or non-covalent bonds, Van der Waals forces, dipole-dipole interactions, hydrogen bonding, and London dispersion forces.

The phrase “reaction product,” when used in reference to detection of gene expression, refers to a substance that is formed as a result of a chemical or biological reaction that is dependent upon the presence of the target substrate in a sample. In some embodiments, the reaction product is a non-naturally occurring molecule. Such a non-naturally occurring molecule can be the result of methods well-known in the art for detecting gene expression. Such methods include, but are not limited to, polymerase chain reaction (PCR) based methods including quantitative reverse transcription PCR (qRT-PCR) and reverse transcription PCR (RT-PCR), physical property assays (e.g., gel electrophoresis, Northern blot, Western blot), fluorescent in situ hybridization, serial analysis of gene expression (SAGE), Whole Transcriptome Shotgun Sequencing (WTSS), deep sequencing, microarrays/gene chips, digital gene expression analysis (e.g., NanoString assay), flow cytometry, immunofluorscence, and enzyme-linked immunosorbent assay-based methodologies (ELISA).

By “solid support,” “solid substrate” or other grammatical equivalents herein refers to any material that contains and/or can be modified to contain one or more sites (e.g., discrete individual sites, pre-defined sites, random sites, etc.) appropriate for the attachment or association of compositions disclosed herein and is amenable to at least one detection method. As will be appreciated by those in the art, there are many possible substrates. Possible substrates include, but are not limited to, glass and modified or functionalized glass, plastics (including acrylics, polystyrene and copolymers of styrene and other materials, polypropylene, polyethylene, polybutylene, polyurethanes, Teflon®, etc.), polysaccharides, nylon or nitrocellulose, resins, silica or silica-based materials (including silicon and modified silicon), carbon, metals, inorganic glasses, plastics, optical fiber bundles, and a variety of other polymers. In general, the substrates can allow optical detection and do not themselves appreciably fluoresce.

A solid support can be flat (planar), although as will be appreciated by those in the art, other configurations of substrates may be used as well; for example, three dimensional configurations can be used, for example by embedding beads in a porous block of plastic that allows sample access to the beads and using a confocal microscope for detection. Similarly, the beads may be placed on the inside surface of a tube, for flow-through sample analysis to minimize sample volume. In some aspects substrates include optical fiber bundles and flat planar substrates such as glass, polystyrene and other plastics and acrylics. A bead includes a small discrete particle, the composition of which will depend on the class of probe used and the method of synthesis. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon® may all be used. “Microsphere Detection Guide” from Bangs Laboratories, Fishers Ind. is a helpful guide.

A “label” or the phrase “detectably labeled” when used in reference to a complex or reaction product, refers to a composition that, when linked with a complex or reaction product, renders the complex or reaction product detectable, for example, by spectroscopic, photochemical, biochemical, immunochemical, or chemical means. Exemplary labels include, but are not limited to, radioactive isotopes, magnetic beads, metallic beads, colloidal particles, fluorescent dyes, enzymes, biotin, digoxigenin, haptens, and the like. A “labeled complex” or “labeled reaction product” is generally one that is bound, either covalently, through a linker or a chemical bond, or noncovalently, through ionic bonds, van der Waals forces, electrostatic attractions, hydrophobic interactions, or hydrogen bonds, to a label such that the presence of the complex or reaction product can be detected by detecting the presence of the label bound to the complex or reaction product.

As used herein, and unless otherwise specified, the term “effective amount” of a compound is an amount sufficient to provide a therapeutic benefit in the treatment of a hematological cancer (e.g., DLBCL), or to delay or minimize one or more symptoms associated with the presence of the hematological cancer (e.g., DLBCL). The term “effective amount” can encompass an amount that improves overall therapy, reduces or avoids symptoms or causes of the hematological cancer (e.g., DLBCL), or enhances the therapeutic efficacy of another therapeutic agent. In certain embodiments, an effective amount of a drug improves overall survival, disease free survival, objective response rate, time to tumor progression, progression free survival or time-to-treatment failure. In some embodiments, an effective amount of a drug results in a partial response or complete response.

The term “responsiveness” or “responsive” when used in reference to a treatment refers to the degree of effectiveness of treatment with the drug for a patient having a hematological cancer (e.g., DLBCL). For example, the term “increased responsiveness” when used in reference to treatment of a DLBCL patient can refer to an increase in the effectiveness of the drug when measured using any methods known in the art. As another example, response of a DLBCL patient to the drug can be characterized as a complete or partial response. As another example, increased responsiveness of a DLBCL patient to the drug can be characterized as overall survival, disease free survival, objective response rate, time to tumor progression, progression free survival or time-to-treatment failure. “Responsiveness” or “response” to a treatment as used herein can also refer to displaying phenotypic physical or molecular characteristics associated with a compound treatment. “Responsiveness” or “response” to a DLBCL treatment as used herein can be determined based on reduced symptoms associated with DLBCL, such as fever, weight loss, or night sweats. In some embodiments, whether a DLBCL patient is responsive to a treatment is determined by staging tests, which are tests helpful for determining which areas of the body have been affected by follicular lymphoma. Such tests include, but not limited to, blood tests, bone marrow biopsy, CT scan, and PET/CT scan. Staging involves dividing patients into groups (stages) based upon how much of the lymphatic system is involved. Stages of lymphoma can be defined as follows: stage I—only one lymph node region is involved, only one lymph structure is involved, or only one extranodal site (IE) is involved; stage II—two or more lymph node regions or lymph node structures on the same side of the diaphragm are involved; stage III—lymph node regions or structures on both sides of the diaphragm are involved; stage IV—there is widespread involvement of a number of organs or tissues other than lymph node regions or structures, such as the liver, lung, or bone marrow. A lymph node region refers to an area of lymph nodes and the surrounding tissue, e.g., the cervical nodes in the neck, the axillary nodes in the armpit, the inguinal nodes in the groin, and the mediastinal nodes in the chest. A lymph structure refers to an organ or a structure that is part of the lymphatic system, such as the lymph nodes, spleen, and thymus gland.

“Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in any measurable tumor burden (i.e., the number of malignant cells present in the subject, or the measured bulk of tumor masses or the quantity of abnormal monoclonal protein) in the absence of new lesions.

“Overall survival” is defined as the time from randomization until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval.

Several endpoints are based on tumor assessments. These endpoints include disease free survival (DFS), objective response rate (ORR), time to progression (TTP), progression-free survival (PFS), and time-to-treatment failure (TTF). The collection and analysis of data on these time-dependent endpoints are based on indirect assessments, calculations, and estimates (e.g., tumor measurements).

Generally, “disease free survival” (DFS) is defined as the time from randomization until recurrence of tumor or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior tumor progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

“Objective response rate” (ORR) is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. If available, standardized criteria should be used to ascertain response. A variety of response criteria have been considered appropriate (e.g., RECIST criteria) (Therasse et al., (2000) J. Natl. Cancer Inst, 92: 205-16). The significance of ORR is assessed by its magnitude and duration, and the percentage of complete responses (no detectable evidence of tumor).

“Time to progression” (TTP) and “progression-free survival” (PFS) have served as primary endpoints for drug approval. TTP is defined as the time from randomization until objective tumor progression; TTP does not include deaths. PFS is defined as the time from randomization until objective tumor progression or death. Compared with TTP, PFS is the preferred regulatory endpoint. PFS includes deaths and thus can be a better correlate to overall survival. PFS assumes patient deaths are randomly related to tumor progression. However, in situations where the majority of deaths are unrelated to cancer, TTP can be an acceptable endpoint.

As an endpoint to support drug approval, PFS can reflect tumor growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

“Time-to-treatment failure” (TTF) is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient or physician withdrawal, or patient intolerance.

The term “similar” or an equivalent thereof as used herein refers to resembling the reference or comparative term. In some embodiments, a numerical value is the value similar to a reference value if the difference between the numerical value and the reference value is within 5%, 10%, 15%, 20%, or 25% of the reference value. In some embodiments, a numerical value is similar to a reference value if the difference between the numerical value and the reference value is within 0.5% to 5%, 5% to 10%, 10% to 20%, or 15% to 25% of the reference value. In other embodiments, the term “similar” or an equivalent thereof is determined based on multiple features (or values) of a sample, for example, the expression levels of multiple genes of a patient. In yet other embodiments, the term “similar” or an equivalent thereof refers to belonging to the same group as determined by a classifier. In some embodiments, the term “similar” or an equivalent thereof is a relative term. For example, the similarity of a sample to a first reference sample is determined by comparing the sample with the first reference sample and a second reference sample, i.e., the sample is similar to the first reference sample as compared with the second reference sample.

The term “not similar,” “differential,” or an equivalent thereof as used herein refers to not resembling the reference or comparative term. In certain embodiments, a numerical value is the value not similar or differential to a reference value if the difference between the numerical value and the reference value is greater than 25% of the reference value. In other embodiments, the term “not similar,” “differential,” or an equivalent thereof is determined based on multiple features (or values) of a sample, for example, the expression levels of multiple genes of a patient. In yet other embodiments, the term “not similar,” “differential,” or an equivalent thereof refers to not belonging to the same group as determined by a classifier. In some embodiments, the term “not similar,” “differential,” or an equivalent thereof is a relative term. For example, a sample is not similar to a first reference sample is determined by comparing the sample with the first reference sample and a second reference sample, i.e., the sample is not similar to the first reference sample as compared with the second reference sample.

The term “predict” generally means to determine or tell in advance. When used to “predict” the effectiveness of a drug to treat a patient having a hematological cancer (e.g., DLBCL), the term can mean, for example, the likelihood that the patient having the hematological cancer (e.g., DLBCL) will be responsive to the drug, as assessed prior to treatment with the drug or within a short period (e.g., within in hours, 1, 2, 3, 4 or more days, 1 week, or 2 weeks) of the treatment with the drug has begun.

As used herein the terms “polypeptide” and “protein” as used interchangeably herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. The polypeptide can be purified from a biological sample.

An mRNA that is “upregulated” is generally increased upon a given treatment or condition. An mRNA that is “downregulated” generally refers to a decrease in the level of expression of the mRNA in response to a given treatment or condition. In some situations, the mRNA level can remain unchanged upon a given treatment or condition.

An mRNA from a patient sample can be “upregulated” when treated with a drug, as compared to a control. This upregulation can be, for example, an increase of about 5%, 10%, 20%, 30%, 40%, 50%, 60%, 70%, 90%, 100%, 200%, 300%, 500%, 1,000%, 5,000% or more of the comparative control mRNA level.

Alternatively, an mRNA can be “downregulated”, or expressed at a lower level, in response to administration of certain drug or other therapies. A downregulated mRNA can be, for example, present at a level of about 99%, 95%, 90%, 80%, 70%, 60%, 50%, 40%, 30%, 20%, 10%, 1% or less of the comparative control mRNA level.

Similarly, a protein that is “upregulated” is generally increased upon a given treatment or condition. A protein that is “downregulated” generally refers to a decrease in the level of the protein in response to a given treatment or condition. In some situations, the protein level can remain unchanged upon a given treatment or condition.

The level of a polypeptide or protein biomarker from a patient sample can be increased when treated with a drug, as compared to a non-treated control. This increase can be about 5%, 10%, 20%, 30%, 40%, 50%, 60%, 70%, 90%, 100%, 200%, 300%, 500%, 1,000%, 5,000% or more of the comparative control protein level.

Alternatively, the level of a protein biomarker can be decreased in response to administration of certain drugs/compounds or other agents. This decrease can be, for example, present at a level of about 99%, 95%, 90%, 80%, 70%, 60%, 50%, 40%, 30%, 20%, 10%, 1% or less of the comparative control protein level.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. A nucleic acid also includes, in some aspects, a complementary DNA (cDNA). The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The terms “isolated” and “purified” refer to isolation of a substance (such as mRNA or protein) such that the substance comprises a substantial portion of the sample in which it resides, i.e. greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

The term “sample” as used herein relates to a material or mixture of materials, typically, although not necessarily, in fluid form, containing one or more elements of interest.

The term “sample,” as used herein, can be a biological sample or a synthetically derived sample.

“Biological sample” as used herein refers to a sample obtained from a biological subject, including sample of biological tissue or fluid origin, obtained, reached, or collected in vivo or in situ. A biological sample also includes samples from a region of a patient containing precancerous or cancer cells or tissues. Such samples can be, but are not limited to, organs, tissues, fractions and cells isolated from a patient. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Other exemplary biological samples include whole blood, partially purified blood, PBMCs, tissue biopsies, and the like. In a specific embodiment, the biological sample is a tumor biopsy.

A “classifier” refers to a mathematical function that separates two or more groups, bodies or distributions of data according to the values with which each example of data is described, or an output of a function applied to those values in a manner that may be used to assign new data examples to one or more of the groups, bodies or distributions separated. The term “classifier” as used herein also includes regression or a function on data values that maps to a continuous target variable associated with the data examples, such as a function that relates gene expression values to PFS.

A biological marker or “biomarker” is a substance whose detection indicates a particular biological state, such as, for example, the responsiveness of a patient having a hematological cancer (e.g., DLBCL) to treatment with a drug. In some embodiments, biomarkers can either be measured individually, or several biomarkers can be measured simultaneously.

In some embodiments, a “biomarker” indicates a change in the level of mRNA expression that may correlate with responsiveness of a patient having a hematological cancer (e.g., DLBCL) to treatment with a drug. In some embodiments, the biomarker is a nucleic acid, such as an mRNA or cDNA.

In additional embodiments, a “biomarker” indicates a change in the level of polypeptide or protein expression that may correlate with the responsiveness of a patient having a hematological cancer (e.g., DLBCL) to treatment with a drug. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof, which further includes a stoichiometric or non-stoichiometric amount of solvent bound by non-covalent intermolecular forces. Where the solvent is water, the solvate is a hydrate.

As used herein the term “enantiomer,” “isomer” or “stereoisomer” encompasses all enantiomerically/stereomerically pure and enantiomerically/stereomerically enriched compounds provided herein.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

As used herein the term “polymorph” means solid crystalline forms of a compound provided herein or complex thereof. Different polymorphs of the same compound can exhibit different physical, chemical and/or spectroscopic properties.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al. (1989) Molecular Cloning; A Laboratory Manual (2d ed.); D. N Glover, ed. (1985) DNA Cloning, Volumes I and II; M. J. Gait, ed. (1984) Oligonucleotide Synthesis; B. D. Hames & SJ. Higgins, eds. (1984) Nucleic Acid Hybridization; B. D. Hames & S. J. Higgins, eds. (1984) Transcription and Translation; R. I. Freshney, ed. (1986) Animal Cell Culture; Immobilized Cells and Enzymes (IRL Press, 1986); Immunochemical Methods in Cell and Molecular Biology (Academic Press, London); Scopes (1987) Protein Purification: Principles and Practice (2d ed.; Springer Verlag, N.Y.); and D. M. Weir and C. C. Blackwell, eds. (1986) Handbook of Experimental Immunology, Volumes I-IV.

### 5.2 Methods for Predicting Responsiveness of Patients Having a Hematological Cancer (e.g., DLBCL) to Treatment Compounds

The present application is based, in part, on the finding that the expression levels of several groups of genes (e.g., those listed in Tables 1, 2, 3, and 4) correlate with responsiveness of a patient having a hematological cancer (e.g., DLBCL) to certain treatment compounds, such as lenalidomide or Compound A. Thus, in one aspect, provided herein are methods that can use the biomarkers and/or classifiers identified in Tables 1, 2, 3, and 4 below to predict responsiveness of a patient having a hematological cancer (e.g., DLBCL) to a drug, such as lenalidomide or Compound A.

TABLE 1
Gene

BICC1
C10orf54
C1RL
C20orf112
C8orf4
CCDC88C
CD97
CEBPB
CFB
CHRDL1
CIRH1A
CPSF3
CSF1R
DDR2
DLGAP5
DUSP1
EMR2
EPB41L3
FBXO32
GAS2L1
GINS4
IFITM2
KCNMB1
LGALS3BP
LRP11
MEGF6
MEIS1
MYO1F
PDGFC
PRNP
PTPRM
RAB31
RBBP8
RUVBL1
SEMA3C
SERPING1
SMOC2
SNRPD1
TBC1D2B
THEMIS2
TMEM173
TPSAB1
TRIP13
TXNIP
ULK1
WDR12
WWTR1
XAF1
ZNF215

TABLE 2
Gene

ALKBH2
AOAH
C10orf54
C1RL
C8orf4
CD84
CD97
CDC20
CEBPB
CEBPD
CHRDL1
CIRH1A
CPSF3
CYP1B1
FBXO32
FOSL2
GAS2L1
GBP3
GINS4
GNAQ
GPR155
GPRIN3
HJURP
KCNMB1
LRP11
MAFB
MEGF6
MEIS1
MYO1F
MYOF
PCSK5
PDGFC
PHACTR2
PTPRM
RAB31
RALGDS
RUVBL1
SASH1
SERPING1
SMOC2
SNRPD1
SSH1
TBC1D2B
TMEM173
TP53INP2
TRIP13
ULK1
WWTR1
XAF1
ZNF215

TABLE 3
Gene

C10orf54
C1RL
C20orf112
C8orf4
CCDC88C
CILP
CIRH1A
CLU
CPVL
CSF1R
CTSB
EPB41L3
FBXO32
IFI44
LRP11
MEGF6
MEIS1
PLAT
SERPING1
SPC25
THEMIS2
TPSAB1
ULK1
XAF1
ZNF215

TABLE 4
Gene

C10orf54
C1RL
C20orf112
C8orf4
CCNB1
CDC6
CILP
CLU
CPSF3
CPVL
CSF1R
CTSB
EPB41L3
FBXO32
IFI44
MEIS1
PHACTR2
PLAT
RUVBL1
SERPING1
SNRPD1
TRIP13
XAF1
ZNF215

In some embodiments, provided herein is a method for predicting the responsiveness of a patient having a hematological cancer to a treatment compound, comprising:

(a) obtaining a biological sample from the patient having the hematological cancer;

(b) determining the expression level of one, two, three, four, five, or more of the genes identified in Tables 1, 2, 3, and/or 4; and

(c) comparing the expression level of the one, two, three, four, five, or more of the genes identified in Tables 1, 2, 3, and/or 4 in the biological sample with a reference expression level of the same genes;

wherein the differential expression level of the one, two, three, four, five, or more of the genes in the biological sample relative to the reference expression level of the one, two, three, four, five, or more of the genes indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In certain embodiments, the reference expression level of the one, two, three, four, five, or more of the genes identified in Tables 1, 2, 3, and/or 4 is the expression level of the same genes in a reference sample. In one embodiment, the reference sample is a second biological sample. In another embodiment, the reference sample is synthetically derived.

In some embodiments, the expression levels of all genes identified in Tables 1, 2, 3, or 4 is determined. In some embodiments, the expression levels of all genes identified in Table 1 is determined. In some embodiments, the expression levels of all genes identified in Table 2 is determined. In some embodiments, the expression levels of all genes identified in Table 3 is determined. In some embodiments, the expression levels of all genes identified in Table 4 is determined.

In a specific embodiment, provided herein is a method for predicting the responsiveness of a patient having a hematological cancer to a treatment compound, comprising:

(a) obtaining a biological sample from the patient having the hematological cancer;

(b) determining the expression levels of all the genes identified in Table 1; and

(c) comparing the expression levels of the genes identified in Table 1 in the biological sample with reference expression levels of the same genes;

wherein the differential expression levels of the genes identified in Table 1 in the biological sample relative to the reference expression levels of the genes identified in Table 1 indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In another specific embodiment, provided herein is a method for predicting the responsiveness of a patient having a hematological cancer to a treatment compound, comprising:

(a) obtaining a biological sample from the patient having the hematological cancer;

(b) determining the expression levels of all the genes identified in Table 2; and

(c) comparing the expression levels of the genes identified in Table 2 in the biological sample with reference expression levels of the same genes;

wherein the differential expression levels of the genes identified in Table 2 in the biological sample relative to the reference expression levels of the genes identified in Table 2 indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a biological sample from the patient having the hematological cancer;

(b) determining the expression levels of all the genes identified in Table 3; and

(c) comparing the expression levels of the genes identified in Table 3 in the biological sample with reference expression levels of the same genes;

wherein the differential expression levels of the genes identified in Table 3 in the biological sample relative to the reference expression levels of the genes identified in Table 3 indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a biological sample from the patient having the hematological cancer;

(b) determining the expression levels of all the genes identified in Table 4; and

(c) comparing the expression levels of the genes identified in Table 4 in the biological sample with reference expression levels of the same genes;

wherein the differential expression levels of the genes identified in Table 4 in the biological sample relative to the reference expression levels of the genes identified in Table 4 indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. In certain embodiments, provided herein is a method for predicting the responsiveness of a patient having a hematological cancer to a treatment compound, comprising:

(a) obtaining a first biological sample from a first patient having the hematological cancer;

(b) determining the expression level of one, two, three, four, five, or more of the genes identified in Tables 1, 2, 3, and/or 4; and

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is not responsive to the treatment compound,

wherein the differential expression level of the one, two, three, four, five, or more of the genes in the first biological sample relative to the expression level of the one, two, three, four, five, or more of the genes in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, provided herein is a method for predicting the responsiveness of a patient having a hematological cancer to a treatment compound, comprising:

(a) obtaining a first biological sample from a first patient having the hematological cancer;

(b) determining the expression level of one, two, three, four, five, or more of the genes identified in Tables 1, 2, 3, and/or 4; and

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is responsive to the drug,

wherein the similar expression level of the one, two, three, four, five, or more of the genes in the first biological sample relative to the expression level of the one, two, three, four, five, or more of the genes in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, the expression levels of all genes identified in Tables 1, 2, 3, or 4 is determined. In some embodiments, the expression levels of all genes identified in Table 1 is determined. In some embodiments, the expression levels of all genes identified in Table 2 is determined. In some embodiments, the expression levels of all genes identified in Table 3 is determined. In some embodiments, the expression levels of all genes identified in Table 4 is determined.

In a specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 1; and

(c) comparing the expression levels of the genes identified in Table 1 in the first biological sample with the expression levels of the same genes in a second biological sample obtained from a second patient having the hematological cancer, wherein the second patient is not responsive to the treatment compound,

wherein the differential expression levels of the genes identified in Table 1 in the first biological sample relative to the expression levels of the genes identified in Table 1 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 1; and

(c) comparing the expression levels of the genes identified in Table 1 in the first biological sample with the expression levels of the same genes in a second biological sample obtained from a second patient having the hematological cancer, wherein the second patient is responsive to the drug,

wherein the similar expression levels of the genes identified in Table 1 in the first biological sample relative to the expression levels of the genes identified in Table 1 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 2; and

(c) comparing the expression levels of the genes identified in Table 2 in the first biological sample with the expression levels of the same genes in a second biological sample obtained from a second patient having the hematological cancer, wherein the second patient is not responsive to the treatment compound,

wherein the differential expression levels of the genes identified in Table 2 in the first biological sample relative to the expression levels of the genes identified in Table 2 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 2; and

(c) comparing the expression levels of the genes identified in Table 2 in the first biological sample with the expression levels of the same genes in a second biological sample obtained from a second patient having the hematological cancer, wherein the second patient is responsive to the drug,

wherein the similar expression levels of the genes identified in Table 2 in the first biological sample relative to the expression levels of the genes identified in Table 2 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 3; and

(c) comparing the expression levels of the genes identified in Table 3 in the first biological sample with the expression levels of the same genes in a second biological sample obtained from a second patient having the hematological cancer, wherein the second patient is not responsive to the treatment compound,

wherein the differential expression levels of the genes identified in Table 3 in the first biological sample relative to the expression levels of the genes identified in Table 3 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 3; and

(c) comparing the expression levels of the genes identified in Table 3 in the first biological sample with the expression levels of the same genes in a second biological sample obtained from a second patient having the hematological cancer, wherein the second patient is responsive to the drug,

wherein the similar expression levels of the genes identified in Table 3 in the first biological sample relative to the expression levels of the genes identified in Table 3 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 4; and

(c) comparing the expression levels of the genes identified in Table 4 in the first biological sample with the expression levels of the same genes in a second biological sample obtained from a second patient having the hematological cancer, wherein the second patient is not responsive to the treatment compound,

wherein the differential expression levels of the genes identified in Table 4 in the first biological sample relative to the expression levels of the genes identified in Table 4 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of all genes identified in Table 4; and

(c) comparing the expression levels of the genes identified in Table 4 in the first biological sample with the expression levels of the same genes in a second biological sample obtained from a second patient having the hematological cancer, wherein the second patient is responsive to the drug,

wherein the similar expression levels of the genes identified in Table 4 in the first biological sample relative to the expression levels of the genes identified in Table 4 in the second biological sample indicates that the first patient will be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In other embodiments, provided herein is a method for predicting the responsiveness of a patient having a hematological cancer to a treatment compound, comprising:

(a) obtaining a first biological sample from a first patient having the hematological cancer;

(b) determining the expression level of one, two, three, four, five, or more of the genes identified in Tables 1, 2, 3, and/or 4; and

(c) comparing the gene expression profile of the genes or subset of genes in the first biological sample to
(i) the gene expression profile of the one, two, three, four, five, or more of the genes in a second biological sample from a second patient having the hematological cancer, wherein the second patient is responsive to the treatment compound, and
(ii) the gene expression profile of the one, two, three, four, five, or more of the genes in a third biological sample from a third patient having the hematological cancer, wherein the third patient is not responsive to the treatment compound,

wherein the similar expression level of the one, two, three, four, five, or more of the genes in the first biological sample relative to the expression level of the one, two, three, four, five, or more of the genes in the second biological sample indicates that the first patient will be responsive to the treatment compound, and the similar expression level of the one, two, three, four, five, or more of the genes in the first biological sample relative to the expression level of the one, two, three, four, five, or more of the genes in the third biological sample indicates that the first patient will not be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, the one, two, three, four, five, or more of genes are a subset of the genes in Table 1, a subset of the genes in Table 2, a subset of the genes in Table 3, and/or a subset of the genes in Table 4. In other embodiments, the one, two, three, four, five, or more of genes are a combination of a subset of the genes in two tables selected from Tables 1-4. In yet other embodiments, the one, two, three, four, five, or more of genes are a combination of a subset of the genes in three tables selected from Tables 1-4. In still other embodiments, the one, two, three, four, five, or more of genes are a combination of a subset of the genes in Table 1, a subset of the genes in Table 2, a subset of the genes in Table 3, and a subset of the genes in Table 4.

In some embodiments, the expression levels of all genes identified in Tables 1, 2, 3, or 4 is determined. In some embodiments, the expression levels of all genes identified in Table 1 is determined. In some embodiments, the expression levels of all genes identified in Table 2 is determined. In some embodiments, the expression levels of all genes identified in Table 3 is determined. In some embodiments, the expression levels of all genes identified in Table 4 is determined.

In a specific embodiment, provided herein is a method for predicting the responsiveness of a patient having a hematological cancer to a treatment compound, comprising:

(a) obtaining a first biological sample from a first patient having the hematological cancer;

(b) determining the expression levels of genes identified in Table 1; and

(c) comparing the gene expression profile of the genes identified in Table 1 in the first biological sample to
(i) the gene expression profile of the genes identified in Table 1 in a second biological sample from a second patient having the hematological cancer, wherein the second patient is responsive to the treatment compound, and
(ii) the gene expression profile of the genes identified in Table 1 in a third biological sample from a third patient having the hematological cancer, wherein the third patient is not responsive to the treatment compound,
   wherein the similar expression levels of the genes in the first biological sample relative to the expression levels of the genes in the second biological sample indicates that the first patient will be responsive to the treatment compound, and the similar expression levels of the genes in the first biological sample relative to the expression levels of the genes in the third biological sample indicates that the first patient will not be responsive to the treatment compound, and wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In another specific embodiment, provided herein is a method for predicting the responsiveness of a patient having a hematological cancer to a treatment compound, comprising:

(a) obtaining a first biological sample from a first patient having the hematological cancer;

(b) determining the expression levels of genes identified in Table 2; and

(c) comparing the gene expression profile of the genes identified in Table 2 in the first biological sample to
(i) the gene expression profile of the genes identified in Table 2 in a second biological sample from a second patient having the hematological cancer, wherein the second patient is responsive to the treatment compound, and
(ii) the gene expression profile of the genes identified in Table 2 in a third biological sample from a third patient having the hematological cancer, wherein the third patient is not responsive to the treatment compound,

wherein the similar expression levels of the genes in the first biological sample relative to the expression levels of the genes in the second biological sample indicates that the first patient will be responsive to the treatment compound, and the similar expression levels of the genes in the first biological sample relative to the expression levels of the genes in the third biological sample indicates that the first patient will not be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of genes identified in Table 3; and

(c) comparing the gene expression profile of the genes identified in Table 3 in the first biological sample to
(i) the gene expression profile of the genes identified in Table 3 in a second biological sample from a second patient having the hematological cancer, wherein the second patient is responsive to the treatment compound, and
(ii) the gene expression profile of the genes identified in Table 3 in a third biological sample from a third patient having the hematological cancer, wherein the third patient is not responsive to the treatment compound,

wherein the similar expression levels of the genes in the first biological sample relative to the expression levels of the genes in the second biological sample indicates that the first patient will be responsive to the treatment compound, and the similar expression levels of the genes in the first biological sample relative to the expression levels of the genes in the third biological sample indicates that the first patient will not be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In yet another specific embodiment, provided herein is a method for predicting the responsiveness of a patient having a hematological cancer to a treatment compound, comprising:

(a) obtaining a first biological sample from a first patient having the hematological cancer;

(b) determining the expression levels of genes identified in Table 4; and

(c) comparing the gene expression profile of the genes identified in Table 4 in the first biological sample to
(i) the gene expression profile of the genes identified in Table 4 in a second biological sample from a second patient having the hematological cancer, wherein the second patient is responsive to the treatment compound, and
(ii) the gene expression profile of the genes identified in Table 4 in a third biological sample from a third patient having the hematological cancer, wherein the third patient is not responsive to the treatment compound,

wherein the similar expression levels of the genes in the first biological sample relative to the expression levels of the genes in the second biological sample indicates that the first patient will be responsive to the treatment compound, and the similar expression levels of the genes in the first biological sample relative to the expression levels of the genes in the third biological sample indicates that the first patient will not be responsive to the treatment compound, and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, expression levels of multiple genes (biomarkers) provided herein are used to predict a patient's response to the compound provided herein. When expression levels of multiple genes (biomarkers) are used to predict a patient's response to a treatment with the compound provided herein, e.g., lenalidomide or Compound A, any classifier that classifies based on two or more features can be used in the present disclosure. For example, in some embodiments, a population of DLBCL patients that have received a treatment with the compound provide herein (e.g., lenalidomide or Compound A) are divided into two groups based on their responsiveness to the compound treatment, i.e., responsive patient group and non-responsive patient group. The expression levels of two or more genes provided herein (e.g., those listed in Tables 1-4) for each patient in the population are analyzed using a classifier and a score can be generated. A reference score or a threshold score can be generated based on the scores of the responsive patients and the scores of non-responsive patients. Such reference score can be used to predict a patient's responsiveness to the compound based on the expression levels of the genes of this patient.

In some embodiments, the methods provided herein further comprise (a) generating a score of the sample based on the expression levels of the genes or a subset thereof provided herein in the sample; and (b) determining the probability of the subject being responsive to the compound provided herein by comparing the score of the sample to a reference score.

In some embodiments, the methods provided herein further comprise: (a) determining the expression levels of the genes or a subset thereof provided herein in a biological sample from a population of subjects that previously have been administered with the compound provided herein, (b) generating a score for the expression levels of the genes or a subset thereof provided herein for each subject of the population; (c) differentiating the subjects that are responsive to the compound provided herein from those subjects that are not responsive to the compound provided herein; and (d) determining responsiveness to the compound provided herein based on the scores for the subjects that are responsive to the compound and those subjects that are not responsive to the compound.

In other embodiments, the methods provided herein further comprise: (a) determining the expression levels of the genes or a subset thereof provided herein in a biological sample from a population of subjects that previously have been administered with the compound, (b) generating a score for the expression levels of the genes or a subset thereof provided herein for each subject of the population; (c) differentiating the subjects that are responsive to the compound provided herein from those subjects that are not responsive to compound provided herein; and (d) generating a reference score that is predictive of the responsiveness of a subject to the compound provided herein using a model based on the scores for the subjects that are responsive to the compound and those subjects that are not responsive to the compound.

In another aspect, provided herein are methods for predicting the responsiveness of a patient having a hematological cancer to a treatment compound, comprising: (a) obtaining a biological sample from a patient having the hematological cancer; (b) determining the expression of the genes or a certain subset of genes set forth in Tables 1, 2, 3 and/or 4, or any combination thereof, in the biological sample and generating a score based on the expression of the genes or subset of genes, (c) comparing the score to a reference score, wherein a score similar to a reference score indicates that the patient will be responsive to the treatment compound. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In some embodiments, the expression levels of all genes identified in Tables 1, 2, 3, or 4 is determined. In some embodiments, the expression levels of all genes identified in Table 1 is determined. In some embodiments, the expression levels of all genes identified in Table 2 is determined. In some embodiments, the expression levels of all genes identified in Table 3 is determined. In some embodiments, the expression levels of all genes identified in Table 4 is determined.

In a specific embodiment, provided herein is a method for predicting the responsiveness of a patient having a hematological cancer to a treatment compound, comprising: (a) obtaining a biological sample from a patient having the hematological cancer; (b) determining the expression of the genes identified in Table 1 in the biological sample and generating a score based on the expression of the genes, (c) comparing the score to a reference score, wherein a score similar to a reference score indicates that the patient will be responsive to the treatment compound. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In another specific embodiment, provided herein is a method for predicting the responsiveness of a patient having a hematological cancer to a treatment compound, comprising: (a) obtaining a biological sample from a patient having the hematological cancer; (b) determining the expression of the genes identified in Table 2 in the biological sample and generating a score based on the expression of the genes, (c) comparing the score to a reference score, wherein a score similar to a reference score indicates that the patient will be responsive to the treatment compound. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In a specific embodiment, provided herein is a method for predicting the responsiveness of a patient having a hematological cancer to a treatment compound, comprising: (a) obtaining a biological sample from a patient having the hematological cancer; (b) determining the expression of the genes identified in Table 3 in the biological sample and generating a score based on the expression of the genes, (c) comparing the score to a reference score, wherein a score similar to a reference score indicates that the patient will be responsive to the treatment compound. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In a specific embodiment, provided herein is a method for predicting the responsiveness of a patient having a hematological cancer to a treatment compound, comprising: (a) obtaining a biological sample from a patient having the hematological cancer; (b) determining the expression of the genes identified in Table 4 in the biological sample and generating a score based on the expression of the genes, (c) comparing the score to a reference score, wherein a score similar to a reference score indicates that the patient will be responsive to the treatment compound. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In another aspect, provided herein are methods for predicting the responsiveness of a patient having a hematological cancer to a treatment compound, comprising: (a) obtaining a biological sample from a patient having the hematological cancer; (b) determining the expression of the genes or a certain subset of genes set forth in Tables 1, 2, 3, and/or 4, or any combination thereof, in the biological sample and generating a score based on the expression of the genes or subset of genes, (c) comparing the score to a first reference score and a second reference score, wherein (i) the score similar to the first reference score indicates that the patient will be responsive to the treatment compound, and/or (ii) the score similar to the second reference score indicates that the patient will be responsive to the treatment compound. In specific embodiments, the first reference score is based on the expression of the same genes or subset of genes in a population of patients having the hematological cancer who are responsive to the treatment compound, and the second reference score is based on the expression of the same genes or subset of genes in a population of patients having the hematological cancer who are not responsive to the treatment compound. In a specific embodiment, the treatment compound is a compound disclosed herein. In one embodiment, the treatment compound is lenalidomide. In another embodiment, the treatment compound is Compound A.

In some embodiments, the expression levels of all genes identified in Tables 1, 2, 3, or 4 is determined. In some embodiments, the expression levels of all genes identified in Table 1 is determined. In some embodiments, the expression levels of all genes identified in Table 2 is determined. In some embodiments, the expression levels of all genes identified in Table 3 is determined. In some embodiments, the expression levels of all genes identified in Table 4 is determined.

In a specific embodiment, provided herein is a method for predicting the responsiveness of a patient having a hematological cancer to a treatment compound, comprising: (a) obtaining a biological sample from a patient having the hematological cancer; (b) determining the expression of genes identified in Table 1 in the biological sample and generating a score based on the expression of the genes, (c) comparing the score to a first reference score and a second reference score, wherein (i) the score similar to the first reference score indicates that the patient will be responsive to the treatment compound, and/or (ii) the score similar to the second reference score indicates that the patient will be responsive to the treatment compound, wherein the first reference score is based on the expression of the same genes in a population of patients having the hematological cancer who are responsive to the treatment compound, and the second reference score is based on the expression of the same genes in a population of patients having the hematological cancer who are not responsive to the treatment compound, wherein the treatment compound the treatment compound is lenalidomide or Compound A.

In another specific embodiment, provided herein is a method for predicting the responsiveness of a patient having a hematological cancer to a treatment compound, comprising: (a) obtaining a biological sample from a patient having the hematological cancer; (b) determining the expression of genes identified in Table 2 in the biological sample and generating a score based on the expression of the genes, (c) comparing the score to a first reference score and a second reference score, wherein (i) the score similar to the first reference score indicates that the patient will be responsive to the treatment compound, and/or (ii) the score similar to the second reference score indicates that the patient will be responsive to the treatment compound, wherein the first reference score is based on the expression of the same genes in a population of patients having the hematological cancer who are responsive to the treatment compound, and the second reference score is based on the expression of the same genes in a population of patients having the hematological cancer who are not responsive to the treatment compound, wherein the treatment compound the treatment compound is lenalidomide or Compound A.

In yet another specific embodiment, provided herein is a method for predicting the responsiveness of a patient having a hematological cancer to a treatment compound, comprising: (a) obtaining a biological sample from a patient having the hematological cancer; (b) determining the expression of genes identified in Table 3 in the biological sample and generating a score based on the expression of the genes, (c) comparing the score to a first reference score and a second reference score, wherein (i) the score similar to the first reference score indicates that the patient will be responsive to the treatment compound, and/or (ii) the score similar to the second reference score indicates that the patient will be responsive to the treatment compound, wherein the first reference score is based on the expression of the same genes in a population of patients having the hematological cancer who are responsive to the treatment compound, and the second reference score is based on the expression of the same genes in a population of patients having the hematological cancer who are not responsive to the treatment compound, wherein the treatment compound the treatment compound is lenalidomide or Compound A.

In yet another specific embodiment, provided herein is a method for predicting the responsiveness of a patient having a hematological cancer to a treatment compound, comprising: (a) obtaining a biological sample from a patient having the hematological cancer; (b) determining the expression of genes identified in Table 4 in the biological sample and generating a score based on the expression of the genes, (c) comparing the score to a first reference score and a second reference score, wherein (i) the score similar to the first reference score indicates that the patient will be responsive to the treatment compound, and/or (ii) the score similar to the second reference score indicates that the patient will be responsive to the treatment compound, wherein the first reference score is based on the expression of the same genes in a population of patients having the hematological cancer who are responsive to the treatment compound, and the second reference score is based on the expression of the same genes in a population of patients having the hematological cancer who are not responsive to the treatment compound, wherein the treatment compound the treatment compound is lenalidomide or Compound A.

In some embodiments, the determining step of the methods described herein comprising determining the expression of the genes or a certain subset of genes set forth in Table 1 in the first biological sample. For example, in certain embodiments, the subset of genes can include 2, 3, 4, 5, 6, 7, 8, 9, 10, 11, 12, 13, 14, 15, 16, 20, 25, 30, 35, 40, 45, or more genes listed in Table 1. In another embodiment, the subset of genes can include between 2 to 40 genes, alternatively between 3 to 45 genes, alternatively between 5 to 15 genes, or alternatively between 10 to 20 genes listed in Table 1. In some embodiments, the determining step of the methods described herein comprises determining the expression of one or more genes selected from the group consisting of BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, and ZNF215.

In some embodiments of the various methods provided herein, the determining step of the methods described herein comprises determining the expression of all the genes listed in Table 1—BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, and ZNF215.

In some embodiments, the determining step of the methods described herein comprising determining the expression of the genes or a certain subset of genes set forth in Table 2 in the first biological sample. For example, in certain embodiments, the subset of genes can include 2, 3, 4, 5, 6, 7, 8, 9, 10, 11, 12, 13, 14, 15, 16, 20, 25, 30, 35, 40, 45, or more genes listed in Table 2. In another embodiment, the subset of genes can include between 2 to 40 genes, or alternatively between 3 to 45 genes, alternatively between 5 to 15 genes, or alternatively 10 to 20 genes listed in Table 2. In some embodiments, the determining step of the methods described herein comprises determining the expression of one or more genes selected from the group consisting of ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, and ZNF215.

In some embodiments of the various methods provided herein, the determining step of the methods described herein comprises determining the expression of all the genes listed in Table 2—ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, and ZNF215.

In some embodiments, the determining step of the methods described herein comprising determining the expression of the genes or a certain subset of genes set forth in Table 3 in the first biological sample. For example, in certain embodiments, the subset of genes can include 2, 3, 4, 5, 6, 7, 8, 9, 10, 11, 12, 13, 14, 15, 16, 20, or more genes listed in Table 3. In another embodiment, the subset of genes can include between 2 to 20 genes, or alternatively between 3 to 24 genes, alternatively between 5 to 15 genes, or alternatively 10 to 24 genes listed in Table 3. In some embodiments, the determining step of the methods described herein comprises determining the expression of one or more genes selected from the group consisting of C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, and ZNF215.

In some embodiments of the various methods provided herein, the determining step of the methods described herein comprises determining the expression of all the genes listed in Table 3—C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, and ZNF215.

In some embodiments, the determining step of the methods described herein comprising determining the expression of the genes or a certain subset of genes set forth in Table 4 in the first biological sample. For example, in certain embodiments, the subset of genes can include 2, 3, 4, 5, 6, 7, 8, 9, 10, 11, 12, 13, 14, 15, 16, 20, or more genes listed in Table 4. In another embodiment, the subset of genes can include between 2 to 20 genes, or alternatively between 3 to 24 genes, alternatively between 5 to 15 genes, or alternatively 10 to 24 genes listed in Table 4. In some embodiments, the determining step of the methods described herein comprises determining the expression of one or more genes selected from the group consisting of C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, and ZNF215.

In some embodiments of the various methods provided herein, the determining step of the methods described herein comprises determining the expression of all the genes listed in Table 4—C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, and ZNF215.

In some embodiments, the determining step of the methods described herein comprising determining the expression of the genes or a certain subset of genes set forth in a combination of Tables 1, 2, 3, and 4 in the first biological sample. For example, in certain embodiments, the subset of genes can include 2, 3, 4, 5, 6, 7, 8, 9, 10, 11, 12, 13, 14, 15, 16, 20, 25, 30, 35, 40, 50, 60, 70, or more genes listed in Tables 1, 2, 3, and 4. In another embodiment, the subset of genes can include between 2 to 71 genes, alternatively between 2 to 20 genes, alternatively between 3 to 20 genes, alternatively between 5 to 15 genes, or alternatively 10 to 20 genes listed in Tables 1, 2, 3, and 4. In some embodiments, the determining step of the methods described herein comprises determining the expression of one or more genes (biomarkers) selected from the group consisting of ALKBH2, AOAH, BICC1, C10orf54, C1RL, C20orf112, C8orf4, CCDC88C, CCNB1, CD84, CD97, CDC20, CDC6, CEBPB, CEBPD, CFB, CHRDL1, CILP, CIRH1A, CLU, CPSF3, CPVL, CSF1R, CTSB, CYP1B1, DDR2, DLGAP5, DUSP1, EMR2, EPB41L3, FBXO32, FOSL2, GAS2L1, GBP3, GINS4, GNAQ, GPR155, GPRIN3, HJURP, IFI44, IFITM2, KCNMB1, LGALS3BP, LRP11, MAFB, MEGF6, MEIS1, MYO1F, MYOF, PCSK5, PDGFC, PHACTR2, PLAT, PRNP, PTPRM, RAB31, RALGDS, RBBP8, RUVBL1, SASH1, SEMA3C, SERPING1, SMOC2, SNRPD1, SPC25, SSH1, TBC1D2B, THEMIS2, TMEM173, TP53INP2, TPSAB1, TRIP13, TXNIP, ULK1, WDR12, WWTR1, XAF1, and ZNF215. In one embodiment, the biomarker is ALKBH2. In another embodiment, the biomarker is AOAH. In yet another embodiment, the biomarker is BICC1. In still another embodiment, the biomarker is C10orf54. In one embodiment, the biomarker is C1RL. In another embodiment, the biomarker is C20orf112. In yet another embodiment, the biomarker is C8orf4. In still another embodiment, the biomarker is CCDC88C. In one embodiment, the biomarker is CCNB1. In another embodiment, the biomarker is CD84. In yet another embodiment, the biomarker is CD97. In still another embodiment, the biomarker is CDC20. In one embodiment, the biomarker is CDC6. In another embodiment, the biomarker is CEBPB. In yet another embodiment, the biomarker is CEBPD. In still another embodiment, the biomarker is CFB. In one embodiment, the biomarker is CHRDL1. In another embodiment, the biomarker is CILP. In yet another embodiment, the biomarker is CIRH1A. In still another embodiment, the biomarker is CLU. In one embodiment, the biomarker is CPSF3. In another embodiment, the biomarker is CPVL. In yet another embodiment, the biomarker is CSF1R. In still another embodiment, the biomarker is CTSB. In one embodiment, the biomarker is CYP1B1. In another embodiment, the biomarker is DDR2. In yet another embodiment, the biomarker is DLGAP5. In still another embodiment, the biomarker is DUSP1. In one embodiment, the biomarker is EMR2. In another embodiment, the biomarker is EPB41L3. In yet another embodiment, the biomarker is FBXO32. In still another embodiment, the biomarker is FOSL2. In one embodiment, the biomarker is GAS2L1. In another embodiment, the biomarker is GBP3. In yet another embodiment, the biomarker is GINS4. In still another embodiment, the biomarker is GNAQ. In one embodiment, the biomarker is GPR155. In another embodiment, the biomarker is GPRIN3. In yet another embodiment, the biomarker is HJURP. In still another embodiment, the biomarker is IFI44. In one embodiment, the biomarker is IFITM2. In another embodiment, the biomarker is KCNMB1. In yet another embodiment, the biomarker is LGALS3BP. In still another embodiment, the biomarker is LRP11. In one embodiment, the biomarker is MAFB. In another embodiment, the biomarker is MEGF6. In yet another embodiment, the biomarker is MEIS1. In still another embodiment, the biomarker is MYO1F. In one embodiment, the biomarker is MYOF. In another embodiment, the biomarker is PCSK5. In yet another embodiment, the biomarker is PDGFC. In still another embodiment, the biomarker is PHACTR2. In one embodiment, the biomarker is PLAT. In another embodiment, the biomarker is PRNP. In yet another embodiment, the biomarker is PTPRM. In still another embodiment, the biomarker is RAB31. In one embodiment, the biomarker is RALGDS. In another embodiment, the biomarker is RBBP8. In yet another embodiment, the biomarker is RUVBL1. In still another embodiment, the biomarker is SASH1. In one embodiment, the biomarker is SEMA3C. In another embodiment, the biomarker is SERPING1. In yet another embodiment, the biomarker is SMOC2. In still another embodiment, the biomarker is SNRPD1. In one embodiment, the biomarker is SPC25. In another embodiment, the biomarker is SSH1. In yet another embodiment, the biomarker is TBC1D2B. In still another embodiment, the biomarker is THEMIS2. In one embodiment, the biomarker is TMEM173. In another embodiment, the biomarker is TP53INP2. In yet another embodiment, the biomarker is TPSAB1. In still another embodiment, the biomarker is TRIP13. In one embodiment, the biomarker is TXNIP. In another embodiment, the biomarker is ULK1. In yet another embodiment, the biomarker is WDR12. In still another embodiment, the biomarker is WWTR1. In one embodiment, the biomarker is XAF1. In another embodiment, the biomarker is ZNF215.

Tables 1, 2, 3, and 4 provide lists of genes that can be used as biomarkers to predict the responsiveness of a patient having a hematological cancer (e.g., DLBCL) to a drug. In certain embodiments, a combination of 1, 2, 3, 4, 5, 6, 7, 8, 9, 10, 11, 12, 13, 14, 15, 20, 25, 30, 35, 40, 45, or more genes listed in Table 1, Table 2, Table 3, or Table 4, or any combination thereof, can be used as biomarkers to predict the responsiveness of a DLBCL patient to a drug. In some embodiments, 1 to 5, 5 to 10, 10 to 15, 15 to 20, 20 to 25, 25 to 30, 30 to 35, 35 to 40, or 40 to 45 genes in Table 1, Table 2, Table 3, or Table 4 can be sued as biomarkers to predict the responsiveness of a DLBCL patient to a drug.

In certain embodiments, a combination of 1, 2, 3, 4, 5, 6, 7, 8, 9, 10, 11, 12, 13, 14, 15, 16, 20, 25, 30, 35, 40, 45, or more genes listed in Table 1; 1, 2, 3, 4, 5, 6, 7, 8, 9, 10, 11, 12, 13, 14, 15, 16, 20, 25, 30, 35, 40, 45, or more genes listed in Table 2; 1, 2, 3, 4, 5, 6, 7, 8, 9, 10, 11, 12, 13, 14, 15, 16, 19, 20, or more genes listed in Table 3; and 1, 2, 3, 4, 5, 6, 7, 8, 9, 10, 11, 12, 13, 14, 15, 16, 19, 20, or more genes listed in Table 4 can be used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 1; 1 to 5, 5 to 10, 10 to 15, 15 to 20, 20 to 25, 25 to 30, 30 to 35, 35 to 40, 40 to 45 genes in Table 2; 1 to 5, 5 to 10, 10 to 15, 15 to 20 genes in Table 3; and 1 to 5, 5 to 10, 10 to 15, 15 to 20 genes in Table 4 are used as biomarkers to predict the responsiveness of a DLBCL patient to a drug.

In some embodiments, a combination of 1, 2, 3, 4, 5, 6, 7, 8, 9, 10, 11, 12, 13, 14, 15, 16, 20, 25, 30, 35, 40, 45, or more genes listed in Table 1 and 1, 2, 3, 4, 5, 6, 7, 8, 9, 10, 11, 12, 13, 14, 15, 16, 20, 25, 30, 35, 40, 45, or more genes listed in Table 2 are used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 1 and 1 to 5, 5 to 10, 10 to 15, 15 to 20, 20 to 25, 25 to 30, 30 to 35, 35 to 40, 40 to 45 genes in Table 2 are used as biomarkers to predict the responsiveness of a DLBCL patient to a drug.

In other embodiments, a combination of 1, 2, 3, 4, 5, 6, 7, 8, 9, 10, 11, 12, 13, 14, 15, 16, 20, 25, 30, 35, 40, 45, or more genes listed in Table 1 and 1, 2, 3, 4, 5, 6, 7, 8, 9, 10, 11, 12, 13, 14, 15, 16, 19, 20, or more genes listed in Table 3 can be used as biomarkers to predict the responsiveness of a DLBCL patient to a drug. In other embodiments, a combination of 1 to 5, 5 to 10, 10 to 15, 15 to 20, 20 to 25, 25 to 30, 30 to 35, 35 to 40, 40 to 45 genes in Table 1 and 1 to 5, 5 to 10, 10 to 15, 15 to 20 genes in Table 3 are used as biomarkers to predict the responsiveness of a DLBCL patient to a drug.

In other embodiments, a combination of 1, 2, 3, 4, 5, 6, 7, 8, 9, 10, 11, 12, 13, 14, 15, 16, 20, 25, 30, 35, 40, 45, or more genes listed in Table 1 and 1, 2, 3, 4, 5, 6, 7, 8, 9, 10, 11, 12, 13, 14, 15, 16, 19, 20, or more genes listed in Table 4 can be used as biomarkers to predict the responsiveness of a DLBCL patient to a drug. In other embodiments, a combination of 1 to 5, 5 to 10, 10 to 15, 15 to 20, 20 to 25, 25 to 30, 30 to 35, 35 to 40, 40 to 45 genes in Table 1 and 1 to 5, 5 to 10, 10 to 15, 15 to 20 genes in Table 4 are used as biomarkers to predict the responsiveness of a DLBCL patient to a drug.

In other embodiments, a combination of 1, 2, 3, 4, 5, 6, 7, 8, 9, 10, 11, 12, 13, 14, 15, 16, 20, 25, 30, 35, 40, 45, or more genes listed in Table 2 and 1, 2, 3, 4, 5, 6, 7, 8, 9, 10, 11, 12, 13, 14, 15, 16, 19, 20, or more genes listed in Table 3 can be used as biomarkers to predict the responsiveness of a DLBCL patient to a drug. In other embodiments, a combination of 1 to 5, 5 to 10, 10 to 15, 15 to 20, 20 to 25, 25 to 30, 30 to 35, 35 to 40, 40 to 45 genes in Table 2 and 1 to 5, 5 to 10, 10 to 15, 15 to 20 genes in Table 3 are used as biomarkers to predict the responsiveness of a DLBCL patient to a drug.

In other embodiments, a combination of 1, 2, 3, 4, 5, 6, 7, 8, 9, 10, 11, 12, 13, 14, 15, 16, 20, 25, 30, 35, 40, 45, or more genes listed in Table 2 and 1, 2, 3, 4, 5, 6, 7, 8, 9, 10, 11, 12, 13, 14, 15, 16, 19, 20, or more genes listed in Table 4 can be used as biomarkers to predict the responsiveness of a DLBCL patient to a drug. In other embodiments, a combination of 1 to 5, 5 to 10, 10 to 15, 15 to 20, 20 to 25, 25 to 30, 30 to 35, 35 to 40, 40 to 45 genes in Table 2 and 1 to 5, 5 to 10, 10 to 15, 15 to 20 genes in Table 4 are used as biomarkers to predict the responsiveness of a DLBCL patient to a drug.

In other embodiments, a combination of 1, 2, 3, 4, 5, 6, 7, 8, 9, 10, 11, 12, 13, 14, 15, 16, 20, or more genes listed in Table 3 and 1, 2, 3, 4, 5, 6, 7, 8, 9, 10, 11, 12, 13, 14, 15, 16, 19, 20, or more genes listed in Table 4 can be used as biomarkers to predict the responsiveness of a DLBCL patient to a drug. In other embodiments, a combination of 1 to 5, 5 to 10, 10 to 15, 15 to 20, 20 to 25 genes in Table 3 and 1 to 5, 5 to 10, 10 to 15, 15 to 20 genes in Table 4 are used as biomarkers to predict the responsiveness of a DLBCL patient to a drug.

In some embodiments, a combination of 1, 2, 3, 4, 5, 6, 7, 8, 9, 10, 11, 12, 13, 14, 15, 16, 20, 25, 30, 35, 40, 45, or more genes listed in Table 1, and 1, 2, 3, 4, 5, 6, 7, 8, 9, 10, 11, 12, 13, 14, 15, 16, 20, 25, 30, 35, 40, 45, or more genes listed in Table 2, and 1, 2, 3, 4, 5, 6, 7, 8, 9, 10, 11, 12, 13, 14, 15, 16, 20, or more genes listed in Table 3 are used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 1, and 1 to 5, 5 to 10, 10 to 15, 15 to 20, 20 to 25, 25 to 30, 30 to 35, 35 to 40, 40 to 45 genes in Table 2, and 1 to 5, 5 to 10, 10 to 15, 15 to 20, 20 to 25 genes in Table 3 are used as biomarkers to predict the responsiveness of a DLBCL patient to a drug.

In some embodiments, a combination of 1, 2, 3, 4, 5, 6, 7, 8, 9, 10, 11, 12, 13, 14, 15, 16, 20, 25, 30, 35, 40, 45, or more genes listed in Table 1, and 1, 2, 3, 4, 5, 6, 7, 8, 9, 10, 11, 12, 13, 14, 15, 16, 20, 25, 30, 35, 40, 45, or more genes listed in Table 2, and 1, 2, 3, 4, 5, 6, 7, 8, 9, 10, 11, 12, 13, 14, 15, 16, 20, or more genes listed in Table 4 are used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 1, and 1 to 5, 5 to 10, 10 to 15, 15 to 20, 20 to 25, 25 to 30, 30 to 35, 35 to 40, 40 to 45 genes in Table 2, and 1 to 5, 5 to 10, 10 to 15, 15 to 20 genes in Table 4 are used as biomarkers to predict the responsiveness of a DLBCL patient to a drug.

In some embodiments, a combination of 1, 2, 3, 4, 5, 6, 7, 8, 9, 10, 11, 12, 13, 14, 15, 16, 20, 25, 30, 35, 40, 45, or more genes listed in Table 2, and 1, 2, 3, 4, 5, 6, 7, 8, 9, 10, 11, 12, 13, 14, 15, 16, 20, or more genes listed in Table 3, and 1, 2, 3, 4, 5, 6, 7, 8, 9, 10, 11, 12, 13, 14, 15, 16, 20, or more genes listed in Table 4 are used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 2, and 1 to 5, 5 to 10, 10 to 15, 15 to 20, 20 to 25 genes in Table 3, and 1 to 5, 5 to 10, 10 to 15, 15 to 20 genes in Table 4 are used as biomarkers to predict the responsiveness of a DLBCL patient to a drug.

In some embodiments, a combination of 1, 2, 3, 4, 5, 6, 7, 8, 9, 10, 11, 12, 13, 14, 15, 16, 20, 25, 30, 35, 40, 45, or more genes listed in Table 1, and 1, 2, 3, 4, 5, 6, 7, 8, 9, 10, 11, 12, 13, 14, 15, 16, 20, or more genes listed in Table 3, and 1, 2, 3, 4, 5, 6, 7, 8, 9, 10, 11, 12, 13, 14, 15, 16, 20, or more genes listed in Table 4 are used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 1, and 1 to 5, 5 to 10, 10 to 15, 15 to 20, 20 to 25 genes in Table 3, and 1 to 5, 5 to 10, 10 to 15, 15 to 20 genes in Table 4 are used as biomarkers to predict the responsiveness of a DLBCL patient to a drug.

In some embodiments, a combination of 1, 2, 3, 4, 5, 6, 7, 8, 9, 10, 11, 12, 13, 14, 15, 16, 20, 25, 30, 35, 40, 45, or more genes listed in Table 1, and 1, 2, 3, 4, 5, 6, 7, 8, 9, 10, 11, 12, 13, 14, 15, 16, 20, 25, 30, 35, 40, 45, or more genes listed in Table 2, and 1, 2, 3, 4, 5, 6, 7, 8, 9, 10, 11, 12, 13, 14, 15, 16, 20, or more genes listed in Table 3, and 1, 2, 3, 4, 5, 6, 7, 8, 9, 10, 11, 12, 13, 14, 15, 16, 20, or more genes listed in Table 4 are used as biomarkers to predict the responsiveness of a DLBCL patient to a drug. In some embodiments, a combination of 1 to 5, 5 to 10, 10 to 15, 15 to 20, 20 to 25, 25 to 30, 30 to 35, 35 to 40, 40 to 45 genes in Table 1, and 1 to 5, 5 to 10, 10 to 15, 15 to 20, 20 to 25, 25 to 30, 30 to 35, 35 to 40, 40 to 45 genes in Table 2, and 1 to 5, 5 to 10, 10 to 15, 15 to 20, 20 to 25 genes in Table 3, and 1 to 5, 5 to 10, 10 to 15, 15 to 20 genes in Table 4 are used as biomarkers to predict the responsiveness of a DLBCL patient to a drug.

Additionally, in some embodiments, all of the genes listed in Table 1, Table 2, Table 3, and Table 4 can be used as biomarkers to predict the responsiveness of a DLBCL patient to a drug.

The biomarkers in Table 1, Table 2, Table 3, and/or Table 4 can be used by a health professional in combination with other factors to determine whether or not to treat the DLBCL patient with the drug, e.g., lenalidomide or Compound A.

In certain embodiments, in accordance with the methods described herein, the gene expression profile or data is derived from the same type of biological sample. In other words, the biological sample used to generate each gene expression profile or data referenced in the methods is the same type of biological sample. In some embodiments, the biological samples are tumor biopsy samples.

In some embodiments, the determining step of the methods described herein comprises detecting the presence and/or amount of a complex in the biological sample, wherein the presence and/or amount of the complex indicates the expression level of the genes. The complex detected in the methods described herein can be a hybridization complex and in some embodiments, the hybridization complex is attached to a solid support. In further embodiments, the complex is detectably labeled.

In some embodiments, the determining step of the methods described herein comprises detecting the presence and/or amount of a reaction product in the biological sample, wherein the presence and/or amount of the reaction product indicates the expression level of the genes in each subset of genes. In further embodiments, the reaction product is detectably labeled.

In some embodiments, the expression of the genes or biomarkers provided herein is determined by determining the protein levels of the genes or biomarkers. In other embodiments, the expression of the genes or biomarkers provided herein is determined by determining the mRNA levels of the genes or biomarkers. In yet other embodiments, the expression of the genes provided herein is determined by determining the levels of cDNA generated using the mRNA of a gene or biomarker. Accordingly, the upregulation or downregulation of the nucleic acids (e.g., mRNA or cDNA) or proteins of the genes provided herein (e.g., those listed in Tables 1-4) can be used to predict responsiveness of a patient having a hematological cancer (e.g., DLBCL) to a compound treatment.

In some embodiments, a statistical analysis or other analysis is performed on data from the assay utilized to measure an RNA transcript or protein. In certain specific embodiments, the p value of those RNA transcripts or proteins differentially expressed is 0.1, 0.5, 0.4, 0.3, 0.2, 0.01, 0.05, 0.001, or 0.0001. In some embodiments, the p-value provided herein is the output of a statistical test of difference between two or more groups of values or data examples. In specific embodiments, a false discovery rate (FDR) of 10%, 9%, 8%, 7%, 6%, 5%, 4%, 3%, 2% or less is selected. FDR correction and associated thresholds are commonly applied to correct p-values for multiple hypothesis testing, i.e. applying the same test or comparison to many groups of values before seeking to assign statistical significance to a subset of the differences observed. In some embodiments, hypothesis testing for difference may also be applied to output of a functional transformation of assay output. In some embodiments, additional processes other than tests of difference can be used to classify or predict drug response from assay output.

In certain embodiments, the hematological cancer is selected from the group consisting of leukemia, lymphoma, and multiple myeloma. In one embodiment, the hematological cancer is leukemia. In another embodiment, the hematological cancer is lymphoma. In still another embodiment, the hematological cancer is multiple myeloma.

In some embodiments, the lymphoma is DLBCL. In one embodiment, the DLBCL is refractory in the first patient. In another embodiment, the DLBCL is relapsed in the first patient. In yet another embodiment, the DLBCL is a GCB-DLBCL in the first patient. In still another embodiment, the DLBCL is ABC-DLBCL in the first patient. In yet another embodiment, the DLBCL is NOS-DLBCL in the first patient.

In some embodiments, the methods described herein include determining the gene expression profile of a subset of genes in DLBCL patients that have taken the drug prior to treatment with the drug, wherein each subset of genes relates to a tumor biopsy composition.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of B cells in the first tumor sample, and (c) comparing the proportion of B cells in the first tumor sample with the proportion of B cells in a second tumor sample from a second patient having the same type of hematological cancer, wherein the second patient's hematological cancer is clinically insensitive to treatment with an immunomodulatory therapy, and wherein a lower proportion of B cells in the first tumor sample is measured relative the proportion of B cells in the second tumor sample indicates that the hematological cancer in the first patient will be clinically sensitive to treatment with the immunomodulatory therapy.

In certain embodiments, the B cells are CD20+ B cells.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of dendritic cells in the first tumor sample, and (c) comparing the proportion of dendritic cells in the first tumor sample with the proportion of dendritic cells in a second tumor sample from a second patient having the same type of hematological cancer, wherein the second patient's hematological cancer is clinically insensitive to treatment with an immunomodulatory therapy, and wherein a higher proportion of dendritic cells in the first tumor sample is measured relative the proportion of dendritic cells in the second tumor sample indicates that the hematological cancer in the first patient will be clinically sensitive to treatment with the immunomodulatory therapy.

In certain embodiments, the dendritic cells are CD11c+ dendritic cells.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of T cells in the first tumor sample, and (c) comparing the proportion of T cells in the first tumor sample with the proportion of T cells in a second tumor sample from a second patient having the same type of hematological cancer, wherein the second patient's hematological cancer is clinically insensitive to treatment with an immunomodulatory therapy, and wherein a higher proportion of T cells in the first tumor sample is measured relative the proportion of T cells in the second tumor sample indicates that the hematological cancer in the first patient will be clinically sensitive to treatment with the immunomodulatory therapy.

In certain embodiments, the T cells are CD3+ T cells. In certain embodiments, the T cells are CD8+ T cells.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of macrophage cells in the first tumor sample, and (c) comparing the proportion of macrophage cells in the first tumor sample with the proportion of macrophage cells in a second tumor sample from a second patient having the same type of hematological cancer, wherein the second patient's hematological cancer is clinically insensitive to treatment with an immunomodulatory therapy, and wherein a higher proportion of macrophage cells in the first tumor sample is measured relative the proportion of macrophage cells in the second tumor sample indicates that the hematological cancer in the first patient will be clinically sensitive to treatment with the immunomodulatory therapy.

In certain embodiments, the macrophage cells are CD163+ macrophage cells.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of PD-L1+ cells in the first tumor sample, and (c) comparing the proportion of PD-L1+ cells in the first tumor sample with the proportion of PD-L1+ cells in a second tumor sample from a second patient having the same type of hematological cancer, wherein the second patient's hematological cancer is clinically insensitive to treatment with an immunomodulatory therapy, and wherein a lower proportion of PD-L1+ cells in the first tumor sample is measured relative the proportion of PD-L1+ cells in the second tumor sample indicates that the hematological cancer in the first patient will be clinically sensitive to treatment with the immunomodulatory therapy.

In another aspect, provided herein are for predicting the clinical sensitivity of a hematological cancer to treatment with an immunomodulatory therapy comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and wherein that the proportion of the immune cells in the first tumor sample is (i) similar to the proportion of the same immune cells in tumor samples from patients having the same type of hematological cancer which are clinically sensitive to the immunomodulatory therapy, and (ii) not similar to the proportion of the same immune cells in tumor samples from patients having the same type of hematological cancer which are clinically insensitive to the immunomodulatory therapy indicates that the hematological cancer in the first page will be clinically sensitive to treatment with the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In another aspect, provided herein are methods for managing or treating a hematological cancer comprising: (a) obtaining a first tumor sample from a first patient having the hematological cancer, (b) measuring the proportion of B cells in the first tumor sample, (c) comparing the proportion of B cells in the first tumor sample with the proportion of B cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a lower proportion of B cells in the first tumor sample is measured relative the proportion of B cells in the second tumor sample.

In certain embodiments, the B cells are CD20+ B cells.

In another aspect, provided herein are methods for managing or treating a hematological cancer comprising: (a) obtaining a first tumor sample from a first patient having the hematological cancer, (b) measuring the proportion of dendritic cells in the first tumor sample, (c) comparing the proportion of dendritic cells in the first tumor sample with the proportion of dendritic cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of dendritic cells in the first tumor sample is measured relative the proportion of dendritic cells in the second tumor sample.

In certain embodiments, the dendritic cells are CD11c+ dendritic cells.

In another aspect, provided herein are methods for managing or treating a hematological cancer comprising: (a) obtaining a first tumor sample from a first patient having the hematological cancer, (b) measuring the proportion of T cells in the first tumor sample, (c) comparing the proportion of T cells in the first tumor sample with the proportion of T cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of T cells in the first tumor sample is measured relative the proportion of T cells in the second tumor sample.

In certain embodiments, the T cells are CD3+ T cells. In certain embodiments, the T cells are CD8+ T cells.

In another aspect, provided herein are methods for managing or treating a hematological cancer comprising: (a) obtaining a first tumor sample from a first patient having the hematological cancer, (b) measuring the proportion of macrophage cells in the first tumor sample, (c) comparing the proportion of macrophage cells in the first tumor sample with the proportion of macrophage cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of macrophage cells in the first tumor sample is measured relative the proportion of macrophage cells in the second tumor sample.

In certain embodiments, the macrophage cells are CD163+ macrophage cells.

In another aspect, provided herein are methods for managing or treating a hematological cancer comprising: (a) obtaining a first tumor sample from a first patient having the hematological cancer, (b) measuring the proportion of PD-L1+ cells in the first tumor sample, (c) comparing the proportion of PD-L1+ cells in the first tumor sample with the proportion of PD-L1+ cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of PD-L1+ cells in the first tumor sample is measured relative the proportion of PD-L1+ cells in the second tumor sample.

In another aspect, provided herein are for managing or treating a hematological cancer comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and (d) administering the immunomodulatory therapy to the first patient if the proportion of the immune cells in the first tumor sample is (i) similar to the proportion of the same immune cells in tumor samples from patients having the same type of hematological cancer which are clinically sensitive to the immunomodulatory therapy, and (ii) not similar to the proportion of the same immune cells in tumor samples from patients having the same type of hematological cancer which are clinically insensitive to the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In certain embodiments of the foregoing paragraphs in this section, the second patient is a single patient. In other embodiments of the foregoing paragraphs in this section, the second patient is a population of patients. In specific embodiments, the population comprises 5, 10, 15, 20, 25, 30, 35, 40, 45, 50, 55, 60, 65, 70, 75, 80, 85, 90, 95, 100, 125, 150, 200, 225, 250, 300 or more patients.

Techniques known to one skilled in the art may be used to measure the proportion of cells in a tumor sample. In certain embodiments, the proportion of cells is measured by flow cytometry, immunofluorscence, enzyme-linked immunosorbent assay-based methodologies (ELISA) and similar assays known in the art. See Section 5.8, infra, regarding techniques for measuring and distinguishing cell types. In other embodiments, the proportion of cells is measured by inference from gene expression profiles.

In accordance with the methods described herein, the hematological cancer can be any hematological cancer. In a specific embodiment, the hematological cancer is a lymphoma. In another specific embodiment, the hematological cancer is a non-Hodgkin's lymphoma. In yet another embodiment, the hematological cancer is a diffuse target B-cell lymphoma (DLBCL). In certain embodiments, the DLBCL is a germinal center B-cell-like DLBCL. In other embodiments, the DLCBL is an activated B-cell-like DLBCL.

In accordance with the methods described herein, the immunomodulatory therapy can be any therapy that modulates the immune system or immune response. In a specific embodiment, the immunomodulatory therapy is lenolidomide. In a specific embodiment, the immunomodulatory therapy is Compound A.

### 5.3 Treatment Compound for Use in Methods of Treating DLBCL

The treatment compounds provided herein can be used in all methods of treatment of a hematological cancer in a patient as provided herein.

In one aspect, provided herein are methods for treating a DLBCL patient with lenalidomide or Compound A, wherein the method uses the biomarkers and/or classifiers identified in Tables 1, 2, 3 and/or 4. In another aspect, provided herein are methods for treating a patient having a hematological cancer with a treatment compound.

In some embodiments, provided herein is a method for treating a patient having a hematological cancer with a treatment compound, comprising:

(a) obtaining a biological sample from the patient having the hematological cancer;

(b) determining the expression level of one, two, three, four, five, or more of the genes identified in Tables 1, 2, 3, and/or 4;

(c) comparing the expression level of the one, two, three, four, five, or more of the genes identified in Tables 1, 2, 3, and/or 4 in the biological sample with a reference expression level of the same genes; and

(d) administering the treatment compound to the patient if the one, two, three, four, five, or more of the genes in the biological sample are differentially expressed relative to the reference expression level of the same genes;

wherein the differential expression level of the one, two, three, four, five, or more of the genes in the biological sample relative to the reference expression level of the one, two, three, four, five, or more of the genes indicates that the first patient will be responsive to the treatment compound; and

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In certain embodiments, the reference expression level of the one, two, three, four, five, or more of the genes identified in Tables 1, 2, 3, and/or 4 is the expression level of the same genes in a reference sample. In one embodiment, the reference sample is a second biological sample. In another embodiment, the reference sample is synthetically derived.

In certain embodiments, provided herein is a method for treating a patient having a hematological cancer with a treatment compound, comprising:

(a) obtaining a first biological sample from a first patient having the hematological cancer;

(b) determining the expression level of one, two, three, four, five, or more of the genes identified in Tables 1, 2, 3, and/or 4;

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is not responsive to the treatment compound; and

(d) administering the treatment compound to the first patient if the one, two, three, four, five, or more of the genes in the first biological sample are differentially expressed relative to the expression level of the one, two, three, four, five, or more of the genes in the second biological sample,

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, provided herein is a method for treating a patient having a hematological cancer with a treatment compound, comprising:

(a) obtaining a first biological sample from a first patient having the hematological cancer;

(b) determining the expression level of one, two, three, four, five, or more of the genes identified in Tables 1, 2, 3, and/or 4;

(c) comparing the expression level of the one, two, three, four, five, or more of the genes identified in Tables 1, 2, 3, and/or 4 in the first biological sample with the expression level of the same genes in a second biological sample obtained from a second patient having the hematological cancer, wherein the second patient is responsive to the treatment compound; and

(d) administering the treatment compound to the first patient if the one, two, three, four, five, or more of the genes in the first biological sample are similarly expressed relative to the expression level of the one, two, three, four, five, or more of the genes in the second biological sample,

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In other embodiments, provided herein is a method for treating a patient having a hematological cancer with a treatment compound, comprising:

(a) obtaining a first biological sample from a first patient having the hematological cancer;

(b) determining the expression level of one, two, three, four, five, or more of the genes identified in Tables 1, 2, 3, and/or 4;

(c) comparing the gene expression profile of the genes or subset of genes in the first biological sample to
(i) the gene expression profile of the one, two, three, four, five, or more of the genes in a second biological sample from a second patient having the hematological cancer, wherein the second patient is responsive to the treatment compound, and
(ii) the gene expression profile of the one, two, three, four, five, or more of the genes in a third biological sample from a third patient having the hematological cancer, wherein the third patient is not responsive to the treatment compound; and

(d) administering the treatment compound to the first patient if: (i) the one, two, three, four, five, or more of the genes in the first biological sample are similarly expressed relative to the expression level of the one, two, three, four, five, or more of the genes in the second biological sample, and/or (ii) the one, two, three, four, five, or more of the genes in the first biological sample are differentially expressed relative to the expression level of the one, two, three, four, five, or more of the genes in the third biological sample,

wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

In some embodiments, the one, two, three, four, five, or more of genes are a subset of the genes in Table 1, a subset of the genes in Table 2, a subset of the genes in Table 3, and/or a subset of the genes in Table 4. In other embodiments, the one, two, three, four, five, or more of genes are a combination of a subset of the genes in two tables selected from Tables 1-4. In yet other embodiments, the one, two, three, four, five, or more of genes are a combination of a subset of the genes in three tables selected from Tables 1-4. In still other embodiments, the one, two, three, four, five, or more of genes are a combination of a subset of the genes in Table 1, a subset of the genes in Table 2, a subset of the genes in Table 3, and a subset of the genes in Table 4.

In some embodiments, expression levels of multiple genes (biomarkers) provided herein are used to predict a patient's response to the compound provided herein. When expression levels of multiple genes (biomarkers) are used to predict a patient's response to a treatment with the compound provided herein, e.g., lenalidomide or Compound A, any classifier that classifies based on two or more features can be used in the present disclosure. For example, in some embodiments, a population of DLBCL patients that have received a treatment with the compound provide herein (e.g., lenalidomide or Compound A) are divided into two groups based on their responsiveness to the compound treatment, i.e., responsive patient group and non-responsive patient group. The expression levels of two or more genes provided herein (e.g., those listed in Tables 1-4) for each patient in the population are analyzed using a classifier and a score can be generated. A reference score or a threshold score can be generated based on the scores of the responsive patients and the scores of non-responsive patients. Such reference score can be used to predict a patient's responsiveness to the compound based on the expression levels of the genes of this patient.

In some embodiments, the methods provided herein further comprise (a) generating a score of the sample based on the expression levels of the genes or a subset thereof provided herein in the sample; and (b) determining the probability of the subject being responsive to the compound provided herein by comparing the score of the sample to a reference score.

In some embodiments, the methods provided herein further comprise: (a) determining the expression levels of the genes or a subset thereof provided herein in a biological sample from a population of subjects that previously have been administered with the compound provided herein, (b) generating a score for the expression levels of the genes or a subset thereof provided herein for each subject of the population; (c) differentiating the subjects that are responsive to the compound provided herein from those subjects that are not responsive to the compound provided herein; and (d) determining responsiveness to the compound provided herein based on the scores for the subjects that are responsive to the compound and those subjects that are not responsive to the compound.

In other embodiments, the methods provided herein further comprise: (a) determining the expression levels of the genes or a subset thereof provided herein in a biological sample from a population of subjects that previously have been administered with the compound, (b) generating a score for the expression levels of the genes or a subset thereof provided herein for each subject of the population; (c) differentiating the subjects that are responsive to the compound provided herein from those subjects that are not responsive to compound provided herein; and (d) generating a reference score that is predictive of the responsiveness of a subject to the compound provided herein using a model based on the scores for the subjects that are responsive to the compound and those subjects that are not responsive to the compound.

In another aspect, provided herein are methods for treating a patient having a hematological cancer with a treatment compound, comprising: (a) obtaining a biological sample from a patient having the hematological cancer; (b) determining the expression of the genes or a certain subset of genes set forth in Tables 1, 2, 3, and/or 4, or any combination thereof, in the biological sample and generating a score based on the expression of the genes or subset of genes, (c) comparing the score to a reference score; and (d) administering the treatment compound to the patient if the score is similar to the reference score. In specific embodiments, the reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In another aspect, provided herein are methods for treating a patient having a hematological cancer with a treatment compound, comprising: (a) obtaining a biological sample from a patient having the hematological cancer; (b) determining the expression of the genes or a certain subset of genes set forth in Tables 1, 2, 3, and/or 4, or any combination thereof, in the biological sample and generating a score based on the expression of the genes or subset of genes, (c) comparing the score to a first reference score and a second reference score; and (d) administering the treatment compound to the patient if (i) the score is similar to the first reference score, and/or (ii) the score is different from the second reference score. In specific embodiments, the first reference score is based on the expression of the same genes or subset of genes in a population of patients having the hematological cancer who are responsive to the treatment compound, and the second reference score is based on the expression of the same genes or subset of genes in a population of patients having the hematological cancer who are responsive to the treatment compound. In a specific embodiment, the treatment compound is a compound disclosed herein. In one embodiment, the treatment compound is lenalidomide. In another embodiment, the treatment compound is Compound A.

In certain embodiments, the hematological cancer is selected from the group consisting of leukemia, lymphoma, and multiple myeloma. In one embodiment, the hematological cancer is leukemia. In another embodiment, the hematological cancer is lymphoma. In still another embodiment, the hematological cancer is multiple myeloma.

In some embodiments, the lymphoma is DLBCL. In one embodiment, the DLBCL is refractory in the first patient. In another embodiment, the DLBCL is relapsed in the first patient. In yet another embodiment, the DLBCL is a GCB-DLBCL in the first patient. In still another embodiment, the DLBCL is ABC-DLBCL in the first patient. In yet another embodiment, the DLBCL is NOS-DLBCL in the first patient.

In some embodiments, the determining step of the methods described herein comprising determining the expression of the genes or a certain subset of genes set forth in Table 1 in the first biological sample. For example, in certain embodiments, the subset of genes can include 2, 3, 4, 5, 6, 7, 8, 9, 10, 11, 12, 13, 14, 15, 16, 20, 25, 30, 35, 40, 45, or more genes listed in Table 1. In another embodiment, the subset of genes can include between 2 to 40 genes, alternatively between 3 to 45 genes, alternatively between 5 to 15 genes, or alternatively between 10 to 20 genes listed in Table 1. In some embodiments, the determining step of the methods described herein comprises determining the expression of one or more genes selected from the group consisting of BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, and ZNF215.

In some embodiments, the determining step of the methods described herein comprising determining the expression of the genes or a certain subset of genes set forth in Table 2 in the first biological sample. For example, in certain embodiments, the subset of genes can include 2, 3, 4, 5, 6, 7, 8, 9, 10, 11, 12, 13, 14, 15, 16, 20, 25, 30, 35, 40, 45, or more genes listed in Table 2. In another embodiment, the subset of genes can include between 2 to 40 genes, or alternatively between 3 to 45 genes, alternatively between 5 to 15 genes, or alternatively 10 to 20 genes listed in Table 2. In some embodiments, the determining step of the methods described herein comprises determining the expression of one or more genes selected from the group consisting of ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, and ZNF215.

In some embodiments, the determining step of the methods described herein comprising determining the expression of the genes or a certain subset of genes set forth in Table 3 in the first biological sample. For example, in certain embodiments, the subset of genes can include 2, 3, 4, 5, 6, 7, 8, 9, 10, 11, 12, 13, 14, 15, 16, 20, or more genes listed in Table 3. In another embodiment, the subset of genes can include between 2 to 20 genes, or alternatively between 3 to 24 genes, alternatively between 5 to 15 genes, or alternatively 10 to 24 genes listed in Table 3. In some embodiments, the determining step of the methods described herein comprises determining the expression of one or more genes selected from the group consisting of C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, and ZNF215.

In some embodiments, the determining step of the methods described herein comprising determining the expression of the genes or a certain subset of genes set forth in Table 4 in the first biological sample. For example, in certain embodiments, the subset of genes can include 2, 3, 4, 5, 6, 7, 8, 9, 10, 11, 12, 13, 14, 15, 16, 20, or more genes listed in Table 4. In another embodiment, the subset of genes can include between 2 to 20 genes, or alternatively between 3 to 24 genes, alternatively between 5 to 15 genes, or alternatively 10 to 24 genes listed in Table 4. In some embodiments, the determining step of the methods described herein comprises determining the expression of one or more genes selected from the group consisting of C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, and ZNF215.

In some embodiments, the determining step of the methods described herein comprising determining the expression of the genes or a certain subset of genes set forth in a combination of Tables 1, 2, 3, and 4 in the first biological sample. For example, in certain embodiments, the subset of genes can include 2, 3, 4, 5, 6, 7, 8, 9, 10, 11, 12, 13, 14, 15, 16, 20, 25, 30, 35, 40, 50, 60, 70, or more genes listed in Tables 1, 2, 3, and 4. In another embodiment, the subset of genes can include between 2 to 71 genes, alternatively between 2 to 20 genes, alternatively between 3 to 20 genes, alternatively between 5 to 15 genes, or alternatively 10 to 20 genes listed in Tables 1, 2, 3, and 4. In some embodiments, the determining step of the methods described herein comprises determining the expression of one or more genes (biomarkers) selected from the group consisting of ALKBH2, AOAH, BICC1, C10orf54, C1RL, C20orf112, C8orf4, CCDC88C, CCNB1, CD84, CD97, CDC20, CDC6, CEBPB, CEBPD, CFB, CHRDL1, CILP, CIRH1A, CLU, CPSF3, CPVL, CSF1R, CTSB, CYP1B1, DDR2, DLGAP5, DUSP1, EMR2, EPB41L3, FBXO32, FOSL2, GAS2L1, GBP3, GINS4, GNAQ, GPR155, GPRIN3, HJURP, IFI44, IFITM2, KCNMB1, LGALS3BP, LRP11, MAFB, MEGF6, MEIS1, MYO1F, MYOF, PCSK5, PDGFC, PHACTR2, PLAT, PRNP, PTPRM, RAB31, RALGDS, RBBP8, RUVBL1, SASH1, SEMA3C, SERPING1, SMOC2, SNRPD1, SPC25, SSH1, TBC1D2B, THEMIS2, TMEM173, TP53INP2, TPSAB1, TRIP13, TXNIP, ULK1, WDR12, WWTR1, XAF1, and ZNF215. In one embodiment, the biomarker is ALKBH2. In another embodiment, the biomarker is AOAH. In yet another embodiment, the biomarker is BICC1. In still another embodiment, the biomarker is C10orf54. In one embodiment, the biomarker is C1RL. In another embodiment, the biomarker is C20orf112. In yet another embodiment, the biomarker is C8orf4. In still another embodiment, the biomarker is CCDC88C. In one embodiment, the biomarker is CCNB1. In another embodiment, the biomarker is CD84. In yet another embodiment, the biomarker is CD97. In still another embodiment, the biomarker is CDC20. In one embodiment, the biomarker is CDC6. In another embodiment, the biomarker is CEBPB. In yet another embodiment, the biomarker is CEBPD. In still another embodiment, the biomarker is CFB. In one embodiment, the biomarker is CHRDL1. In another embodiment, the biomarker is CILP. In yet another embodiment, the biomarker is CIRH1A. In still another embodiment, the biomarker is CLU. In one embodiment, the biomarker is CPSF3. In another embodiment, the biomarker is CPVL. In yet another embodiment, the biomarker is CSF1R. In still another embodiment, the biomarker is CTSB. In one embodiment, the biomarker is CYP1B1. In another embodiment, the biomarker is DDR2. In yet another embodiment, the biomarker is DLGAP5. In still another embodiment, the biomarker is DUSP1. In one embodiment, the biomarker is EMR2. In another embodiment, the biomarker is EPB41L3. In yet another embodiment, the biomarker is FBXO32. In still another embodiment, the biomarker is FOSL2. In one embodiment, the biomarker is GAS2L1. In another embodiment, the biomarker is GBP3. In yet another embodiment, the biomarker is GINS4. In still another embodiment, the biomarker is GNAQ. In one embodiment, the biomarker is GPR155. In another embodiment, the biomarker is GPRIN3. In yet another embodiment, the biomarker is HJURP. In still another embodiment, the biomarker is IFI44. In one embodiment, the biomarker is IFITM2. In another embodiment, the biomarker is KCNMB1. In yet another embodiment, the biomarker is LGALS3BP. In still another embodiment, the biomarker is LRP11. In one embodiment, the biomarker is MAFB. In another embodiment, the biomarker is MEGF6. In yet another embodiment, the biomarker is MEIS1. In still another embodiment, the biomarker is MYO1F. In one embodiment, the biomarker is MYOF. In another embodiment, the biomarker is PCSK5. In yet another embodiment, the biomarker is PDGFC. In still another embodiment, the biomarker is PHACTR2. In one embodiment, the biomarker is PLAT. In another embodiment, the biomarker is PRNP. In yet another embodiment, the biomarker is PTPRM. In still another embodiment, the biomarker is RAB31. In one embodiment, the biomarker is RALGDS. In another embodiment, the biomarker is RBBP8. In yet another embodiment, the biomarker is RUVBL1. In still another embodiment, the biomarker is SASH1. In one embodiment, the biomarker is SEMA3C. In another embodiment, the biomarker is SERPING1. In yet another embodiment, the biomarker is SMOC2. In still another embodiment, the biomarker is SNRPD1. In one embodiment, the biomarker is SPC25. In another embodiment, the biomarker is SSH1. In yet another embodiment, the biomarker is TBC1D2B. In still another embodiment, the biomarker is THEMIS2. In one embodiment, the biomarker is TMEM173. In another embodiment, the biomarker is TP53INP2. In yet another embodiment, the biomarker is TPSAB1. In still another embodiment, the biomarker is TRIP13. In one embodiment, the biomarker is TXNIP. In another embodiment, the biomarker is ULK1. In yet another embodiment, the biomarker is WDR12. In still another embodiment, the biomarker is WWTR1. In one embodiment, the biomarker is XAF1. In another embodiment, the biomarker is ZNF215.

In some embodiments, the determining step of the methods provided herein comprises determining the expression of all the genes listed in Table 1. In some embodiments, the determining step of the methods provided herein comprises determining the expression of all the genes listed in Table 2. In some embodiments, the determining step of the methods provided herein comprises determining the expression of all the genes listed in Table 3. In some embodiments, the determining step of the methods provided herein comprises determining the expression of all the genes listed in Table 4.

In certain embodiments, in accordance with the methods described herein, the gene expression provide or data is derived from the same type of biological sample. In other words, the biological sample used to generate each gene expression profile or data referenced in the methods is the same type of biological sample. In some embodiments, the biological samples are tumor biopsy samples.

In some embodiments, the determining step of the methods described herein comprise detecting the presence and/or amount of a complex in the biological sample, wherein the presence and/or amount of the complex indicates the expression level of the genes. The complex detected in the methods described herein can be a hybridization complex and in some embodiments, the hybridization complex is attached to a solid support. In further embodiments, the complex is detectably labeled.

In some embodiments, the determining step of the methods described herein comprise detecting the presence and/or amount of a reaction product in the biological sample, wherein the presence and/or amount of the reaction product indicates the expression level of the genes in each subset of genes. In further embodiments, the reaction product is detectably labeled.

In some embodiments, the expression of the genes or biomarkers provided herein is determined by determining the protein levels of the genes or biomarkers. In other embodiments, the expression of the genes or biomarkers provided herein is determined by determining the mRNA levels of the genes or biomarkers. In yet other embodiments, the expression of the genes provided herein is determined by determining the levels of cDNA generated using mRNA of the genes or biomarkers. Accordingly, the upregulation or downregulation of nucleic acids (e.g., mRNA or cDNA) or proteins of the genes provided herein (e.g., those listed in Tables 1-4) can be used to predict responsiveness of a patient having a hematological cancer (e.g., DLBCL) to a compound treatment.

In some embodiments, a statistical analysis or other analysis is performed on data from the assay utilized to measure an RNA transcript or protein. In certain specific embodiments, the p value of those RNA transcripts or proteins differentially expressed is 0.1, 0.5, 0.4, 0.3, 0.2, 0.01, 0.05, 0.001, or 0.0001. In some embodiments, the p-value provided herein is the output of a statistical test of difference between two or more groups of values or data examples. In specific embodiments, a false discovery rate (FDR) of 10%, 9%, 8%, 7%, 6%, 5%, 4%, 3%, 2% or less is selected. FDR correction and associated thresholds are commonly applied to correct p-values for multiple hypothesis testing, i.e. applying the same test or comparison to many groups of values before seeking to assign statistical significance to a subset of the differences observed. In some embodiments, hypothesis testing for difference may also be applied to output of a functional transformation of assay output. In some embodiments, additional processes other than tests of difference can be used to classify or predict drug response from assay output.

In certain embodiments, the hematological cancer is selected from the group consisting of leukemia, lymphoma, and multiple myeloma. In one embodiment, the hematological cancer is leukemia. In another embodiment, the hematological cancer is lymphoma. In still another embodiment, the hematological cancer is multiple myeloma.

In some embodiments, the lymphoma is DLBCL. In one embodiment, the DLBCL is refractory in the first patient. In another embodiment, the DLBCL is relapsed in the first patient. In yet another embodiment, the DLBCL is a GCB-DLBCL in the first patient. In still another embodiment, the DLBCL is ABC-DLBCL in the first patient. In yet another embodiment, the DLBCL is NOS-DLBCL in the first patient.

In some embodiments, the methods described herein include determining the gene expression profile of a subset(s) of genes in DLBCL patients that have taken the drug prior to treatment with the drug, wherein each subset of genes relates to a tumor biopsy composition.

In certain embodiments, the drug is administered to a DLBCL patient as a dose of from about 0.1 mg per day to about 100 mg per day. In other embodiments, the drug is administered a DLBCL patient as a dose of between about 0.5 mg per day to about 100 mg per day. In other embodiments, the drug is administered a DLBCL patient as a dose of between about 0.5 mg per day to about 20 mg per day. In other embodiments, the drug is administered a DLBCL patient as a dose of between about 5 mg per day to about 25 mg per day. In some embodiments, the drug is administered a DLBCL patient as a dose of between about 0.5 mg per day to about 10 mg per day. In certain embodiments, the drug is administered a DLBCL patient as a dose of between about 0.5 mg per day to about 100 mg per day.

In other embodiments, the drug is administered at a dose of about 0.1 mg, 0.5 mg, 1 mg, 1.5 mg, 2 mg, 2.5 mg, 3 mg, 3.5 mg, 4.0 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 20 mg, 25 mg, 30 mg, 35 mg, 40 mg, 45 mg, 50 mg, 60 mg, 70 mg, 80 mg, 90 mg or 100 mg per day.

In some embodiments, the drug is administered once daily. In some embodiments, the drug is administered twice daily. In certain embodiments, the drug is cyclically administered to a patient with DLBCL. Cycling therapy involves the administration of an active agent for a period of time, followed by a rest for a period of time, and repeating this sequential administration. Accordingly, in some embodiments, about 0.5 mg per day to about 100 mg per day of the drug is administered on days 1-12 of a repeated 28 day cycle. In a specific embodiment, 25 mg of the drug is administered once a day on days 1-12 of a repeated 28 day cycle. In some embodiments, the drug is administered on an intermittent dosing schedule. In a specific embodiment, 2 mg or 3 mg of the drug (e.g., lenalidomide or Compound A) is administered daily for 5 continuous days out of 7 days a week.

It is understood that specific dose levels of a drug described for any particular subject depends upon a variety of factors including the activity of the specific compound employed, the age, body weight, general health, sex, and diet of the patient, the time of administration, the rate of excretion, the drug combination, and the severity of the DLBCL being treated and form of administration. In it also understand that one of ordinary skill in the art can readily determine the appropriate dose of the drug based on these factors. Treatment dosages generally may be titrated to optimize safety and efficacy.

A drug can be administered by any route of administration known in the art such as oral, intravenous, subcutaneous, or intramucosal administration. In one embodiment, lenalidomide or a stereoisomer thereof; or a pharmaceutically acceptable salt, solvate, hydrate, co-crystal, clathrate thereof; or a polymorph thereof, is administered to a DLBCL patient orally. In one embodiment, Compound A or a stereoisomer thereof or a pharmaceutically acceptable salt, solvate, hydrate, co-crystal, clathrate thereof or a polymorph thereof, is administered to a DLBCL patient orally. In some embodiments, a combination of lenalidomide or a stereoisomer thereof; or a pharmaceutically acceptable salt, solvate, hydrate, co-crystal, clathrate thereof; or a polymorph thereof, and Compound A, a stereoisomer thereof; or a pharmaceutically acceptable salt, solvate, hydrate, co-crystal, clathrate thereof; or a polymorph thereof is administered to the DLBCL patient orally. The oral dosage form can be a tablet or a capsule. In some embodiments, the dosage form is a tablet. In some other embodiments, the dosage for is a capsule.

In one embodiment, the drug described herein can be combined with another therapy, such as described in Section 5.5, infra.

### 5.4 Treatment Compounds

5.4.1 3-(4-amino-1-oxo-1,3-dihydro-isoindol-2-yl)-piperidine-2,6-dione (Lenalidomide)

In some embodiments, the compound administered to a DLBCL patient is 3-(4-amino-1-oxo-1,3-dihydro-isoindol-2-yl)-piperidine-2,6-dione (lenalidomide; REVLIMID®), or a stereoisomer thereof; or a pharmaceutically acceptable salt, solvate, hydrate, co-crystal, clathrate thereof; or a polymorph thereof. Such compounds can be formulated for the appropriate route of administration using techniques known in the art.

5.4.2 Preparation of 3-(4-amino-1-oxo-1,3-dihydro-isoindol-2-yl)-piperidine-2,6-dione (Lenalidomide)

Methyl 2-bromomethyl-3-nitrobenzoate

A stirred mixture of methyl 2-methyl-3-nitrobenzoate (14.0 g, 71.7 mmol) and N-bromosuccinimide (15.3 g, 86.1 mmol) in carbon tetrachloride (200 mL) was heated under gentle reflux for 15 hours while a 100 W bulb situated 2 cm away was shining on the flask. The mixture was filtered and the solid was washed with methylene chloride (50 mL). The filtrate was washed with water (2×100 mL), brine (100 mL) and dried. The solvent was removed in vacuo and the residue was purified by flash chromatography (hexane/ethyl acetate, 8/2) to afford 19 g (96%) of the product as a yellow solid: mp 70.0-71.5° C.; 1H NMR (CDCl₃) δ 8.12-8.09 (dd, J=1.3 and 7.8 Hz, 1H), 7.97-7.94 (dd, J=1.3 and 8.2 Hz, 1H), 7.54 (t, J=8.0 Hz, 1H). 5.15 (s, 2H), 4.00 (s, 3H); ¹³C NMR (CDCl₃) δ 165.85, 150.58, 134.68, 132.38, 129.08, 127.80, 53.06, 22.69; HPLC, Water Nove-Pak/C18, 3.9×150 mm, 4 micron, 1 mL/min, 240 nm, 40/60 CH₃CN/0.1% H₃PO₄(aq) 7.27 min(98.92%); Anal. Calcd for C₉H₈NO₄Br: C, 39.44; H, 2.94; N, 5.1 1; Br, 29.15. Found: C, 39.46; H, 3.00; N, 5.00; Br, 29.1 1.

t-Butyl N-(1-oxo-4-nitroisoindolin-2-yl)-L-glutamine

Triethylamine (2.9 g, 28.6 mmol) was added dropwise to a stirred mixture of methyl 2-bromomethyl-3-nitrobenzoate (3.5 g, 13.0 mmol) and L-glutamine t-butyl ester hydrochloride (3.1 g, 13.0 mmol) in tetrahydrofuran (90 mL). The mixture was heated to reflux for 24 hours. To the cooled mixture was added methylene chloride (150 mL) and the mixture was washed with water (2×40 mL), brine (40 mL) and dried. The solvent was removed in vacuo and the residue was purified by flash chromatography (3% CH₃OH in methylene chloride) to afford 2.84 g (60%) of crude product which was used directly in the next reaction: 1H NMR (CDCl₃) δ 8.40 (d, J=8.1 Hz, 1H), 8.15 (d, J=7.5 Hz, 1H), 7.71 (t, J=7.8 Hz, 1H), 5.83 (s, 1H), 5.61 (s, 1H), 5.12 (d, J=19.4 Hz, 1H), 5.04-4.98 (m, 1H), 4.92 (d, J=19.4 Hz, 1H), 2.49-2.22 (m, 4H). 1.46 (s, 9H); HPLC, Waters Nova-Pak C18, 3.9×150 mm, 4 micron, 1 mL/min, 240 nm, 25/75 CH₃CN/0.1% H₃PO₄(aq) 6.75 min(99.94%).

N-(1-oxo-4-nitroisoindolin-2-yl)-L-glutamine

Hydrogen chloride gas was bubbled into a stirred 5° C. solution of t-butyl N-(1-oxo-4-nitro-isoindolin-2-yl)-L-glutamine (3.6 g, 9.9 mmol) in methylene chloride (60 mL) for 1 hour. The mixture was then stirred at room temperature for another hour. Ether (40 mL) was added and the resulting mixture was stirred for 30 minutes. The slurry was filtered, washed with ether and dried to afford 3.3 g of the product: 1H NMR (DMSO-d₆) δ 8.45 (d, J=8.1 Hz, 1H), 8.15 (d, J=7.5 Hz, 1H), 7.83 (t, J=7.9 Hz. 1H), 7.24 (s, 1H), 6.76 (s, 1H), 4.93 (s, 2H), 4.84-4.78 (dd, J=4.8 and 10.4 Hz, 1H), 2.34-2.10 (m, 4H); ¹³C NMR (DMSO-d₆) δ 173.03, 171.88, 165.96, 143.35, 137.49, 134.77, 130.10, 129.61, 126.95, 53.65, 48.13, 31.50, 24.69; Anal. Calcd for C₁₃H₁₃N₃O₆: C, 50.82; H, 4.26; N, 13.68. Found: C, 50.53; H. 4.37; N, 13.22.

(S)-3-(1-oxo-4-nitroisoindolin-2-yl)piperidine-2,6-dione

A stirred suspension mixture of N-(1-oxo-4-nitroisoindolin-2-yl)-L-glutamine (3.2 g, 10.5 mmol) in anhydrous methylene chloride (150 mL) was cooled to −40° C. with isopropanol/dry ice bath. Thionyl chloride (0.82 mL, 11.3 mmol) was added dropwise to the cooled mixture followed by pyridine (0.9 g. 1 1.3 mmol). After 30 min, triethylamine (1.2 g, 11.5 mmol) was added and the mixture was stirred at −30 to −40° C. for 3 hours. The mixture was poured into ice water (200 mL) and the aqueous layer was extracted with methylene chloride (40 mL). The methylene chloride solution was washed with water (2×60 mL), brine (60 mL) and dried. The solvent was removed in vacuo and the solid residue was slurried with ethyl acetate (20 mL) to give 2.2 g (75%) of the product as a white solid: mp 285° C.; 1H NMR (DMSO-d₆) δ: 1.04 (s, 1H), 8.49-8.45 (dd, J=0.8 and 8.2 Hz, 1H), 8.21-8.17 (dd, J=7.3 Hz, 1H), 7.84 (t, J=7.6 Hz, 1H), 5.23-5.15 (dd, J=4.9 and 13.0 Hz, 1H), 4.96 (dd, J=19.3 and 32.4 Hz, 2H), 3.00-2.85 (m, 1H), 2.64-2.49 (m, 2H), 2.08-1.98 (m, 1H); ¹³C NMR (DMSO-d₆) δ 172.79, 170.69, 165.93, 143.33, 137.40, 134.68, 130.15, 129.60, 127.02, 51.82, 48.43, 31.16. 22.23; HPLC, Waters Nove-Pak/C18, 3.9×150 mm, 4 micron, 1 mL/min, 240 nm, 20/80 CH₃CN/0.1% H₃PO₄(aq) 3.67 min(100%); Anal. Calcd for C₁₃HₙN₃O₅: C, 53.98; H, 3.83; N, 14.53. Found: C, 53.92; H, 3.70; N, 14.10.

3-(4-amino-1-oxo-1,3-dihydro-isoindol-2-yl)-piperidine-2,6-dione

A mixture of (S)-3-(1-oxo-4-nitroisoindolin-2-yl)piperidine-2,6-dione (1.0 g, 3.5 mmol) and 10% Pd/C (0.3 g) in methanol (600 mL) was hydrogenated in a Parr-Shaker apparatus at 50 psi of hydrogen for 5 hours. The mixture was filtered through Celite and the filtrate was concentrated in vacuo. The solid was slurried in hot ethyl acetate for 30 min, filtered and dried to afford 0.46 g (51%) of the product as a white solid: mp 235.5-239° C.; ¹H NMR (DMSO-d₆) δ 11.01 (s, 1H). 7.19 (t, J=7.6 Hz, 1H). 6.90 (d. J=7.3 Hz, 1H), 6.78 (d, J=7.8 Hz, 1H), 5.42 (s, 2H). 5.12 (dd. J=5.1 and 13.1 Hz, 1H), 4.17 (dd, J=17.0 and 28.8 Hz, 2H), 2.92-2.85 (m, 1H). 2.64-2.49 (m, 1H). 2.34-2.27 (m, 1H), 2.06-1.99 (m, 1H); ¹³C NMR (DMSO-d₆) δ 172.85, 171.19, 168.84, 143.58, 132.22. 128.79, 125.56, 1 16.37, 1 10.39, 51.48, 45.49, 31.20, 22.74; HPLC. Waters Nova-Pak/C18, 3.9×150 mm, 4 micron, 1 mL/min, 240 nm, 10/90 CH₃CN/0.1% H₃PO₄(aq) 0.96 min(100%); Chiral analysis, Daicel Chiral Pak AD, 40/60 Hexane/IPA, 6.60 min(99.42%); Anal. Calcd for C₁₃H₁₃N₃O₃: C, 60.23; H, 5.05; N, 16.21. Found: C, 59.96; H. 4.98; N, 15.84.

3-(4-Amino-1-oxo-1,3-dihydro-isoindol-2-yl)-piperidine-2,6-dione may also be prepared by methods known in the art, for example, as provided in Drugs of the Future, 2003, 28(5): 425-431, the entirety of which is incorporated by reference.

5.4.3 3-(5-amino-2-methyl-4-oxo-4H-quinazolin-3-yl)-piperidine-2,6-dione (Compound A)

In some embodiments, the compound administered to a patient is 3-(5-amino-2-methyl-4-oxo-4H-quinazolin-3-yl)-piperidine-2,6-dione (“Compound A”), which has the following structure:

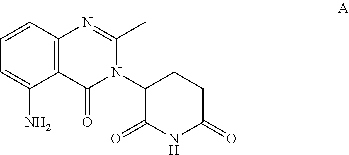 [SMILES: Cc1nc2cccc(N)c2c(=O)n1C1CCC(=O)NC1=O]

or a stereoisomer thereof; or a pharmaceutically acceptable salt, solvate, hydrate, co-crystal, clathrate thereof; or a polymorph thereof. Such compounds can be formulated for the appropriate route of administration using techniques known in the art.

Compound A can be prepared as described in U.S. Pat. No. 7,635,700, the disclosure of which is incorporated herein by reference in its entirety. The compound can be also synthesized according to other methods apparent to those of skill in the art based upon the teaching herein. In certain embodiments, Compound A is in a crystalline form described in U.S. Pat. No. 8,802,685, issued Aug. 12, 2014, which is incorporated herein by reference in its entirety. In some embodiments, the hydrochloride salt of Compound A is used in the methods provided herein. Methods of treating, preventing and/or managing cancers and other diseases using Compound A are described in U.S. Publication No. 2012/0230983, published Sep. 12, 2012, which is incorporated herein by reference in its entirety.

5.4.4 Preparation of Compound A

To a solution of potassium hydroxide (16.1 g, 286 mmol) in water (500 mL), was added 3-nitrophthalimide (25.0 g, 130 mmol) in portion at 0° C. The suspension was stirred at 0° C. for 3 hrs, and then heated to 30° C. for 3 hrs. To the solution, was added HCl (100 mL, 6N). The resulting suspension was cooled to 0° C. for 1 hr. The suspension was filtered and washed with cold water (2×10 mL) to give 3-nitro-phthalamic acid as a white solid (24.6 g, 90% yield): ¹H NMR (DMSO-d₆) δ 7.69 (brs, 1H, NHH), 7.74 (t, J=8 Hz, 1H, Ar), 7.92 (dd, J=1, 8 Hz, 1H, Ar), 8.13 (dd, J=1, 8 Hz, 1H, Ar), 8.15 (brs, 1H, NHH), 13.59 (s, 1H, OH); ¹³C NMR (DMSO-d₆) δ 125.33, 129.15, 130.25, 132.54, 136.72, 147.03, 165.90, 167.31.

To a mixture of 3-nitro-phthalamic acid (24.6 g, 117 mmol) and potassium hydroxide (6.56 g, 117 mmol) in water (118 mL), was added a mixture of bromine (6 mL), potassium hydroxide (13.2 g, 234 mmol) in water (240 mL) at 0° C., followed by addition of a solution of potassium hydroxide (19.8 g, 351 mmol) in water (350 mL). After 5 minutes at 0° C., the mixture was heated in a 100° C. oil bath for 1 hr. The reaction solution was cooled to room temperature, and then, in an ice-water bath for 30 minutes. To the mixture, a solution of HCl (240 mL, 2N) was added dropwise at 0° C., and the resulting mixture was kept for 1 hr. The suspension was filtered and washed with water (5 mL) to give 2-amino-6-nitro-benzoic acid as yellow solid (15.6 g, 73% yield): HPLC: Waters Symmetry C18, 5 μm, 3.9×150 mm, 1 mL/min, 240 nm, CH₃CN/0.1% H₃PO₄, 5% grad to 95% over 5 min, 5.83 min (85%); ¹H NMR (DMSO-d₆) δ 6.90 (dd, J=1, 8 Hz, 1H, Ar), 7.01 (dd, J=1, 9 Hz, 1H, Ar), 7.31 (t, J=8 Hz, 1H, Ar), 8.5-9.5 (brs, 3H, OH, NH₂); ¹³C NMR (DMSO-d₆) δ 105.58, 110.14, 120.07, 131.74, 149.80, 151.36, 166.30; LCMS: MH=183.

A mixture of 2-amino-6-nitro-benzoic acid (1.5 g, 8.2 mmol) in acetic anhydride (15 mL) was heated at 200° C. for 30 minutes in a microwave oven. The mixture was filtered and washed with ethyl acetate (20 mL). The filtrate was concentrated in vacuo. The solid was stirred in ether (20 mL) for 2 hrs. The suspension was filtered and washed with ether (20 mL) to give 2-methyl-5-nitro-benzo[d][1,3]oxazin-4-one as a light brown solid (1.4 g, 85% yield): HPLC: Waters Symmetry C18, 5 μm, 3.9×150 mm, 1 mL/min, 240 nm, CH₃CN/0.1% H₃PO₄, 5% grad 95% in 5 min, 5.36 min (92%); ¹H NMR (DMSO-d₆) δ 2.42 (s, 3H, CH₃), 7.79 (dd, J=1, 8 Hz, 1H, Ar), 7.93 (dd, J=1, 8 Hz, 1H, Ar), 8.06 (t, J=8 Hz, 1H, Ar); ¹³C NMR (DMSO-d₆) δ 20.87, 107.79, 121.54, 128.87, 137.19, 147.12, 148.46, 155.18, 161.78; LCMS: MH=207.

Two vials each with a suspension of 5-nitro-2-methyl-benzo[d][1,3]oxazin-4-one (0.60 g, 2.91 mmol) and 3-amino-piperidine-2,6-dione hydrogen chloride (0.48 g, 2.91 mmol) in pyridine (15 mL) were heated at 170° C. for 10 minutes in a microwave oven. The suspension was filtered and washed with pyridine (5 mL). The filtrate was concentrated in vacuo. The resulting mixture was stirred in HCl (30 mL, 1N), ethyl acetate (15 mL) and ether (15 mL) for 2 hrs. The suspension was filtered and washed with water (30 mL) and ethyl acetate (30 mL) to give a dark brown solid, which was stirred with methanol (50 mL) at room temperature overnight. The suspension was filtered and washed with methanol to give 3-(2-methyl-5-nitro-4-oxo-4H-quinazolin-3-yl)-piperidine-2,6-dione as a black solid (490 mg, 27% yield). The solid was used in the next step without further purification.

A mixture of 3-(2-methyl-5-nitro-4-oxo-4H-quinazolin-3-yl)-piperidine-2,6-dione (250 mg) and Pd(OH)₂ on carbon (110 mg) in DMF (40 mL) was shaken under hydrogen (50 psi) for 12 hrs. The suspension was filtered through a pad of Celite and washed with DMF (10 mL). The filtrate was concentrated in vacuo and the resulting oil was purified by flash column chromatography (silica gel, methanol/methylene chloride) to give 3-(5-amino-2-methyl-4-oxo-4H-quinazolin-3-yl)-piperidine-2,6-dione as a white solid (156 mg, 69% yield): HPLC: Waters Symmetry C18, 5 μm, 3.9×150 mm, 1 mL/min, 240 nm, 10/90 CH₃CN/0.1% H₃PO₄, 3.52 min (99.9%); mp: 293-295° C.; ¹H NMR (DMSO-d₆) δ 2.10-2.17 (m, 1H, CHH), 2.53 (s, 3H, CH₃), 2.59-2.69 (m, 2H, CH₂), 2.76-2.89 (m, 1H, CHH), 5.14 (dd, J=6, 11 Hz, 1H, NCH), 6.56 (d, J=8 Hz, 1H, Ar), 6.59 (d, J=8 Hz, 1H, Ar), 7.02 (s, 2H, NH₂), 7.36 (t, J=8 Hz, 1H, Ar), 10.98 (s, 1H, NH); ¹³C NMR (DMSO-d₆) δ 20.98, 23.14, 30.52, 55.92, 104.15, 110.48, 111.37, 134.92, 148.17, 150.55, 153.62, 162.59, 169.65, 172.57; LCMS: MH=287; Anal. Calcd. for C₁₄H₁₄N₄O₃+0.3 H₂O: C, 57.65; H, 5.05; N, 19.21. Found: C, 57.50; H, 4.73; N, 19.00.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.5 Combination Therapy

One or more additional therapies, such as additional active ingredients or agents, that can be used in combination with the administration of a drug described herein to treat a DLBCL patient. In a specific embodiment, one or more additional active ingredients or agents can be used in the methods provided herein with a drug. The one or more additional therapies can be administered prior to, concurrently with, or subsequent to the administration of a drug described herein. Administration of a drug described herein and an additional active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated. Routes of administration for the additional active agents or ingredients are known to those of ordinary skill in the art. See, e.g., Physicians' Desk Reference.

In certain embodiments, a drug described herein and an additional active agent are cyclically administered to a patient with a hematological cancer (e.g., DLBCL). Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

The additional active agents administered in combination with a drug described herein can be large molecules (e.g., proteins) or small molecules (e.g., synthetic inorganic, organometallic, or organic molecules). In certain embodiments, the additional active agent is an immunomodulatory therapy. In other embodiments, the additional active agent is not a drug described herein. Examples of large molecule active agents include, but are not limited to, hematopoietic growth factors, cytokines, and monoclonal and polyclonal antibodies. In certain embodiments, large molecule active agents are biological molecules, such as naturally occurring or artificially made proteins. Proteins that are useful include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and compositions of the disclosure include, but are not limited to: filgrastim, which is sold in the United States under the trade name NEUPOGEN® (Amgen, Thousand Oaks, Calif.); sargramostim, which is sold in the United States under the trade name LEUKINE® (Immunex, Seattle, Wash.); and recombinant EPO, which is sold in the United States under the trade name EPGEN® (Amgen, Thousand Oaks, Calif.).

Inhibitors of ActRII receptors or activin-ActRII inhibitors may be used in the methods and compositions provided herein. ActRII receptors include ActRIIA inhibitors and ActRIIB inhibitors. Inhibitors of ActRII receptors can be polypeptides comprising activin-binding domains of ActRII. In certain embodiments, the activin-binding domain comprising polypeptides are linked to an Fc portion of an antibody (i.e., a conjugate comprising an activin-binding domain comprising polypeptide of an ActRII receptor and an Fc portion of an antibody is generated). In certain embodiments, the activin-binding domain is linked to an Fc portion of an antibody via a linker, e.g., a peptide linker. Examples of such non-antibody proteins selected for activin or ActRIIA binding and methods for design and selection of the same are found in WO/2002/088171, WO/2006/055689, WO/2002/032925, WO/2005/037989, US 2003/0133939, and US 2005/0238646, each of which is incorporated herein by reference in its entirety.

Recombinant and mutated forms of GM-CSF can be prepared as described in U.S. Pat. Nos. 5,391,485; 5,393,870; and 5,229,496; the disclosure of each of which is incorporated herein by reference in its entirety. Recombinant and mutated forms of G-CSF can be prepared as described in U.S. Pat. Nos. 4,810,643; 4,999,291; 5,528,823; and 5,580,755; the disclosure of each of which is incorporated herein by reference in its entirety.

This disclosure encompasses the use of native, naturally occurring, and recombinant proteins. The disclosure further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with a drug described herein include monoclonal and polyclonal antibodies. Examples of antibodies include, but are not limited to, trastuzumab (HERCEPTIN), rituximab (RITUXAN®), bevacizumab (AVASTIN®), pertuzumab (OMNITARG™), tositumomab (BEXXAR®), edrecolomab (PANOREX®), panitumumab and G250. An immunomodulatory therapy provided herein can also be combined with or used in combination with anti-TNF-alpha antibodies.

Large molecule active agents may be administered in the form of anti-cancer vaccines. For example, vaccines that secrete, or cause the secretion of, cytokines such as IL-2, SCF, CXC14 (platelet factor 4), G-CSF, and GM-CSF can be used in the methods, pharmaceutical compositions, and kits of the disclosure. See, e.g., Emens, L. A., et al., Curr. Opinion Mol. Ther. 3(1):77-84 (2001).

Additional active agents that are small molecules can also be used to alleviate adverse effects associated with the administration of a drug described herein. However, like some large molecules, many are believed to be capable of providing a synergistic effect when administered with (e.g., before, after or simultaneously) the immunomodulatory therapy. Examples of small molecule additional active agents include, but are not limited to, anti-cancer agents, antibiotics, immunosuppressive agents, and steroids.

Examples of anti-cancer agents include, but are not limited to: abraxane; ace-11; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porflmer sodium; porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; stem cell treatments such as PDA-001; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; b-FGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®.), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O₆-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific additional active agents include, but are not limited to, oblimersen (GENASENSE®), remicade, docetaxel, celecoxib, melphalan, dexamethasone (DECADRON®), steroids, gemcitabine, cisplatinum, temozolomide, etoposide, cyclophosphamide, temodar, carboplatin, procarbazine, gliadel, tamoxifen, topotecan, methotrexate, ARISA®., taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (DOXIL®), paclitaxel, ganciclovir, adriamycin, estramustine sodium phosphate (EMCYT), sulindac, and etoposide.

### 5.6 Biological Samples

In certain embodiments, the various methods provided herein use samples (e.g., biological samples) from patients. The patient can be male or female, and can be an adult, child or infant. Samples can be analyzed at a time during an active phase of DLBCL, or when DLBCL is inactive. In one embodiment, a sample is obtained from a patient prior, concurrently with and/or subsequent to administration of a drug described herein. In a specific embodiment, a sample is obtained from a patient prior to administration of a drug described herein. In certain embodiments, more than one sample from a patient can be obtained.

In certain embodiments, the sample used in the methods provided herein comprises body fluids from a subject. Non-limiting examples of body fluids include blood (e.g., peripheral whole blood, peripheral blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids, including cerebrospinal fluid surrounding the brain and the spinal cord, synovial fluid surrounding bone joints, intracellular fluid, and vitreous humour. In some embodiments, the sample is a blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, B cells, T cells, monocytes, granulocytes or lymphocytes, can be further isolated using conventional techniques, e.g. magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0 or 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. In a specific embodiment, the sample used in the methods described herein comprises a tumor biopsy. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the patient receiving a treatment for DLBCL. In another embodiment, the sample is obtained from the patient during the subject receiving a treatment for the DLBCL. In another embodiment, the sample is obtained from the patient after the patient received a treatment for the DLBCL. In various embodiments, the treatment comprises administering a drug described herein (e.g., see Section 5.4) to the subject.

In certain embodiments, the sample used in the methods provided herein comprises a plurality of cells. Such cells can include any type of cells, e.g., stem cells, blood cells (e.g., peripheral blood mononuclear cells), lymphocytes, B cells, T cells, monocytes, granulocytes, immune cells, or tumor or cancer cells. The tumor or cancer cells or a tumor tissue, such as a tumor biopsy or a tumor explants. T cells (T lymphocytes) include, for example, helper T cells (effector T cells or Th cells), cytotoxic T cells (CTLs), memory T cells, and regulatory T cells. In one embodiment, the cells used in the methods provided herein are CD3⁺ T cells, e.g., as detected by flow cytometry. The number of T cells used in the methods can range from a single cell to about 10⁹ cells. B cells (B lymphocytes) include, for example, plasma B cells, dendritic cells, memory B cells, B1 cells, B2 cells, marginal-zone B cells, and follicular B cells. B cells can express immunoglobulins (antibodies, B cell receptor).

Specific cell populations can be obtained using a combination of commercially available antibodies (e.g., Quest Diagnostic (San Juan Capistrano, Calif.); Dako (Denmark)).

In certain embodiments, the sample used in the methods provided herein is from a diseased tissue from a DLBCL patient. In certain embodiments, the number of cells used in the methods provided herein can range from a single cell to about 10⁹ cells. In some embodiments, the number of cells used in the methods provided herein is about 1×10⁴, 5×10⁴, 1×10⁵, 5×10⁵, 1×10⁶, 5×10⁶, 1×10⁷, 5×10⁷, 1×10⁸, or 5×10⁸.

The number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In certain embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc.

### 5.7 Methods for Detecting RNA Expression

Several methods of detecting or quantitating mRNA levels are known in the art. Exemplary methods include but are not limited to northern blots, ribonuclease protection assays, PCR-based methods, and the like. The mRNA sequence can be used to prepare a probe that is at least partially complementary. The probe can then be used to detect the mRNA sequence in a sample, using any suitable assay, such as PCR-based methods, Northern blotting, a dipstick assay, and the like.

In other embodiments, a nucleic acid assay for testing for immunomodulatory activity in a biological sample can be prepared. An assay typically contains a solid support and at least one nucleic acid contacting the support, where the nucleic acid corresponds to at least a portion of an mRNA encoded by a gene listed in Table 1, Table 2, Table 3, or Table 4. The assay can also have a means for detecting the altered expression of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

Any suitable assay platform can be used to determine the presence of the mRNA in a sample. For example, an assay may be in the form of a dipstick, a membrane, a chip, a disk, a test strip, a filter, a microsphere, a slide, a multiwell plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label. Exemplary fluorescent dyes include but are not limited to xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, JOE, R6G, Tetramethylrhodamine, Lissamine, ROX, Napthofluorescein, and the like.

In some embodiments, the mRNA sequences comprise at least one mRNA selected from the mRNAs encoded by the genes listed in Table 1, Table 2, Table 3, or Table 4, or a fragment thereof. The nucleic acids may be present in specific, addressable locations on a solid support; each corresponding to at least a portion of mRNA sequences that are differentially expressed upon treatment of an immunomodulatory compound in a cell or a patient.

A typical mRNA assay method can contain the steps of 1) obtaining surface-bound subject probes; 2) hybridization of a population of mRNAs to the surface-bound probes under conditions sufficient to provide for specific binding (3) post-hybridization washes to remove nucleic acids not bound in the hybridization; and (4) detection of the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g. under conditions sufficient to provide for specific binding of target mRNAs in the sample to the probes) are described in Kallioniemi et al., Science 258:818-821 (1992) and WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to follow the expression of the genes in Table 1, Table 2, Table 3, or Table 4. Examples of PCR methods can be found in the literature. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantitation of RNA targets (Bustin, et al., 2005, Clin. Sci., 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, real-time PCR gives quantitative results. An additional advantage of real-time PCR is the relative ease and convenience of use. Instruments for real-time PCR, such as the Applied Biosystems 7500, are available commercially, as are the reagents, such as TaqMan Sequence Detection chemistry. For example, TaqMan® Gene Expression Assays can be used, following the manufacturer's instructions. These kits are pre-formulated gene expression assays for rapid, reliable detection and quantification of human, mouse and rat mRNA transcripts. An exemplary PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using a 7500 Real-Time PCR System Sequence Detection software v1.3 using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

Techniques known to one skilled in the art may be used to measure the amount of an RNA transcript(s). In some embodiments, the amount of one, two, three, four, five or more RNA transcripts is measured using deep sequencing, such as ILLUMINA® RNASeq, ILLUMINA® next generation sequencing (NGS), ION TORRENT′ RNA next generation sequencing, 454′ pyrosequencing, or Sequencing by Oligo Ligation Detection (SOLID™). In other embodiments, the amount of multiple RNA transcripts is measured using a microarray and/or gene chip. In certain embodiments, the amount of one, two, three or more RNA transcripts is determined by RT-PCR. In other embodiments, the amount of one, two, three or more RNA transcripts is measured by qRT-PCR. Techniques for conducting these assays are known to one skilled in the art. In yet other embodiments, NanoString (e.g., nCounter® miRNA Expression Assays provided by NanoString® Technologies) is used for analyzing RNA transcripts.

### 5.8 Methods for Detecting Protein Expression

Several protein detection and quantitation methods can be used to measure the level of proteins. Any suitable protein quantitation method can be used. In some embodiments, antibody-based methods are used. Exemplary methods that can be used include but are not limited to immunoblotting (western blot), enzyme-linked immunosorbent assay (ELISA), immunohistochemistry, flow cytometry, cytometric bead array, mass spectroscopy, and the like. Several types of ELISA are commonly used, including direct ELISA, indirect ELISA, and sandwich ELISA.

### 5.9 Kits

In one aspect, provided herein is a kit for predicting the responsiveness of a patient having a hematological cancer to a treatment compound, comprising an agent for determining the expression levels of the genes identified in Tables 1, 2, 3, and/or 4, or a subset thereof in a biological sample obtained from the patient prior to the compound treatment, wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. In one embodiment, the kit comprises an agent for determining the expression levels of the genes identified in Table 1. In another embodiment, the kit comprises an agent for determining the expression levels of the genes identified in Table 2. In yet another embodiment, the kit comprises an agent for determining the expression levels of the genes identified in Table 3. In still another embodiment, the kit comprises an agent for determining the expression levels of the genes identified in Table 4. In yet another embodiment, the kit comprises an agent for determining the expression levels of the genes identified in Tables 1, 2, 3, and 4.

In another aspect, provided herein is a kit for treating a patient having a hematological cancer with a treatment compound, comprising an agent for determining the expression levels of the genes identified in Tables 1, 2, 3, and/or 4, or a subset thereof in a biological sample obtained from the patient prior to the compound treatment, wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof. In one embodiment, the kit comprises an agent for determining the expression levels of the genes identified in Table 1. In another embodiment, the kit comprises an agent for determining the expression levels of the genes identified in Table 2. In yet another embodiment, the kit comprises an agent for determining the expression levels of the genes identified in Table 3. In still another embodiment, the kit comprises an agent for determining the expression levels of the genes identified in Table 4. In yet another embodiment, the kit comprises an agent for determining the expression levels of the genes identified in Tables 1, 2, 3, and 4.

In one aspect, provided herein are pharmaceutical or assay kits comprising a drug described herein (see, Section 5.4) or a pharmaceutical composition thereof, in one or more containers, and instructions for use. In certain embodiments, the kits useful for predicting the responsiveness of a DLBCL patient to a drug described herein. In further embodiments, the drug, in a container, is accompanied by an apparatus or apparati necessary for administering the drug or composition thereof to a subject. In some embodiments, the instructions provided herein provide a score or threshold level of expression or an output of a functional transformation applied to expression that a gene or subset of genes needs to be achieved in order to indicate that the DLBCL patient will be responsive to the drug described herein.

In certain embodiments, a kit comprises a drug described herein or pharmaceutical composition thereof, in a container, and a reagent or reagents necessary for carrying out an assay(s) described herein, in one or more other containers. In certain embodiments, the kit comprises a solid support, and a means for detecting the RNA or protein expression of at least one biomarker (e.g., a differentially expressed gene identified in Table 1, 2, 3, or 4) in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide.

In a specific embodiment, the pharmaceutical or assay kit comprises, in a container, a drug described herein or a pharmaceutical composition thereof, and further comprises, in one or more containers, components for isolating RNA. In another specific embodiment, the pharmaceutical or assay kit comprises, in a container, a drug described herein or a pharmaceutical composition, and further comprises, in one or more containers, components for conducting RT-PCR, qRT-PCR, deep sequencing, or a microarray such as NanoString assay. In some embodiments, the kit comprises a solid support, nucleic acids contacting the support, where the nucleic acids are complementary to at least 20, 50, 100, 200, 350, or more bases of mRNA, and a means for detecting the expression of the mRNA in a biological sample.

In another specific embodiment, the pharmaceutical or assay kit comprises, in a container, a drug described herein or a pharmaceutical composition thereof, and further comprises, in one or more containers, components for isolating protein In another specific embodiment, the pharmaceutical or assay kit comprises, in a container, a drug described herein or a pharmaceutical composition, and further comprises, in one or more containers, components for conducting flow cytometry or an ELISA.

In another aspect, provided herein are kits for measuring biomarkers providing the materials necessary to measure the abundance of one or more of the gene products of the genes or a subset of genes (e.g., one, two, three, four, five or more genes) in Table 1, 2, 3, or 4, or any combination thereof. Such kits may comprise materials and reagents required for measuring RNA or protein. In some embodiments, such kits include microarrays, wherein the microarray is comprised of oligonucleotides and/or DNA and/or RNA fragments which hybridize to one or more of the products of one or more of the genes or a subset of genes in Table 1, 2, 3, or 4, or any combination thereof. In some embodiments, such kits may include primers for PCR of either the RNA product or the cDNA copy of the RNA product of the genes or subset of genes, or both. In some embodiments, such kits may include primers for PCR as well as probes for qPCR. In some embodiments, such kits may include multiple primers and multiple probes wherein some of said probes have different flourophores so as to permit multiplexing of multiple products of a gene product or multiple gene products. In some embodiments, such kits may further include materials and reagents for creating cDNA from RNA. In some embodiments, such kits may include antibodies specific for the protein products of a gene or subset of genes in Table 1, 2, 3, or 4, or any combination thereof. Such kits may additionally comprise materials and reagents for isolating RNA and/or proteins from a biological sample. In addition such kits may include materials and reagents for synthesizing cDNA from RNA isolated from a biological sample. In some embodiments, such kits may include, a computer program product embedded on computer readable media for predicting whether a patient is responsive to a drug as described herein. In some embodiments, the kits may include a computer program product embedded on a computer readable media along with instructions.

In some embodiments, kits for measuring the expression of one or more nucleic acid sequences of a gene or a subset of genes in Tables 1, 2, 3, and/or 4, or a combination thereof. In a specific embodiment, such kits measure the expression of one or more nucleic acid sequences associated with a gene or a subset of genes in Tables 1, 2, 3, and/or 4, or a combination thereof. In accordance with this embodiment, the kits may comprise materials and reagents that are necessary for measuring the expression of particular nucleic acid sequence products of genes or a subset of genes in Tables 1, 2, 3, and/or 4, or a combination thereof. For example, a microarray or RT-PCR kit may be produced for a specific condition and contain only those reagents and materials necessary for measuring the levels of specific RNA transcript products of the genes or a subset of genes in Tables 1, 2, 3, and/or 4, or a combination thereof to predict whether a DLBCL patient is response to treatment with a drug described herein. Alternatively, in some embodiments, the kits can comprise materials and reagents that are not limited to those required to measure the expression of particular nucleic acid sequences of any particular gene in Tables 1, 2, 3, and/or 4, or a combination thereof. For example, in certain embodiments, the kits comprise materials and reagents necessary for measuring the levels of expression of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, or 75 of the genes in Table 1, 2, 3, or 4, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, or more genes other than those in Tables 1, 2, 3, and/or 4. In other embodiments, the kits contain reagents and materials necessary for measuring the levels of expression of at least 1, at least 2, at least 3, at least 4, at least 5, at least 6, at least 7, at least 8, at least 9, at least 10, at least 11, at least 12, at least 13, at least 14, at least 15, at least 15, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, or more of the genes in Tables 1, 2, 3, and/or 4, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are genes not in Tables 1, 2, 3, and/or 4, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are genes not in Tables 1, 2, 3 and/or 4.

For nucleic acid microarray kits, the kits generally comprise probes attached to a solid support surface. In one such embodiment, probes can be either oligonucleotides or longer length probes including probes ranging from 150 nucleotides in length to 800 nucleotides in length. The probes may be labeled with a detectable label. In a specific embodiment, the probes are specific for one or more of the gene products in Tables 1, 2, 3 and/or 4. The microarray kits may comprise instructions for performing the assay and methods for interpreting and analyzing the data resulting from the performance of the assay. In a specific embodiment, the kits comprise instructions for predicting whether a DLBCL patient is responsive to treatment with a drug described herein. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

In certain embodiments, a nucleic acid microarray kit comprises materials and reagents necessary for measuring the expression levels of 1, 2, 3, 4, 5, 6, 7, 8, 9, 10, 15, 20, 25, 30, 35, 40, 45, 50, or more of the genes in Table 1, 2, 3, or 4, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those in Table 1, 2, 3, or 4. In other embodiments, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes in Table 1, 2, 3, or 4, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not in Table 1, 2, 3, or 4. In another embodiment, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes in Table 1, 2, 3, or 4, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100 1000, or 500-1000 genes that are not in Table 1, 2, 3, or 4.

For qPCR, the kits generally comprise pre-selected primers specific for particular nucleic acid sequences. The qPCR kits may also comprise enzymes suitable for amplifying nucleic acids (e.g., polymerases such as Taq polymerase), deoxynucleotides, and buffers needed for amplification reaction. The qPCR kits may also comprise probes specific for the nucleic acid sequences associated with or indicative of a condition. The probes may or may not be labeled with a fluorophore. The probes may or may not be labeled with a quencher molecule. In some embodiments, the qPCR kits can be qRT-PCT kits, which comprise components suitable for reverse-transcribing RNA, including enzymes (e.g., reverse transcriptases such as AMV, MMLV, and the like) and primers for reverse transcription along with deoxynucleotides and buffers needed for reverse transcription reaction. Each component of the qPCR kit is generally in its own suitable container. Thus, these kits generally comprise distinct containers suitable for each individual reagent, enzyme, primer and probe. Further, the qPCR kits may comprise instructions for performing the reaction and methods for interpreting and analyzing the data resulted from performing the reaction. In a specific embodiment, the kits contain instructions for predicting whether a patient having a hematological cancer (e.g., DLBCL) is clinically sensitive to a compound.

In certain embodiments, a qPCR kit comprises materials and reagents necessary for measuring the expression levels of 1, 2, 3, 4, 5, 6, 7, 8, 9, 10, 15, 20, 25, 30, 35, 40, 45, 50, or more of the genes in Table 1, 2, 3, or 4, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those in Table 1, 2, 3, or 4. In other embodiments, a qPCR kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes in Table 1, 2, 3, or 4, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not in Table 1, 2, 3, or 4. In another embodiment, a qPCR kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes in Table 1, 2, 3, or 4, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100 1000, or 500-1000 genes that are not in Table 1, 2, 3, or 4.

For antibody-based kits, the kit can comprise, for example: (1) a first antibody (which may or may not be attached to a solid support) that binds to a first region of a peptide, polypeptide, or protein of interest; and, optionally, (2) a second antibody that binds to either the first antibody or a second region of the peptide, polypeptide, or protein, and is conjugated to a detectable label (e.g., a fluorescent label, radioactive isotope, or enzyme). In a specific embodiment, the peptide, polypeptide, or protein of interest is associated with or indicative of a condition (e.g., a disease). The antibody-based kits may also comprise beads for conducting immunoprecipitation. Each component of the antibody-based kits is generally in its own suitable container. Thus, these kits generally comprise distinct containers suitable for each antibody and reagent. Further, the antibody-based kits may comprise instructions for performing the assay and methods for interpreting and analyzing the data resulted from performing the assay. In a specific embodiment, the kits contain instructions for predicting whether a hematological cancer in a patient is clinically sensitive to a compound.

In certain embodiments, an antibody-based kit comprises materials and reagents necessary for measuring the expression levels of 1, 2, 3, 4, 5, 6, 7, 8, 9, 10, 15, 20, 25, 30, 35, 40, 45, 50, or more of the genes in Table 1, 2, 3, or 4, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those in Table 1, 2, 3, or 4. In other embodiments, an antibody-based kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes in Table 1, 2, 3, or 4, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not in Table 1, 2, 3, or 4. In another embodiment, an antibody-based kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes in Table 1, 2, 3, or 4, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000, or 500-1000 genes that are not in Table 1, 2, 3, or 4.

### 5.10 Examples

Non-limiting examples provided herein demonstrate the feasibility of applying an algorithm (e.g., a linear model) to a clinically viable platform (e.g., Nanostring or next-generation sequencing), which can be used for the diagnostic.

Initial development of the classifier resulted in a set of 26 genes modeled to produce a binary classification with demonstrated ability to segregate tumors according to their sensitivity to the drug. A proof of principle study was designed to translate the biomarker content and model to a clinically viable platform. The study consisted of four major steps: 1) development of a simplified linear model of the primary signal, 2) placing the model in the scale of a clinically viable technology (such as but not limited to, Nanostring nCounter or qRT-PCR), 3) establishing a threshold to replicate the output of the research assay, and 4) evaluating the simplified model and threshold in the Compound A cohort.

5.10.1 Linear Model Development

The primary goal of the first stage of development was to construct a linear model of gene expression values that would recapitulate the information provided by the research classifier. The Elastic-Net algorithm was selected to learn weights of the linear model. The gene expression dataset comprising transcriptional profiles from biopsy samples of 414 DLBCL patients (Lenz et al., N. Engl. J. Med. 2008, 359(22):2313-2323) was used for development of the linear model. This is the same cohort used during development of the prior 26 gene research model, and was processed in the same form (fRMA summarization followed by COMBAT batch correction and annotation by Entrez Gene ID). Genes available for inclusion in the linear model were limited to a subset known to provide reliable measures on a clinically viable assay. The probabilities output from the research classifier on this cohort were used as the outcome to train the linear model.

Prior to initiating the training exercise, ⅓ of the Lenz cohort was randomized to a validation set and thus not used during training. The remaining ⅔ of training samples were subjected to Elastic-Net linear regression with all predictors (genes) as additive terms and the logit transformed probabilities assigned from the research classifier as the outcome. In the Elastic-Net logistic regression, 2 parameters (alpha and lambda) were optimized. Optimization of parameters was performed using 500 rounds of Monte Carlo cross-validation. Sampling was biased to match the response frequency in the training and test sets of each round. During each iteration of Monte Carlo cross-validation, an Elastic-Net model was built using two-thirds of the training set with the remaining one-third of the training set used for model testing. During each round, performance was evaluated using the test cases based on the trained linear model. The Pearson correlation between the predictions and the known outcomes was assessed. Final parameters were then used to construct a model from the entire training set, and this model was evaluated in the validation set. The cross-validation approach ensured that model training used robust techniques so that the estimated performance of a specified model would be in line with the actual performance during the blinded validation.

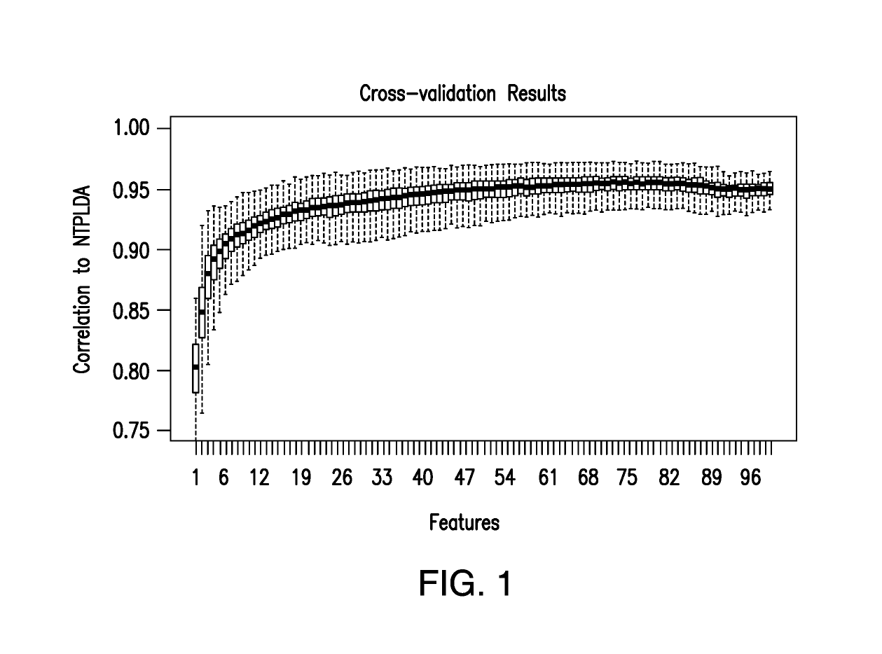

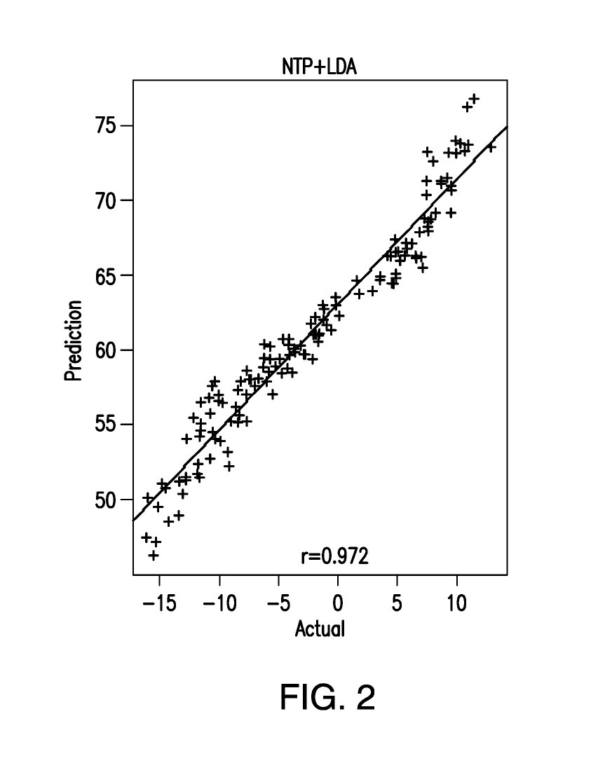

Performance during Monte Carlo cross-validation illustrated a plateau, demonstrating a correlation of 0.95 with the outcomes using parameters that assigned non-zero weights to approximately 50 genes (FIG. 1). These parameters were then used to determine the final model of this stage from the complete training set. The model was used to evaluate the validation set, and performance was established by comparison to the outcome from the research classifier. The resulting performance of the model during validation comprised a correlation of 0.972 (FIG. 2).

5.10.2 Rescaling the Linear Model

The first stage of bridging established that a linear model learned by the Elastic-Net can accurately reproduce the outcome from the research classifier. The next goal was to rescale the model to be in the space of a target clinical assay.

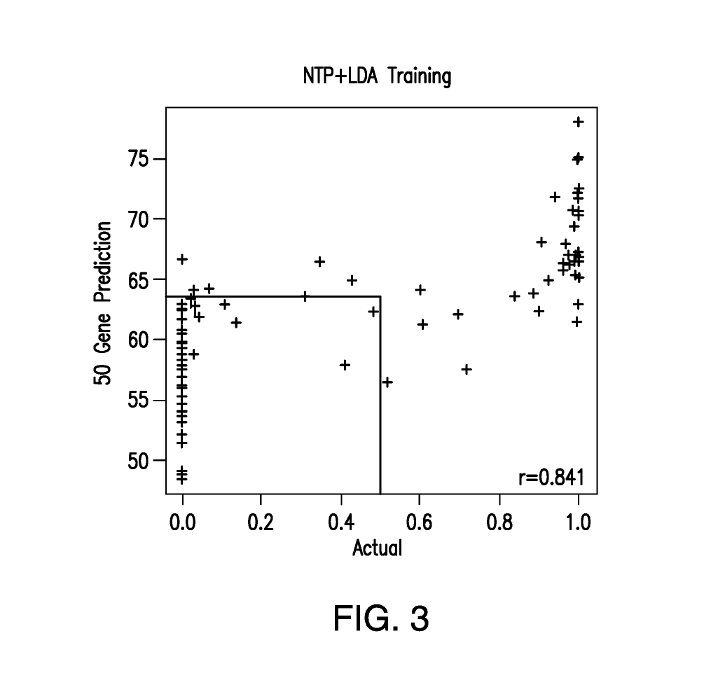

A cohort of 85 Nanostring nCounter profiles from the Compound A cohort were used to rescale the assay. The mean and standard deviation of each gene was estimated from the Nanostring data, and separately for the Lenz cohort. The Lenz data were then rescaled such that the mean and standard deviation of each gene were equivalent to the mean and standard deviation on the Nanostring platform. The Elastic-Net was again fit to the Lenz training set after this transformation using parameters that resulted in the same gene set size optimized during Monte Carlo cross-validation. Performance of the rescaled model was estimated from the Lenz validation set and was unchanged relative to the original result. The final rescaled linear model is presented in FIG. 3.

5.10.3 Threshold Translation

The third stage of bridging was conducted to translate the threshold from the research model such that the linear model output from bridging could be transformed to a binary classification. A linear model was used to fit the logit transformed gold standard outcomes on the Lenz cohort to the predicted values from the linear model finalized in stage 2 of this Bridging study. A value of zero (logit transformed probability of 0.5) was interpolated from this fit to produce the final threshold of the rescaled linear model. The plot of data in their natural scales along with the interpolated threshold is demonstrated in Table 5.

TABLE 5

| Gene | Model Weight |
|---|---|
| BICC1 | 0.139436 |
| C10orf54 | 0.562099 |
| C1RL | 0.512357 |
| C20orf112 | 0.12179 |
| C8orf4 | 0.234008 |
| CCDC88C | −0.21187 |
| CD97 | 0.124489 |
| CEBPB | 0.405436 |
| CFB | 0.168781 |
| CHRDL1 | 0.119651 |
| CIRH1A | −0.42924 |
| CPSF3 | −0.07467 |
| CSF1R | 0.031231 |
| DDR2 | 0.152434 |
| DLGAP5 | −0.00036 |
| DUSP1 | 0.028789 |
| EMR2 | 0.08751 |
| EPB41L3 | 0.51154 |
| FBXO32 | 0.270714 |
| GAS2L1 | 0.265213 |
| GINS4 | −0.23158 |
| IFITM2 | 0.062428 |
| KCNMB1 | 0.094899 |
| LGALS3BP | 0.052791 |
| LRP11 | 0.40935 |
| MEGF6 | 0.506004 |
| MEIS1 | 0.019038 |
| MYO1F | 0.317676 |
| PDGFC | 0.309273 |
| PRNP | 0.079546 |
| PTPRM | 0.07177 |
| RAB31 | 0.206324 |
| RBBP8 | −0.06427 |
| RUVBL1 | −0.02008 |
| SEMA3C | 0.129259 |
| SERPING1 | 0.509476 |
| SMOC2 | 0.171667 |
| SNRPD1 | −0.16953 |
| TBC1D2B | 0.790875 |
| THEMIS2 | 0.15426 |
| TMEM173 | 0.354952 |
| TPSAB1 | 0.132704 |
| TRIP13 | −0.04799 |
| TXNIP | 0.115142 |
| ULK1 | 0.511509 |
| WDR12 | −0.24471 |
| WWTR1 | 0.122723 |
| XAF1 | 0.472417 |
| ZNF215 | −0.23349 |

5.10.4 Clinical Evaluation

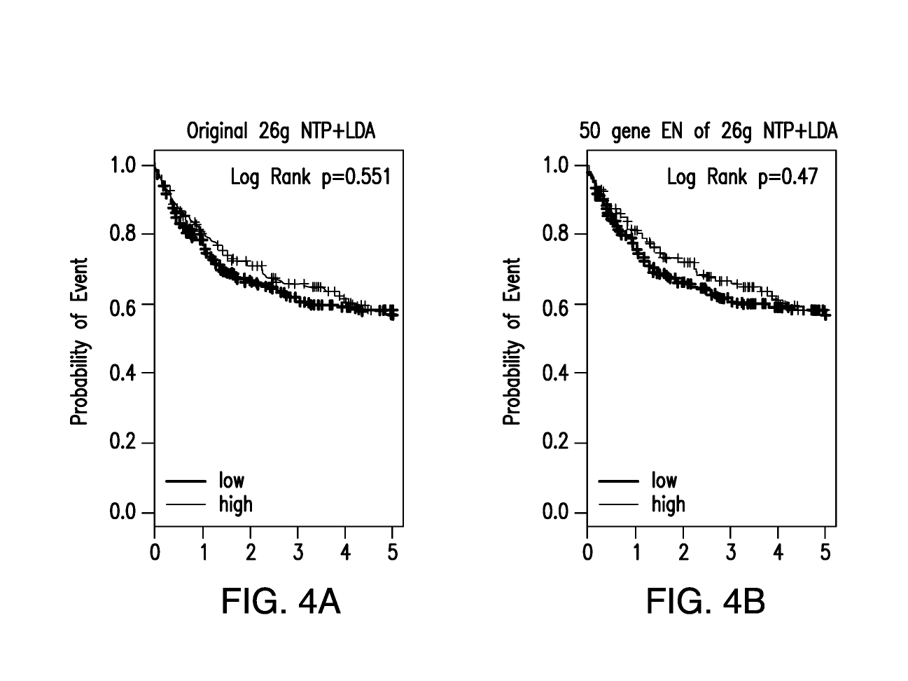

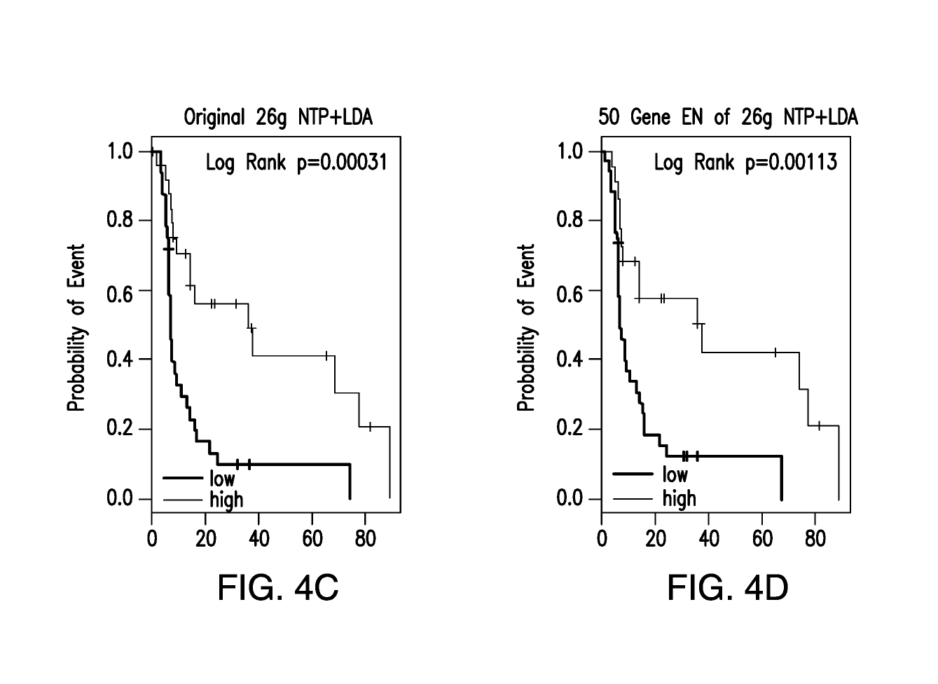

The final model was applied to the Lenz microarray cohort and the Compound A Non-transformed cohort assayed by Nanostring nCounter. The classification of each sample was then combined with clinical data, and associations with clinical variables such as objective response and progression free survival were calculated. Results are shown as Kaplan-Meier plots in FIGS. 4A-4D. FIGS. 4A and 4B demonstrate the performance in the Lenz cohort using overall survival as the outcome. FIGS. 4C and 4D demonstrate performance in the Compound A cohort using progression free survival as the outcome. FIGS. 4A and 4C show the original 26 gene research classifier, and FIGS. 4B and 4D show the linear model that resulted from this bridging study. Results indicate expected performance with no prognostic association in the Lenz cohort, but strong segregation of response in the Compound A cohort. Importantly, the shapes of the curves for each group are very similar between the original 26 gene classifier and the bridged linear model. Additional results including biomarker prevalence, summary statistics from Cox Proportional Hazards models, objective response, and model details are listed in Table 6.

TABLE 6

| | Feature | CG26 | M1 | Data Source |
|---|---|---|---|---|
| Biomarker | Prev | 43.9% | 38.6% | Cmp A-ST |
| Prevalence | BM+ | 25 | 22 | (N = 57) |
| | BM− | 32 | 35 | |
| Predictive | Cox HR | 0.31 | 0.32 | |
| Effects | Cox p | 0.0006 | 0.0019 | |
| | Median Surv | 6.7 | 6.9 | |
| | BM− | | | |
| | Median Surv | 35.9 | 37.9 | |
| | BM+ | | | |
| | CR BM+ | 4 | 4 | |
| | CR BM− | 2 | 2 | |
| | CR | 66.7% | 66.7% | |
| | Sensitivity | | | |
| | CR PPV | 17.4% | 18.2% | |
| | OR BM+ | 12 | 10 | |
| | OR BM− | 5 | 7 | |
| | OR | 70.6% | 58.8% | |
| | Sensitivity | | | |
| | OR PPV | 52.2% | 45.5% | |
| Prognostic | Cox HR | 0.87 | 0.85 | Lenz et al., |
| Effects | Cox p | 0.39 | 0.33 | 2008 |
| Model | Supervision | Comp2 | CG26 | (N = 414) |
| Details | | Pseudolabels | probabilities | |
| | Form | NTP + LDA | Linear model | |
| | Genes | 26 | 49 | |
| | Shared | — | 18 | |
| | with CG26 | | | |
| | Shared | 18 | — | |
| | with M1 | | | |
| | Shared | 13 | 30 | |
| | with M2 | | | |
| | Unique genes | 1 | 13 | |

In summary, a proof of principle study was conducted to translate the classifier developed using research assays into a clinical viable diagnostic. Study objectives were to simplify the model in order to facilitate robust procedures, to place the model in the measurement scale of a clinically viable platform (e.g., Nanostring nCounter devices), and to verify that the bridged model recapitulates the information derived during the prior training exercise. The study fulfilled these objectives by defining a linear model of approximately 50 genes and corresponding threshold used to stratify samples, which resulted in statistically identical performance to the original model.

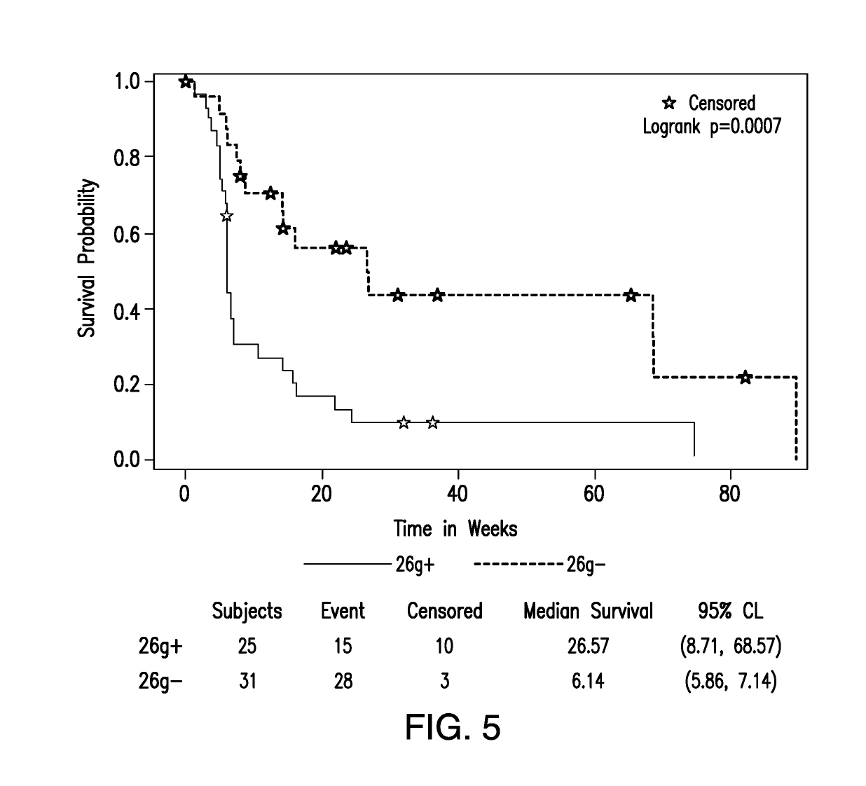

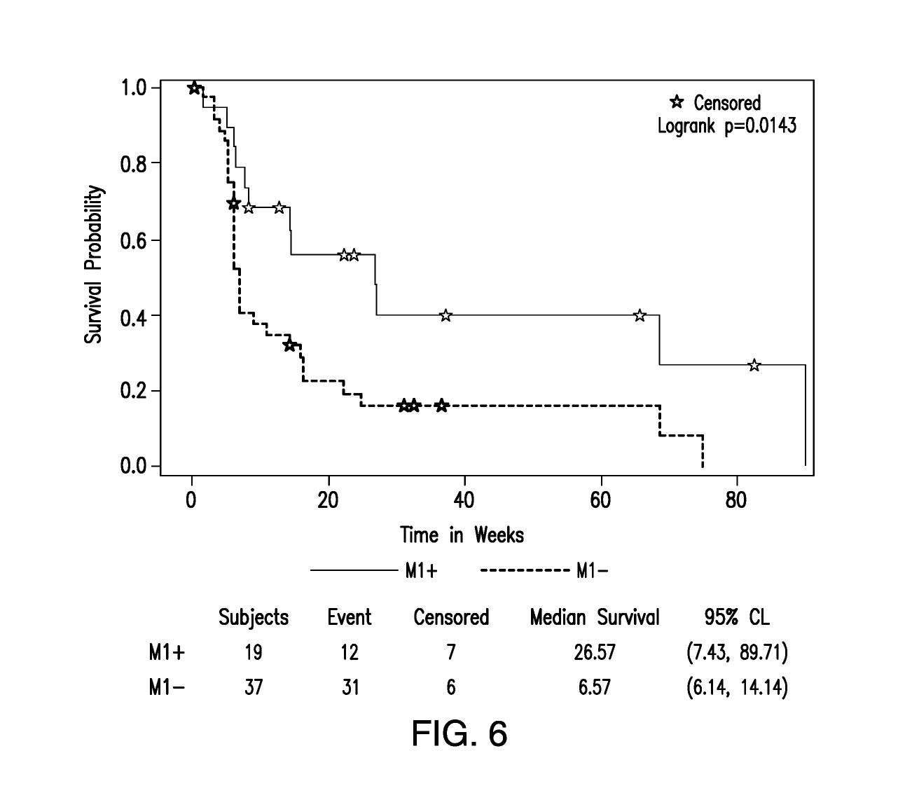

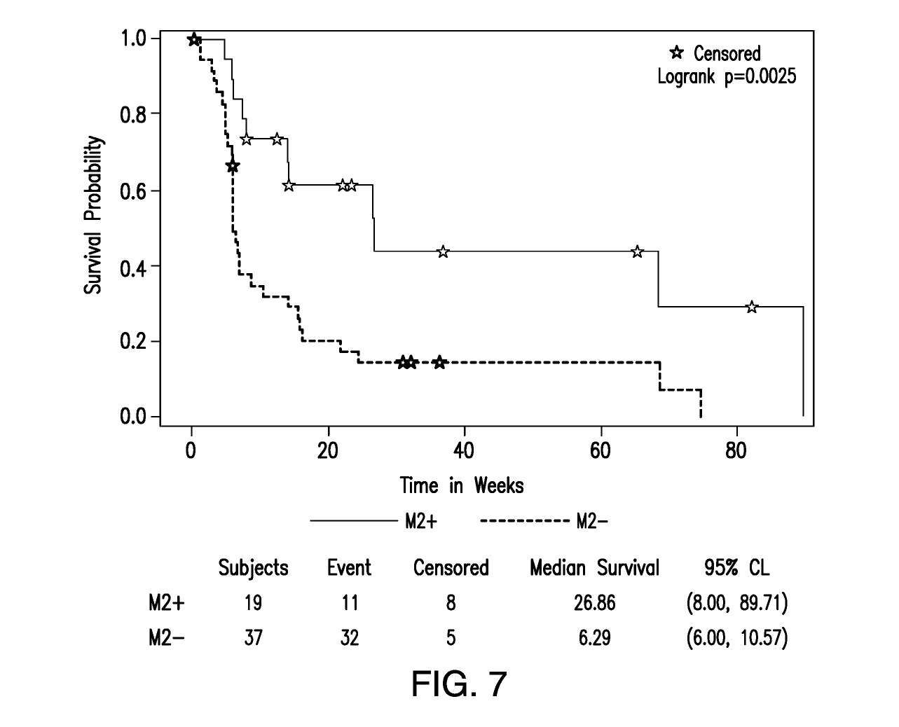

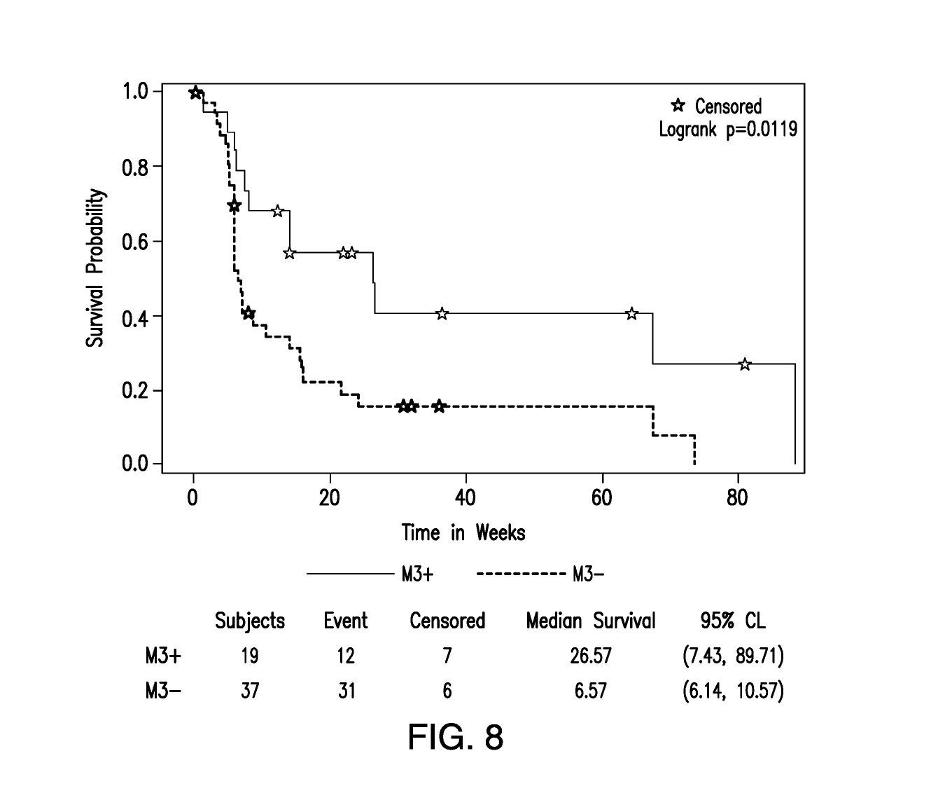

Non-limiting examples of using genes classifiers (e.g., the original 26 gene classifier or genes disclosed in Section 5.2) to predict progression free survival of NOS-DLBCL patients treated with Compound A are shown in FIGS. 5-8. FIG. 5 is Kaplan Meier plot for the 26 gene classifier in NOS-DLBCL patients treated with Compound A. FIG. 6 is Kaplan-Meier plot for the M1 gene classifier (disclosed in Table 1) in NOS-DLBCL patients treated with Compound A. FIG. 7 is Kaplan Meier plot for the M2 gene classifier (disclosed in Table 2) in NOS-DLBCL patients treated with Compound A. FIG. 8 is Kaplan Meier plot for the M3 gene classifier (disclosed in Table 3) in NOS-DLBCL patients treated with Compound A. All these Kaplan Meier plots demonstrate that these gene classifiers can be used to effectively predict DLBCL patient response to compound treatment and progression free survival.

5.10.5 Biological Interpretation of the DLBCL Subgroups

The biology of the subgroups discovered using the classifiers trained as described above was interpreted. A binary comparison between two transcriptomic phenotypes, “phenotype 1” (“B-cell”, “proliferation”, “high tumor cellularity”) and “phenotype 2” (Host interactions”, “immune response”), was made using prior biological knowledge.

5.10.5.1 Gene Expression Deconvolution Analysis

Since samples from patients may contain different cellular populations describing a complex scenario, gene expression deconvolution analysis is performed.

A possible explanation for the gene expression phenotype is that it reflects a change in the cellular composition. Thus, it was sought to determine if any changes in cellular fractions were associated with the gene expression phenotypes which produced the biomarkers (classifiers trained on Lenz cohort data). Four transcriptome based estimators of cellular phenotypes were selected for evaluation of the Lenz cohort. The estimators include GSVA (Hanzelmann et al, BMC Bioinformatics, 2013), unmix, CIBERSORT (Newman et al., Nat. Methods, 2015), and xCell (Aran et al., Genome Biol. 2017). GSVA was provided gene lists of immune cell types from Bindea et al., (2013), and unmix was provided the complete data matrix from the same work to represent pure samples of each cell type. The alpha parameter for unmix was set based on the DESeq2 dispersion estimate using a mean fit. CIBERSORT provides model based estimates from these same immune profiles, and xCell provides model based estimates from a broader panel of cell types. The array based models were used for both. The resulting estimates were tested for association with the gene expression phenotype using Spearman's rank correlation. All estimators were collected into a single matrix and evaluated by hierarchical clustering (with Pearson correlation, and average linkage) to evaluate concordance of the results.

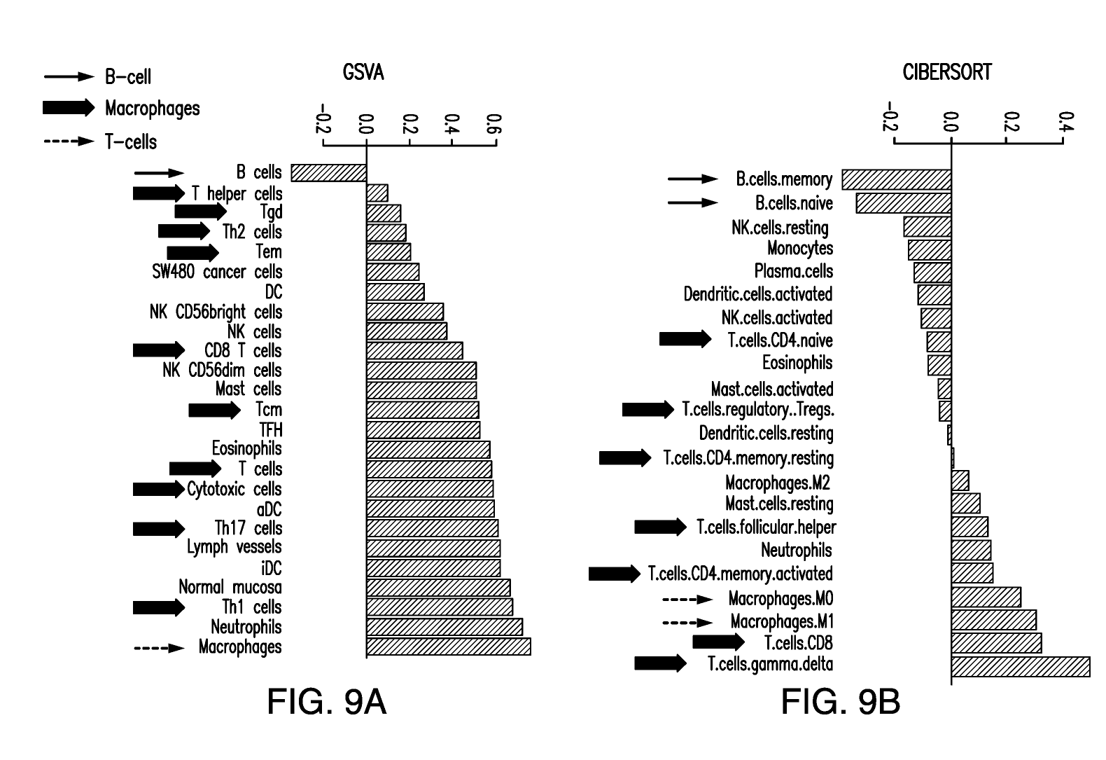

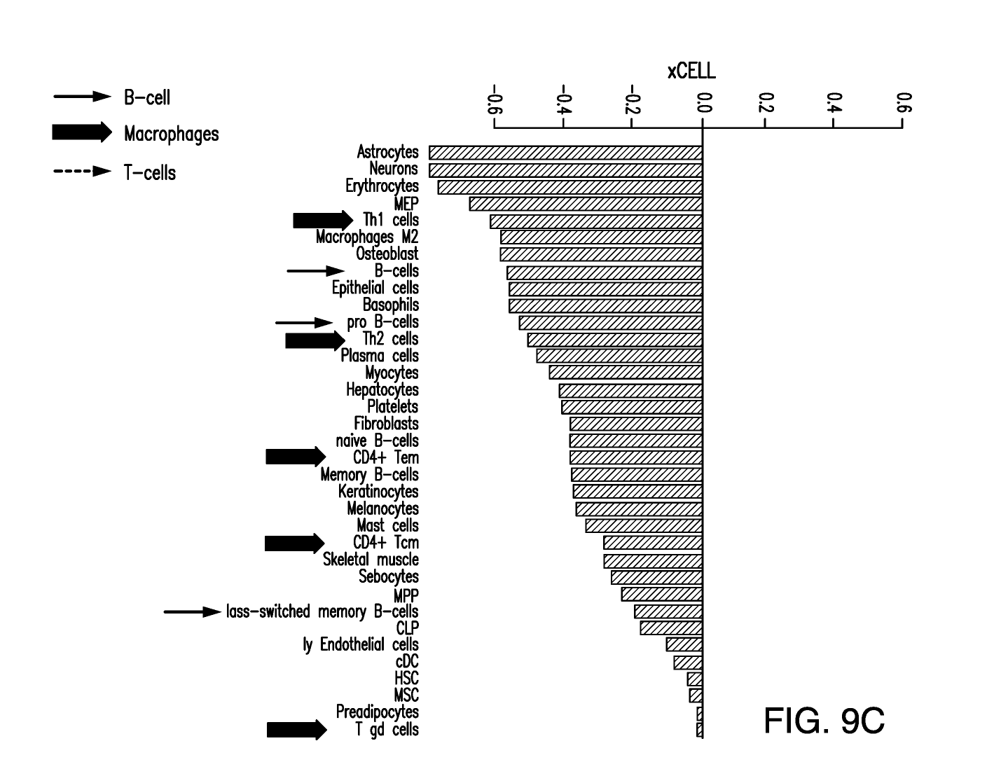

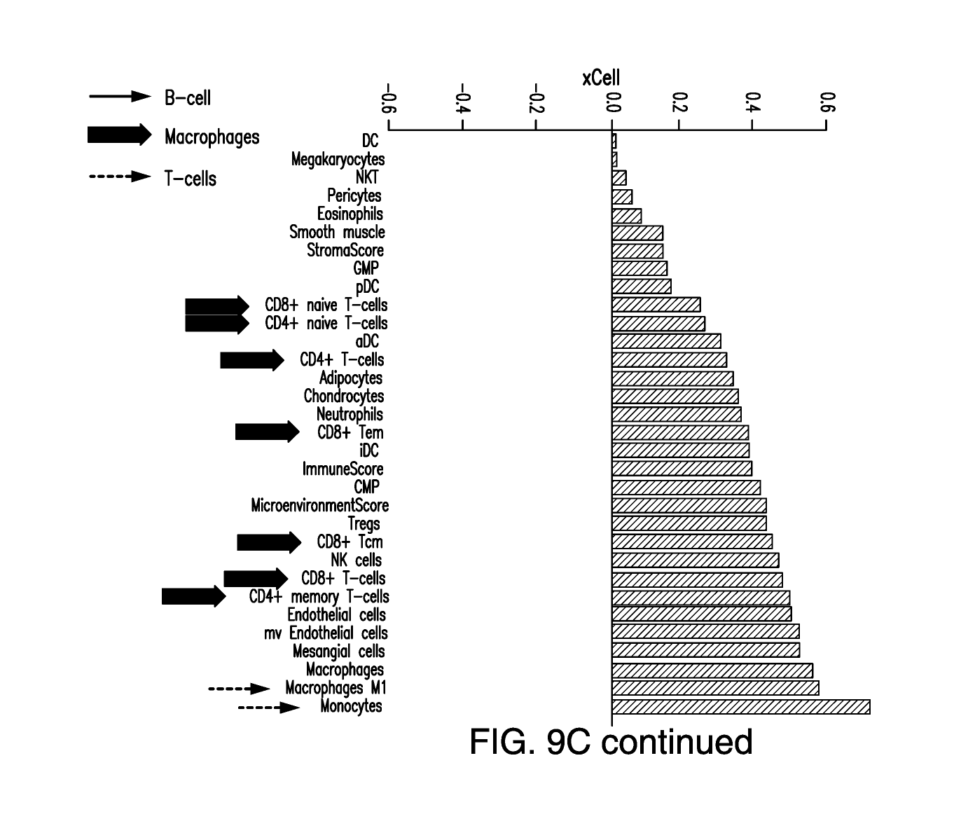

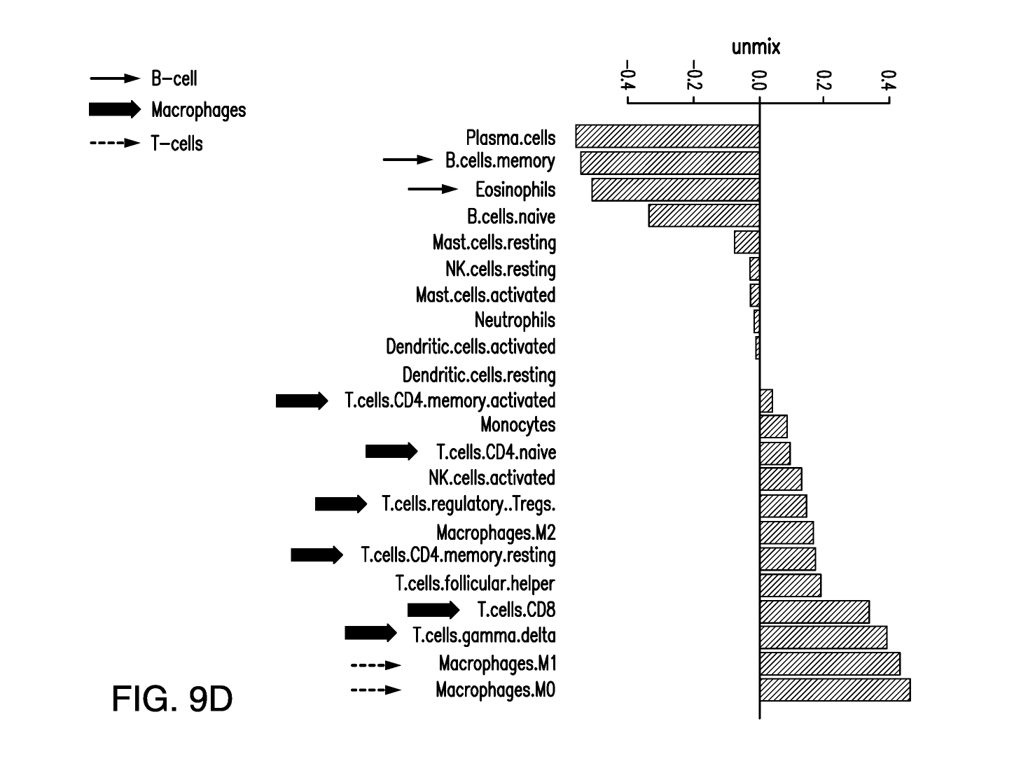

All four methods agree that B-cell phenotype is of higher magnitude in phenotype 1, and macrophage and T-cell phenotypes are of higher magnitude in phenotype 2. The estimated cellular proportions on Lenz cohort dataset using the above said methods shown in FIGS. 9A-9D (FIG. 9A: GSVA; FIG. 9B: CIBESORT; FIG. 9C: xCell; FIG. 9D: unmix).

5.10.5.2 Prediction of Phenotypes in the Compound A Cohort

The genes classifiers (e.g., the original 26 gene classifier or genes disclosed in Section 5.2) trained on the Lenz cohort was used to predict phenotype (e.g., different cellular composition) in the Compound A cohort. For example, the two subgroups of the Compound A cohort are identified as 26g+ and 26g− subgroups respectively when the original 26 gene classifier was used as the biomarker (classifier).

Fluorescent Multiplex Immunohistochemistry (IHC) Method

Two immunofluorescence Multiplex panels, PD-1 and PD-L1 6-plex IHC panels, were performed using Opal 7-Color Automation IHC Kit (PerkinElmer, Waltham, Mass.; Catalog No. NEL801001KT) on a fully automated BOND RX IHC & ISH stainer (Leica Microsystems, Buffalo Grove, Ill.). Formalin fixed paraffin embedded (FFPE) normal tonsil and DLBCL samples (Avaden Bioscience, Seattle, Wash.) were sectioned at four micron thickness and deparaffinized in Bond Dewax solution at 72° C. Antigen retrieval was performed in Epitope Retrieval 2 (ER2, pH 9.0) for 20 min at 100° C. The first round of immunostaining consisted of the following steps at room temperature: tissues were blocked with Antibody Diluent/Blocking Solution for 10 min followed by a 30 min incubation of the first primary antibody. Opal Polymer Horseradish Peroxidase (HRP) for either mouse or rabbit primaries was dispensed to the tissue sections and incubated for 10 min. Opal fluorophore solution at a 1:100 dilution was applied to the tissues and incubated for 10 min. After the first round staining, the antibody was stripped in Epitope Retrieval 1 (ER1, pH 6.0) for 20 min at 95° C. Sequentially, the second to sixth rounds of immunostaining were performed. Slides were counterstained with spectral DAPI for 5 min and rinsed in water. ProLong Diamond Antifade Mountant (Life Technology, Carlsbad, Calif., Catalog No. P36961) was then applied and slides were coverslipped.

Multiplex Image Acquisition and Analysis

We used Perkin Elmer Vectra multispectral IHC image platform to acquire PD-1 and PD-L1 multiplex images at both 4× and 20× magnification. After whole slide scan at 4× was completed, the regions of interest on the 4× images were manually outlined and the entire selected regions at 20× were automatically scanned.

A set of single fluorescence labeled DLBCL slides and a control slide without any fluorescence labeling were scanned for building referenced spectral libraries of each fluorophore using Perkin-Elmer's InForm software. The intrinsic autofluorescence spectral library was built using the image acquired from the control section. Those spectral libraries were used to convert the raw multispectral images to the component images for further image analysis.

In 20× images, InForm is used to identify individual cell nuclei and to phenotype single positive immune marker cells. To optimize the outcome of nuclear segmentation and immune cell phenotyping, about thirty representative images were sampled from all 20× images, and loaded them into InForm to determine the optimized nuclear segmentation and phenotyping settings. The preliminary segmentation and immune cell phenotyping outcomes of the representative images were evaluated and approved by pathologist before starting automatic imaging analysis onto all 20× images.

The cell coordinate and phenotype ID of all 20× images of the section were recorded in the txt files. The calculation of the immune cell density and location from the 20× images of the same section was completed using Matlab (Mathworks, Inc). To quantify the cell density and location of the dual positive immune markers, only the cells expressing both mono-positive marker were selected. For triple immune marker positive cell quantification, exact algorithms as searching dual positive immune cells were applied.

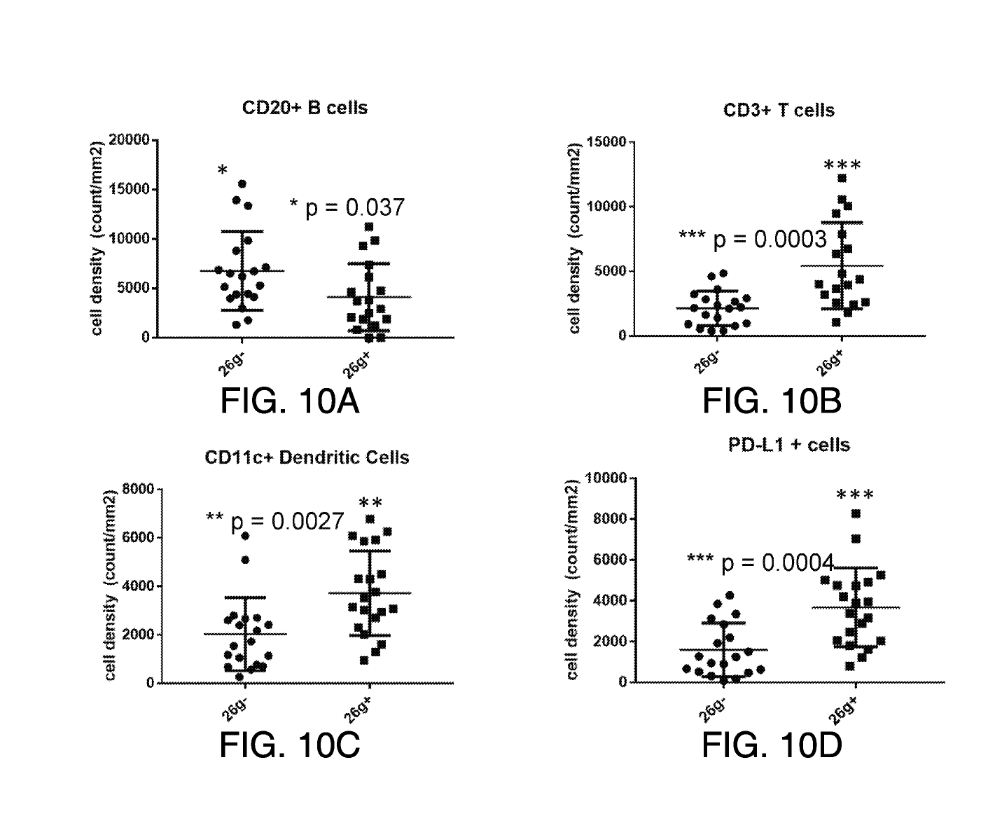

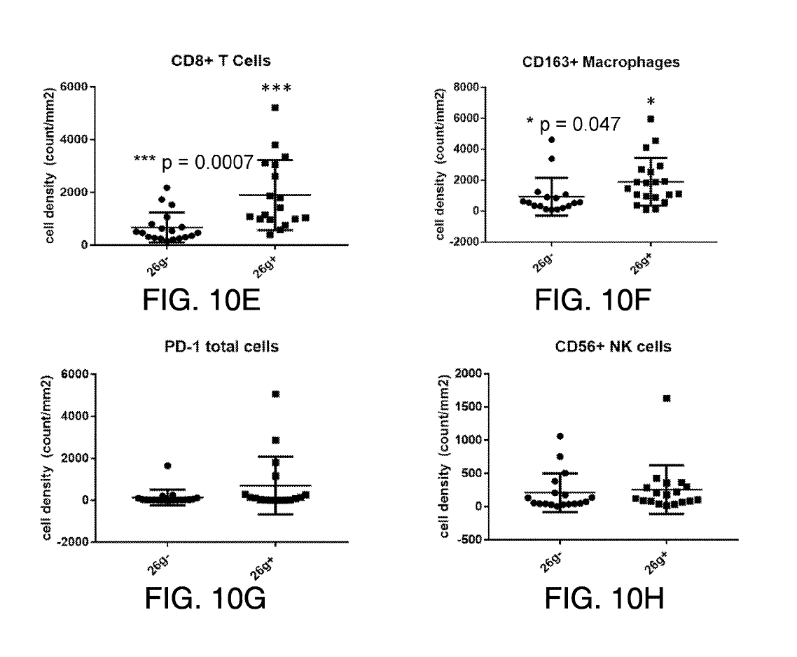

The cellular composition of different cells in the two phenotype subgroups (26g+ and 26−) are shown in FIGS. 10A-H. FIG. 10A displays higher proportion of CD20+ B cells in the 26g− phenotype subgroup than in the 26g+ phenotype subgroup. FIGS. 10B and 10E display lower proportion of CD3+ and CD8+ T cells in the 26− phenotype subgroup than in the 26+ phenotype subgroup. FIG. 10F displays lower proportion of CD163+ macrophage cells in the 26g− phenotype subgroup than in the 26g+ phenotype subgroup. FIG. 10C displays lower proportion of CD11c+ dendritic cells in the 26g− phenotype subgroup than in the 26g+ phenotype subgroup. FIG. 10D displays lower proportion of PD-L1+ cells in the 26g− phenotype subgroup than in the 26g+ phenotype subgroup.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1.-51. (canceled)

52. A method for predicting the responsiveness of a patient having a hematological cancer to a treatment compound, comprising:
(a) obtaining a biological sample from the patient having the hematological cancer;
(b) determining the expression level of one, two, three, four, five, or more of the genes selected from the groups consisting of:
(i) BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, ZNF215,
(ii) ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, ZNF215,
(iii) C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, ZNF215, and/or
(iv) C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, ZNF215; and
(c) comparing the expression level of the one, two, three, four, five, or more of the genes selected from the groups consisting of:
(i) BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, ZNF215,
(ii) ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, ZNF215,
(iii) C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, ZNF215, and/or
(iv) C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, ZNF215
in the biological sample with a reference expression level of the same genes;
wherein the differential expression level of the one, two, three, four, five, or more of the genes in the biological sample relative to the reference expression level of the one, two, three, four, five, or more of the genes indicates that the first patient will be responsive to the treatment compound; and
wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

53. The method of claim 52, wherein the reference sample is synthetically derived.

54. The method of claim 52, wherein the biological samples are tumor biopsy samples.

55. The method of claim 52, wherein the determining step comprises detecting the presence and/or amount of a complex in the biological sample, wherein the presence and/or amount of the complex indicates the expression level of the genes.

56. The method of claim 55, wherein the complex is a hybridization complex.

57. The method of claim 56, wherein the hybridization complex is attached to a solid support.

58. The method of claim 57, wherein the hybridization complex is detectably labeled.

59. The method of claim 52, wherein the hematological cancer is selected from the group consisting of leukemia, lymphoma, and multiple myeloma.

60. The method of claim 59, wherein the lymphoma is DLBCL.

61. The method of claim 60, wherein the DLBCL is refractory in the first patient.

62. The method of claim 60, wherein the DLBCL is relapsed in the first patient.

63. The method of claim 60, wherein the DLBCL is a germinal center B-cell-like subtype (GCB-DLBCL) in the first patient.

64. The method of claim 60, wherein the DLBCL is activated B-cell-like subtype (ABC-DLBCL).

65. The method of claim 60, wherein the DLBCL is not otherwise classified (NOS-DLBCL).

66. The method of claim 52, wherein the expression level of one, two, three, four, five, or more of the genes selected from the group consisting of BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, and ZNF215 are determined and compared.

67. The method of claim 52, wherein the expression level of one, two, three, four, five, or more of the genes selected from the group consisting of ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, and ZNF215 are determined and compared.

68. The method of claim 52, wherein the expression level of one, two, three, four, five, or more of the genes selected from the group consisting of C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, and ZNF215 are determined and compared.

69. The method of claim 52, wherein the expression level of one, two, three, four, five, or more of the genes selected from the group consisting of C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, and ZNF215 are determined and compared.

70. A method for predicting the responsiveness of a patient having a hematological cancer to a treatment compound, comprising:
(a) obtaining a first tumor sample from a first patient having the hematological cancer,
(b) measuring the proportion of B cells and/or T cells in the first tumor sample from a first patient, and
(c) comparing the proportion of B cells and/or T cells in the first tumor sample from a first patient with the proportion of B cells or T cells in a second tumor sample from a second patient having the same type of hematological cancer, wherein the second patient's hematological cancer is clinically insensitive to treatment with an immunomodulatory therapy, and
wherein a different proportion of B cells and/or T cells in the first tumor sample is measured relative the proportion of B cells and/or T cells in the second tumor sample indicates that the hematological cancer in the first patient will be clinically sensitive to treatment with the immunomodulatory therapy,
wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.

71. A kit for predicting the responsiveness of a patient having a hematological cancer to a treatment compound, comprising an agent for determining the expression levels of the genes selected from the groups consisting of:
(i) BICC1, C10orf54, C1RL, C20orf112, C8orf4, CCDC88C, CD97, CEBPB, CFB, CHRDL1, CIRH1A, CPSF3, CSF1R, DDR2, DLGAP5, DUSP1, EMR2, EPB41L3, FBXO32, GAS2L1, GINS4, IFITM2, KCNMB1, LGALS3BP, LRP11, MEGF6, MEIS1, MYO1F, PDGFC, PRNP, PTPRM, RAB31, RBBP8, RUVBL1, SEMA3C, SERPING1, SMOC2, SNRPD1, TBC1D2B, THEMIS2, TMEM173, TPSAB1, TRIP13, TXNIP, ULK1, WDR12, WWTR1, XAF1, ZNF215,
(ii) ALKBH2, AOAH, C10orf54, C1RL, C8orf4, CD84, CD97, CDC20, CEBPB, CEBPD, CHRDL1, CIRH1A, CPSF3, CYP1B1, FBXO32, FOSL2, GAS2L1, GBP3, GINS4, GNAQ, GPR155, GPRIN3, HJURP, KCNMB1, LRP11, MAFB, MEGF6, MEIS1, MYO1F, MYOF, PCSK5, PDGFC, PHACTR2, PTPRM, RAB31, RALGDS, RUVBL1, SASH1, SERPING1, SMOC2, SNRPD1, SSH1, TBC1D2B, TMEM173, TP53INP2, TRIP13, ULK1, WWTR1, XAF1, ZNF215,
(iii) C10orf54, C1RL, C20orf112, C8orf4, CCDC88C, CILP, CIRH1A, CLU, CPVL, CSF1R, CTSB, EPB41L3, FBXO32, IFI44, LRP11, MEGF6, MEIS1, PLAT, SERPING1, SPC25, THEMIS2, TPSAB1, ULK1, XAF1, ZNF215, and/or
(iv) C10orf54, C1RL, C20orf112, C8orf4, CCNB1, CDC6, CILP, CLU, CPSF3, CPVL, CSF1R, CTSB, EPB41L3, FBXO32, IFI44, MEIS1, PHACTR2, PLAT, RUVBL1, SERPING1, SNRPD1, TRIP13, XAF1, ZNF215,
or a subset thereof in a biological sample obtained from the patient prior to the compound treatment, wherein the treatment compound is 3-(4-amino-1-oxo-1,3-dihydro-isoindol-2-yl)-piperidine-2,6-dione (lenalidomide), 3-(5-amino-2-methyl-4-oxo-4H-quinazolin-3-yl)-piperidine-2,6-dione (Compound A), or a stereoisomer, pharmaceutically acceptable salt, solvate, hydrate, co-crystal, clathrate, or a polymorph thereof.
